(19) United States
(12) Patent Application Publication
(10) Pub. No.: US 2022/0251085 A1
(43) Pub. Date: Aug. 11, 2022

(54) CYSTEINE BINDING COMPOSITIONS AND METHODS OF USE THEREOF

(71) Applicant: University of Virginia Patent Foundation, Charlottesville, VA (US)

(72) Inventors: Ku-Lung Hsu, Charlottesville, VA (US); Adam Libby, Charlottesville, VA (US); Andy Heindel, Charlottesville, VA (US); Jeffrey W. Brulet, Glen Allen, VA (US); Boobalan Pachaiyappan, Charlottesville, VA (US)

(73) Assignee: University of Virginia Patent Foundation, Charlottesville, VA (US)

(21) Appl. No.: 17/629,303

(22) PCT Filed: Jul. 21, 2020
(86) PCT No.: PCT/US2020/042915

§ 371 Date: Jan. 21, 2022

Related U.S. Application Data
(60) Provisional application No. 62/876,703, filed on Jul. 21, 2019.

(51) Int. Cl. C07D 473/00; G01N 33/58; G01N 33/68; C07K 2/00

(52) U.S. Cl. CPC C07D 473/00; G01N 33/582; C07K 2/00; G01N 33/6851; G01N 33/6815

(57) ABSTRACT

Purine-derived covalent probes (e.g., halo or di-halo-substituted purine based covalent probes) and related ligands are described. The compounds can be used to identify reactive nucleophilic amino acid residues, such as reactive cysteine residues, in proteins and to modify the activity of proteins with reactive nucleophilic amino acid residues (e.g., reactive cysteine residues) via the formation of protein adducts comprising the ligands Modified proteins prepared from the probes and ligands are also described.

### CROSS-REFERENCE TO RELATED APPLICATION

This application claims the benefit of U.S. Provisional Patent Application Ser. No. 62/876,703, filed Jul. 21, 2019, the disclosure of which is incorporated herein by reference in its entirety.

### GRANT STATEMENT

This invention was made with government support under Grant No. GM 007055 awarded by National Institutes of Health. The Government has certain rights in the invention.

### TECHNICAL FIELD

The presently disclosed subject matter relates to diagnostics and therapeutics. In particular, it relates to tunable chemistry for global discovery of protein function and ligands, particularly with respect to design of purine-based probes for use in protein function analyses and the identification of related ligands that can interact with reactive amino acid residues in proteins.

### BACKGROUND

Chemical proteomics is a powerful technology for ascribing function to the vast number of uncharacterized proteins in the human proteome^{1,2}. This proteomic method employs probes designed with reactive groups that exploit accessibility and reactivity of binding sites to covalently label active proteins with reporter tags for function assignment and inhibitor development³. Selective probes resulting from competitive screening efforts serve as enabling, and often first-in-class, tools for uncovering biochemical and cellular functions of proteins (e.g. serine hydrolases⁴, proteases⁵, kinases⁶, phosphatases⁷, and glycosidases⁸) and their roles in contributing to human physiology and disease. The basic and translational opportunities afforded by chemical proteomics has prompted exploration of new biocompatible chemistries for broader exploration of the proteome.

Covalent probes used for chemical proteomics range from highly chemoselective fluorophosphonates for catalytic serines⁹ to general thiol alkylating agents and amine-reactive esters of cysteines¹⁰ and lysines¹¹, respectively. The ability to globally measure protein functional states and selectively perturb proteins of interest has substantially augmented the basic understanding of protein function in cell and animal models^{1,3}. Exploration of new redox-based oxaziridine chemistry, for example, identified a conserved hyper-reactive methionine residue (M169) in redox regulation of mammalian enolase¹². Hydrazine probes revealed a novel N-terminal glyoxylyl post-translational modification on the poorly characterized protein SCRN3¹³. More recent exploration of photoaffinity probes has facilitated global evaluation of reversible small molecule-protein interactions to expand the scope of proteins available for chemical proteomic profiling¹⁴.

However, there remains an ongoing need for additional covalent probes for chemical proteomic profiling, particularly those that provide a scaffold amenable to optimization and drug development.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method for identifying a reactive amino acid residue of a protein, the method comprising: (a) providing a protein sample comprising isolated proteins, living cells, a cell lysate, or a biological organism; (b) contacting the protein sample with a probe compound of Formula (I) for a period of time sufficient for the probe compound to react with at least one reactive amino acid in a protein in the protein sample, thereby forming at least one modified amino acid residue; and (c) analyzing proteins in the protein sample or removed from the protein sample to identify at least one modified amino acid residue, thereby identifying at least one reactive amino acid residue of a protein; wherein the probe compound has a structure of Formula (I):

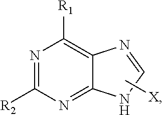

wherein: X is a monovalent moiety comprising an alkyne moiety, a fluorophore moiety, a detectable labeling group, or a combination thereof; and R₁ and R₂ are independently selected from the group comprising H, halo, amino, alkyl, alkoxy, alkylthio, alkylamino, aryloxy, arylthiol, and arylamino, subject to the proviso that at least one of R₁ and R₂ is halo.

In some embodiments, the probe compound of Formula (I) has a structure of Formula (Ia):

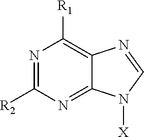

or a structure of Formula (Ib):

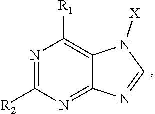

wherein: X is a monovalent moiety comprising an alkyne moiety, a fluorophore moiety, a detectable labeling group, or a combination thereof; and R₁ and R₂ are independently selected from the group comprising H, halo, amino, alkyl, alkoxy, alkylthio, aryloxy, arylthiol, and arylamino, subject to the proviso that at least one of R₁ and R₂ is halo.

In some embodiments, the reactive amino acid residue is a cysteine residue. In some embodiments, the modified amino acid residue has a structure of Formula (IIa-i):

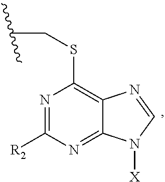

a structure of Formula (IIb-i):

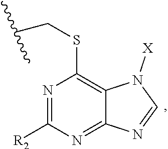

a structure of Formula (IIa-ii):

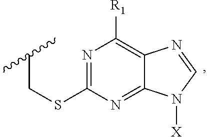

or a structure of Formula (IIb-ii):

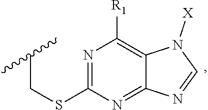

wherein: X is a monovalent moiety comprising an alkyne moiety, a fluorophore moiety, a detectable labeling group, or a combination thereof; R₁ is selected from the group comprising H, halo, amino, alkyl, alkoxy, alkylthio, alkylamino, aryloxy, arylthio, and arylamino; and R₂ is selected from the group comprising H, halo, amino, alkyl, alkoxy, alkylthio, alkylamino, aryloxy, arylthio, and arylamino.

In some embodiments, R₁ and R₂ are selected from H, halo, and amino or R₁ and R₂ are selected from H and halo. In some embodiments, R₁ is chloro or fluoro. In some embodiments, R₂ is chloro or fluoro. In some embodiments, X is —CH₂—C≡CH.

In some embodiments, the probe compound is selected from the group comprising 2,6-dichloro-7-(prop-2-yn-1-yl)-7H-purine, 2,6-dichloro-9-(prop-2-yn-1-yl)-9H-purine, 6-chloro-7-(prop-2-yn-1-yl)-7H-purine, 6-chloro-9-(prop-2-yn-1-yl)-9H-purine, 2-chloro-7-(prop-2-yn-1-yl)-7H-purine, 2-chloro-9-(prop-2-yn-1-yl)-9H-purine, 2,6-difluoro-7-(prop-2-yn-1-yl)-7H-purine, 2,6, -difluoro-9-(prop-2-yn-1-yl)-9H-purine, 6-chloro-2-fluoro-7-(prop-2-yn-1-yl)-7H-purine, 6-chloro-2-fluoro-9-(prop-2-yn-1-yl)-9H-purine, 6-chloro-2-amino-7-(prop-2-yn-1-yl)-7H-purine, and 6-chloro-2-amino-9-(prop-2-yn-1-yl)-9H-purine.

In some embodiments, the probe compound has a structure of Formula (Ib). In some embodiments, the probe compound is 2,6-dichloro-7-(prop-2-yn-1-yl)-7H-purine.

In some embodiments, the analyzing of step (c) further comprises tagging the at least one modified reactive amino acid residue with a compound comprising a detectable labeling group, thereby forming at least one tagged reactive amino acid residue comprising said detectable labeling group. In some embodiments, the detectable labeling group comprises biotin or a biotin derivative, optionally wherein the biotin derivative is desthiobiotin.

In some embodiments, the tagging comprises reacting an alkyne group in the X moiety of the at least one modified reactive amino acid residue with a compound comprising (i) an azide moiety and (ii) the detectable labeling group, optionally via a copper-catalyzed azide-alkyne cycloaddition (CuAAC) coupling reaction. In some embodiments, the analyzing further comprises digesting proteins with trypsin to provide a digested protein sample comprising a protein fragment comprising the at least one tagged reactive amino acid moiety comprising the detectable group. In some embodiments, the analyzing further comprises enriching the digested protein sample for the detectable labeling group, optionally wherein the enriching comprises contacting the digested protein sample with a solid support comprising a binding partner of the detectable labeling group. In some embodiments, the analyzing further comprises analyzing the enriched digested protein sample via liquid chromatography-mass spectrometry (LC-MS).

In some embodiments, the protein sample is a biological organism, optionally a mammal; wherein contacting the protein sample with the probe compound of Formula (I) comprises administering the probe compound of Formula (I) to the biological organism, optionally via oral administration or injection; and wherein prior to analyzing the proteins, tissues are removed from the biological organism and homogenized.

In some embodiments, providing the protein sample further comprises separating the protein sample into a first protein sample and a second protein sample; contacting the protein sample with a probe compound of Formula (I) comprises contacting the first protein sample with a first probe compound of Formula (I) at a first probe concentration for a first period of time and contacting the second protein sample with one of the group consisting of: (b1) a second probe compound of Formula (I) at the first probe concentration for the first period of time, (b2) the first probe compound of Formula (I) at a second probe concentration for the first period of time, and (b3) the first probe compound of Formula (I) at the first probe concentration for a second period of time; thereby forming at least one modified reactive amino acid residue in said first and/or said second protein sample; and analyzing proteins comprises analyzing the first and second protein samples to determine the presence and/or identity of a modified reactive amino acid residue in the first sample and the presence and/or identity of a modified reactive amino acid residue in the second sample.

In some embodiments, the protein sample comprises living cells and wherein providing the protein sample further comprises separating the protein sample into a first protein sample and a second protein sample and culturing the first protein sample in a first cell culture medium comprising heavy isotopes prior to the contacting of step (b), optionally wherein the first cell culture medium comprises ¹³C- and/or ¹⁵N-labeled amino acids, further optionally wherein the first cell culture medium comprises ¹³C-¹⁵N-labeled lysine and arginine; and culturing the second protein sample in a second cell culture medium, wherein said second cell culture medium comprises a naturally occurring isotope distribution, prior to the contacting of step (b). In some embodiments, one of the first and the second protein sample is cultured in the presence of an inhibitor of an enzyme known or suspected of being present in said first or second protein sample.

In some embodiments, the probe compound of Formula (I) comprises a detectable labeling group comprising a heavy isotope or wherein the analyzing of step (c) further comprises tagging the at least one modified amino acid residue with a compound comprising a detectable labeling group comprising a heavy isotope, optionally wherein the heavy isotope is carbon-13.

In some embodiments, the presently disclosed subject matter provides a probe compound for detecting a reactive amino acid residue, optionally a reactive cysteine residue, in a protein, wherein the probe compound is selected from the group comprising 2,6-difluoro-7-(prop-2-yn-1-yl)-7H-purine, 2,6-difluoro-9-(prop-2-yn-1-yl)-9H-purine, and 6-chloro-2-fluoro-7-(prop-2-yn-1-yl)-7H-purine.

In some embodiments, the presently disclosed subject matter provides a compound having the structure of Formula (III):

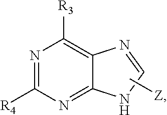

wherein: Z is selected from the group comprising cycloalkyl, acyl, substituted acyl, —S(═O)₂—R₅, —S(═O)₂—N(R₆)₂, —S(═O)₂—O—R₇, and

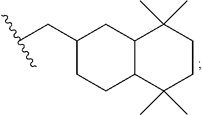 [SMILES: *CC1CCC2C(C1)C(C)(C)CCC2(C)C]

R₃ and R₄ are independently selected from H, halo, alkyl, alkoxy, alkylamino, alkylthio, aryloxy, arylamino, and arylthiol, subject to the proviso that at least one of R₃ and R₄ is halo, optionally chloro or fluoro; R₅ is heterocyclyl, substituted heterocyclyl, aryl or substituted aryl; each R₆ is selected from H, alkyl, substituted alkyl, aralkyl, substituted aralkyl, aryl, and substituted aryl, or wherein the two R₆ together form an alkylene group; and R₇ is selected from alkyl, substituted alkyl, aralkyl, substituted aralkyl, aryl and substituted aryl.

In some embodiments, the compound of Formula (III) has a structure of Formula (IIIa):

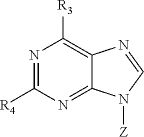

or a structure of Formula (IIIb):

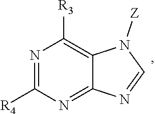

wherein: Z is selected from the group comprising cycloalkyl, acyl, substituted acyl, —S(═O)₂—R₅, —S(—O)₂—N(R₆)₂, —S(═O)₂—O—R₇, and

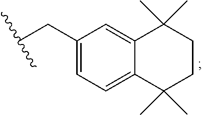 [SMILES: *Cc1ccc2c(c1)C(C)(C)CCC2(C)C]

R₃ and R₄ are independently selected from H, halo, alkyl, alkoxy, alkylamino, alkylthio, aryloxy, arylamino, and arylthiol, subject to the proviso that at least one of R₃ and R₄ is halo, optionally chloro or fluoro; R₅ is heterocyclyl, substituted heterocyclyl, aryl or substituted aryl; each R₆ is selected from H, alkyl, substituted alkyl, aralkyl, substituted aralkyl, aryl, and substituted aryl, or wherein the two R₆ together form an alkylene group; and R₇ is selected from alkyl, substituted alkyl, aralkyl, substituted aralkyl, aryl and substituted aryl.

In some embodiments, R₃ is selected from chloro, methyl, —SH—(CH₂)₃CH₃; —NH(CH₂)₃CH₃; and —O—(C₆H₄)CH₃. In some embodiments, R₄ is chloro or fluoro. In some embodiment, Z is acetyl, n-hexanoyl, n-dodecanoyl; cyclohexyl, —S(═O)₂—R₅, —S(═O)₂—N(R₆)₂, —S(═O)₂—O—R₇, and

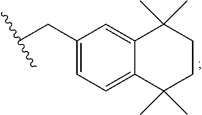

wherein R₅ is heterocyclyl or substituted phenyl; optionally wherein the substituted phenyl is alkoxy- or halo-substituted phenyl; each R₆ is selected from alkyl and aralkyl, optionally methyl, ethyl or benzyl; and R₇ is alkyl, optionally methyl. In some embodiments, Z is selected from

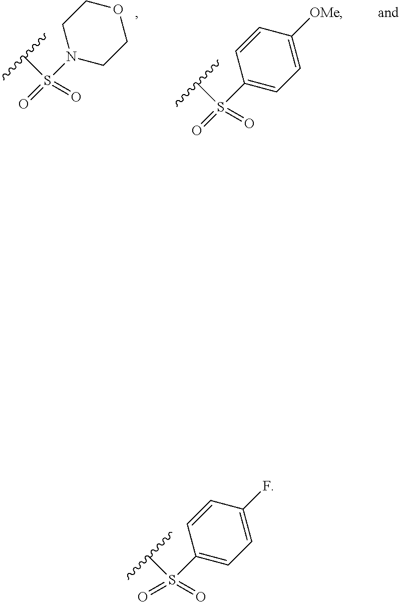 [SMILES: *S(=O)(=O)N1CCOCC1 ; *S(=O)(=O)c1ccc(OC)cc1 ; *S(=O)(=O)c1ccc(F)cc1]

In some embodiments, the compound is selected from the group comprising 4-((2,6-dichloro-7H-purin-7-yl)sulfonyl)morpholine, 4-((2,6-dichloro-9H-purin-9-yl)sulfonyl)morpholine, 2,6-dichloro-7-((4-fluorophenyl)sulfonyl)-7H-purine, 2,6-dichloro-9-((fluorophenyl)sulfonyl)-9H-purine, 2,6-dichloro-7-((4-methoxyphenyl)sulfonyl)-7H-purine, 2,6-dichloro-9-((4-methoxyphenyl)sulfonyl)-9H-purine, 2,6-dichloro-7-((5,5,8,8-tetramethyl-5,6,7,8-tetrahydronaphthalen-2-yl)methyl)-7H-purine, 2,6-dichloro-9-((5,5,8,8-tetramethyl-5,6,7,8-tetrahydronaphthalen-2-yl)methyl)-9H-purine, 1-(2,6-dichloro-9H-purin-9-yl)dodecan-1-one, 1-(2,6-dichloro-7H-purin-7-yl)hexan-1-one, 1-(2,6-dichloro-9H-purin-9-yl)hexan-1-one, 1-(6-chloro-2-fluoro-7H-purin-7-yl)hexan-1-one, 1-(6-chloro-2-fluoro-9H-purin-9-yl)hexan-1-one, 1-(2-chloro-6-methyl-9H-purin-9-yl)hexan-1-one, 2-chloro-9-cyclohexyl-6-methyl-9H-purine, 9-cyclohexyl-2-fluoro-6-methyl-9H-purine, 1-(6-(butylthio)-2-fluoro-9H-purin-9-yl)ethan-1-one, 1-(6-(butylthio)-2-chloro-9H-purin-9-yl)ethan-1-one, 1-(6-(butylthio)-2-chloro-7H-purin-7-yl)ethan-1-one, 1-(6-(butylthio)-2-fluoro-7H-purin-7-yl)ethan-1-one, 1-(2-chloro-6-(p-tolyloxy)-9H-purin-9-yl)ethan-1-one, 1-(2-fluoro-6-(p-tolyloxy)-9H-purin-9-yl)ethan-1-one, 1-(2-chloro-6-(p-tolyloxy)-7H-purin-7-yl)ethan-1-one, 1-(2-fluoro-6-(p-tolyloxy)-7H-purin-7-yl)ethan-1-one, 1-(6-(butylamino)-2-chloro-7H-purin-7-yl)ethan-1-one, 1-(6-(butylamino)-2-fluoro-7H-purin-7-yl)ethan-1-one, 1-(6-(butylamino)-2-chloro-9H-purin-9-yl)ethan-1-one, 1-(6-(butylamino)-2-fluoro-9H-purin-9-yl)ethan-1-one, 2,6-dichloro-N,N-diethyl-7H-purine-7-sulfonamide, 2,6-dichloro-N,N-diethyl-9H-purine-9-sulfonamide, N-benzyl-2,6-dichloro-N-methyl-9H-purine-9-sulfonamide, N-benzyl-2,6-dichloro-N-methyl-7H-purine-7H-sulfonamide, benzyl 2,6-dichloro-7H-purine-7-sulfonate, benzyl 2,6-dichloro-9H-purine-9-sulfonate, methyl 2,6-dichloro-9H-purine-9-sulfonate, and methyl 2,6-dichloro-7H-purine-7-sulfonate.

In some embodiments, the presently disclosed subject matter provides a compound where the compound is 2,6-dichloro-7-(4-nitrobenzyl)-7H-purine.

In some embodiments, the presently disclosed subject matter provides a modified cysteine-containing protein comprising a modified cysteine residue wherein the modified cysteine residue is formed by the reaction of a cysteine residue with a non-naturally occurring purine-based compound wherein said non-naturally occurring purine-based compound is a compound having a structure of Formula (I):

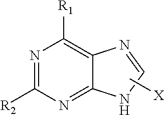

or a compound having a structure of Formula (III′):

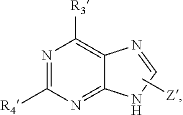

wherein: X is a monovalent moiety comprising an alkyne moiety, a fluorophore moiety, a detectable labeling group, or a combination thereof; Z′ is selected from the group comprising alkyl, optionally —CH₂—CH═CH₂, substituted alkyl, cycloalkyl, heterocycloalkyl, acyl, substituted acyl, aralkyl, substituted aralkyl, —S(═O)₂—R₅′, —S(═O)₂—N(R₆)₂, and —S(═O)₂—O—R₇; R₁ and R₂ are independently selected from the group comprising H, halo, hydroxyl, thiol, amino, alkyl, alkoxy, alkylamino, alkylthio, aryloxy, arylamino, and arylthio, subject to the proviso that at least one of R₁ and R₂ is halo; R₃′ and R₄′ are independently selected from H, halo, alkyl, alkylamino, alkylthio, alkoxy, aryloxy, arylamino, and arylthiol, subject to the proviso that at least one of R₃′ and R₄′ is halo; R₅′ is heterocyclyl, substituted heterocyclyl, aryl or substituted aryl; each R₆ is selected from H, alkyl, substituted alkyl, aralkyl, substituted aralkyl, aryl, and substituted aryl, or wherein the two R₆ together form an alkylene group; and R₇ is selected from alkyl, substituted alkyl, aralkyl, substituted aralkyl, aryl and substituted aryl.

In some embodiments, the modified cysteine-containing protein comprises at least one modified cysteine residue comprising a structure of Formula (II-i):

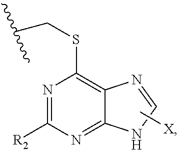

a structure of Formula (II-ii):

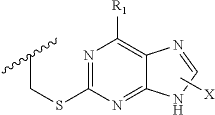

a structure of Formula (IV′-i):

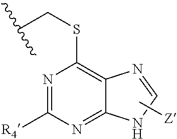

or a structure of Formula (IV′-ii):

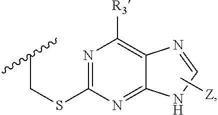

wherein: X is a monovalent moiety comprising an alkyne moiety, a fluorophore moiety, a detectable labeling group, or a combination thereof; Z′ is selected from the group comprising alkyl, optionally —CH₂—CH═CH₂, substituted alkyl, cycloalkyl, heterocycloalkyl, acyl, substituted acyl, aralkyl, substituted aralkyl, —S(═O)₂—R₅′, —S(═O)₂—N(R₆)₂, and —S(═O)₂—O—R₇; R₁ is selected from the group comprising H, halo, hydroxyl, thiol, amino, alkyl, alkoxy, alkylamino, alkylthio, aryloxy, arylamino, and arylthio; R₂ is selected from the group comprising H, halo, hydroxyl, thiol, amino, alkyl, alkoxy, alkylamino, alkylthio, aryloxy, arylamino, and arylthio; R₃′ is selected from the group comprising H, halo, alkyl, alkylamino, alkylthio, alkoxy, aryloxy, arylamino, and arylthiol; R₄′ is selected from the group comprising H, halo, alkyl, alkylamino, alkylthio, alkoxy, aryloxy, arylamino, and arylthiol; R₅′ is heterocyclyl, substituted heterocyclyl, aryl or substituted aryl; each R₆ is selected from H, alkyl, substituted alkyl, aralkyl, substituted aralkyl, aryl, and substituted aryl, or wherein the two R₆ together form an alkylene group; and R₇ is selected from alkyl, substituted alkyl, aralkyl, substituted aralkyl, aryl and substituted aryl.

In some embodiments, the modified cysteine-containing protein is selenocysteine elongation factor (eEF-Sec) modified at cysteine 442, macrophage migration inhibitory factor modified at cysteine 81; or serine/threonine protein kinase 38-like modified at cysteine 235.

In some embodiments, the presently disclosed subject matter provides a method for modulating an activity of a protein comprising a reactive cysteine residue, wherein the method comprising contacting a protein comprising a reactive cysteine residue with a compound having a structure of Formula (III′):

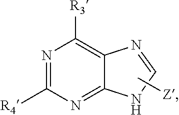

wherein: Z′ is selected from the group comprising alkyl, optionally —CH₂—CH═CH₂, substituted alkyl, cycloalkyl, heterocycloalkyl, acyl, substituted acyl, aralkyl, substituted aralkyl, —S(═O)₂—R₅′, —S(═O)₂—N(R₆)₂, and —S(═O)₂—O—R₇; R₃′ and R₄′ are independently selected from H, halo, alkyl, alkylamino, alkylthio, alkoxy, aryloxy, arylamino, and arylthio, subject to the proviso that at least one of R₃′ and R₄′ is halo; R₅′ is heterocyclyl, substituted heterocyclyl, aryl or substituted aryl; each R₆ is selected from H, alkyl, substituted alkyl, aralkyl, substituted aralkyl, aryl, and substituted aryl, or wherein the two R₆ together form an alkylene group; and R₇ is selected from alkyl, substituted alkyl, aralkyl, substituted aralkyl, aryl and substituted aryl. In some embodiments, the compound having a structure of Formula (III′) is a compound having a structure of Formula (IIIa′):

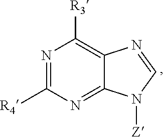

or a structure of Formula (IIIb′):

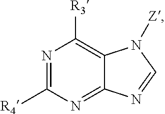

wherein Z′, R₃′, and R₄′ are as defined for Formula (III′).

In some embodiments, R₃′ is selected from chloro, fluoro, methyl, n-butylthio, n-butylamino, or —O—(C₆H₄)—OMe. In some embodiments, Z′ is selected from —CH₂—CH═CH₂, C₂-C₁₂ acyl, cyclohexyl, benzyl, —CH₂—(C₆H₄)—NO₂, —S(═O)₂—R₅′, and

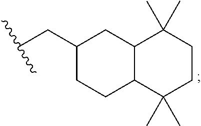

wherein R′₅ is selected from morpholinyl, 4-halophenyl, and 4-alkoxyphenyl. In some embodiments, both R₃′ and R₄′ are chloro.

In some embodiments, the compound of Formula (III′) is selected from the group comprising 4-((2,6-dichloro-7H-purin-7-yl)sulfonyl)morpholine, 4-((2,6-dichloro-9H-purin-9-yl)sulfonyl)morpholine, 2,6-dichloro-7-((4-fluorophenyl)sulfonyl)-7H-purine, 2,6-dichloro-9-((4-fluorophenyl)sulfonyl)-9H-purine, 2,6-dichloro-7-((4-methoxyphenyl)sulfonyl)-7H-purine, 2,6-dichloro-9-((4-methoxyphenyl)sulfonyl)-9H-purine, 2,6-dichloro-7-((5,5,8,8-tetramethyl-5,6,7,8-tetrahydronaphthalen-2-yl)methyl)-7H-purine, 2,6-dichloro-9-((5,5,8,8-tetramethyl-5,6,7,8-tetrahydronaphthalen-2-yl)methyl)-9H-purine, 2,6-dichloro-7-(4-nitrobenzyl)-7H-purine, 1-(2,6-dichloro-9H-purin-9-yl)dodecan-1-one, 1-(2,6-dichloro-7H-purin-7-yl)hexan-1-one, 1-(2,6-dichloro-9H-purin-9-yl)hexan-1-one, 1-(6-chloro-2-fluoro-7H-purin-7-yl)hexan-1-one, 1-(6-chloro-2-fluoro-9H-purin-9-yl)hexan-1-one, 1-(2-chloro-6-methyl-9H-purin-9-yl)hexan-1-one, 2-chloro-9-cyclohexyl-6-methyl-9H-purine, 9-cyclohexyl-2-fluoro-6-methyl-9H-purine, 1-(6-(butylthio)-2-fluoro-9H-purin-9-yl)ethan-1-one, 1-(6-(butylthio)-2-chloro-9H-purin-9-yl)ethan-1-one, 1-(6-(butylthio)-2-chloro-7H-purin-7-yl)ethan-1-one, 1-(6-(butylthio)-2-fluoro-7H-purin-7-yl)ethan-1-one, 1-(2-chloro-6-(p-tolyloxy)-9H-purin-9-yl)ethan-1-one, 1-(2-fluoro-6-(p-tolyloxy)-9H-purin-9-yl)ethan-1-one, 1-(2-chloro-6-(p-tolyloxy)-7H-purin-7-yl)ethan-1-one, 1-(2-fluoro-6-(p-tolyloxy)-7H-purin-7-yl)ethan-1-one, 1-(6-(butylamino)-2-chloro-7H-purin-7-yl)ethan-1-one, 1-(6-(butylamino)-2-fluoro-7H-purin-7-yl)ethan-1-one, 7-allyl-2,6-dichloro-7H-purine, 9-allyl-2,6-dichloro-9H-purine, 2,6-dichloro-7-benzyl-7H-purine, 2,6-dichloro-9-benzyl-9H-purine, 2,6-dichloro-7-(4-nitrobenzyl)-7H-purine, 2,6-dichloro-9-(4-nitrobenzyl-9H-purine, 2-(2,6-dichloro-9H-purin-9-yl)-5-(hydroxymethyl)tetrahydrofuran-3,4-diol, 1-(6-(butylamino)-2-chloro-9H-purin-9-yl)ethan-1-one, 1-(6-(butylamino)-2-fluoro-9H-purin-9-yl)ethan-1-one, 2,6-dichloro-N,N-diethyl-7H-purine-7-sulfonamide, 2,6-dichloro-N,N-diethyl-9H-purine-9-sulfonamide, N-benzyl-2,6-dichloro-N-methyl-9H-purine-9-sulfonamide, N-benzyl-2,6-dichloro-N-methyl-7H-purine-7H-sulfonamide, benzyl 2,6-dichloro-7H-purine-7-sulfonate, benzyl 2,6-dichloro-9H-purine-9-sulfonate, methyl 2,6-dichloro-9H-purine-9-sulfonate, and methyl 2,6-dichloro-7H-purine-7-sulfonate.

In some embodiments, modulating an activity of a protein comprising a reactive cysteine residue comprises inhibiting an activity of the protein comprising a reactive cysteine residue. In some embodiments, modulating an activity of a protein comprising a reactive cysteine residue comprises activating an activity of the protein comprising a reactive cysteine residue. In some embodiments, modulating an activity of a protein comprising a reactive cysteine residue comprises blocking a protein-protein interaction of the protein comprising a reactive cysteine residue. In some embodiments, modulating an activity of a protein comprising a reactive cysteine residue comprises disrupting a protein-RNA interaction of the protein comprising a reactive cysteine residue. In some embodiments, modulating an activity of a protein comprising a reactive cysteine residue comprises disrupting a protein-DNA interaction of the protein comprising a reactive cysteine residue. In some embodiments, modulating an activity of a protein comprising a reactive cysteine residue comprises disrupting a protein-lipid interaction of the protein comprising a reactive cysteine residue. In some embodiments, modulating the activity of a protein comprising a reactive cysteine residue comprises disrupting a protein-metabolite interaction of the protein comprising a reactive cysteine residue. In some embodiments, modulating an activity of a protein comprising a reactive cysteine residue comprises disrupting subcellular localization of the protein comprising a reactive cysteine residue. In some embodiments, modulating an activity of a protein comprising a reactive cysteine residue comprises triggering recruitment of an E3 ligase for targeted degradation of the protein comprising a reactive cysteine residue.

Accordingly, it is an object of the presently disclosed subject matter to provide methods of identifying reactive amino acid residues in proteins and methods of modulating the activity of proteins comprising reactive cysteine residues, as well as to provide covalent probes and related compounds and modified proteins. This and other objects are achieved in whole or in part by the presently disclosed subject matter.

An object of the presently disclosed subject matter having been stated above, other objects and advantages of the presently disclosed subject matter will become apparent to those of ordinary skill in the art after a study of the following description of the presently disclosed subject matter and non-limiting Figures and Examples.

### BRIEF DESCRIPTION OF THE FIGURES

FIG. 1A is a schematic diagram showing the design of an activity-based protein profiling (ABPP) probe for the investigation of enzymes, comprising an enzyme-recognition moiety attached to or substituted by a reactive group (i.e., a group that can react with an enzyme or other protein being investigated) and a tag (e.g., which can be reacted with a detectable moiety).

FIG. 1B is a schematic drawing of an exemplary general structure of purine-derived, activity-based protein profiling (ABPP) probes of the presently disclosed subject matter, where E groups, i.e., reactive/leaving groups, are substituted on the more electron-deficient pyrimidine ring of the purine scaffold and a tag is attached to the more electron-rich imidazole ring of the purine scaffold. The tag can comprise a detectable group or be a group that can be derivatized with a detectable group.

FIG. 1C is a schematic diagram showing the reaction of a nucleophilic group (Nu:) of a reactive amino acid residue of an enzyme (Enz) or other protein with an exemplary purine-based probe compound of the presently disclosed subject matter. Reaction with the nucleophilic group results in covalent modification of the enzyme or other protein and the loss of the chloro leaving group at carbon-6 of the purine-based probe. The propargyl group on the imidazole ring of the purine scaffold can be used for later derivatization of the modified enzyme or other protein with a detectable group (e.g., a fluorophore or specific binding partner). When the R group at carbon-2 of the purine-based probe is a potential leaving group (e.g., a halo atom), the nucleophilic group of the reactive amino acid residue of the enzyme can alternatively react to form a covalent bond at carbon-2.

FIG. 2A is a schematic diagram showing the synthesis of exemplary activity-based probes (ABPs) of the presently disclosed subject matter, i.e., 6-chloro-9-(prop-2-yn-1-yl)-9H-purine (Pu-4) and 6-chloro-7-(prop-2-yn-1-yl)-7H-purine (Pu-3). The major product of the reaction of 6-chloropurine and propargyl bromide is the N9-substituted purine.

FIG. 2B is a schematic diagram showing the structures of exemplary activity-based probes (ABP) of the presently disclosed subject matter comprising one or two chloro substituents on the pyrimidine ring and a propargyl group attached at one of the two nitrogen atoms of the imidazole group of the purine core structure. Ratios are provided for the major (N9-substituted) and minor (N7-substituted) products of the reactions of the chloro- or dichloropurine starting material with propargyl bromide.

FIG. 3A is a schematic drawing showing the workflow for a solution-based activity assay of purine probes with small molecule mimetics of amino acid residues, e.g., butanethiol. Analysis of the time course of the reaction is performed via high performance liquid chromatography (HPLC).

FIG. 3B is a series of graphs showing the solution-based reactivity (% starting material consumed versus time) of two exemplary probes, i.e., 2,6-dichloro-7-(prop-2-yn-1-yl)-7H-purine (AHL125 or Pu-1; data shown by “+”s) and 2,6-dichloro-9-(prop-2-yn-1-yl)-9H-purine (AHL128 or Pu-2, data shown by “x”s) for different small molecule amino acid residue mimetics. The graph at the top left is the reactivity data for the cysteine mimetic butanethiol; the graph at the top right is the reactivity data for the glutamine/asparagine mimetic propionamide; the graph at the middle left is the reactivity data for the aspartic acid/glutamic acid mimetic butyric acid; the graph at the middle right is the reactivity data for the tyrosine mimetic p-cresol; the graph at the bottom left is the reactivity data for the lysine mimetic butylamine.

FIG. 3C is a series of graphs showing the solution-based reactivity (% starting material consumed versus time) of ten exemplary probes of the presently disclosed subject matter for the cysteine residue mimetic butanethiol. The graph at the top left shows data for Pu-1 (filled circles) and Pu-2 (unfilled circles). The graph at the middle left shows data for Pu-3 (filled squares) and Pu-4 (unfilled squares). The graph at the bottom left shows data for Pu-5 (filled triangles) and Pu-6 (unfilled triangles). The graph at the top right shows data for Pu-7 (filled circles) and Pu-8 (unfilled circles). The graph at the middle right shows data for Pu-9 (filled circles) and Pu-10 (unfilled circles). The structures for the mono- and di-chloro purine-based probes Pu-1 to Pu-6 are shown in FIG. 2B. Pu-7 and Pu-8 are the 2-amino-6-chloro-7-(prop-2-yn-1-yl)-7H-purine and 2-amino-6-chloro-9-(prop-2-yn-1-yl)-9H-purine, respectively. Pu-9 and Pu-10 are 2,6-difluoro-7-(prop-2-yn-1-yl)-7H-purine and 2,6-difluoro-9-(prop-2-yn-1-yl)-9H-purine, respectively.

FIG. 3D is a graph comparing the solution-based reactivity (% starting material consumed versus time) of two exemplary purine-based protein ligands, 2,6-dichloro-7-benzyl-7H-purine (Pi-1, filled circles) and 2,6-dichloro-9-benzyl-9H-purine (Pi-2, unfilled circles) for the cysteine residue mimetic butanethiol.

FIG. 3E is a graph comparing the solution-based reactivity (% starting material consumed versus time) of two exemplary purine-based protein ligands, 7-allyl-2,6-dichloro-7H-purine (Pi-3, filled squares) and 9-allyl-2,6-dichloro-9H-purine (Pi-4, unfilled squares) for the cysteine residue mimetic butanethiol.

FIG. 3F is a graph comparing the solution-based reactivity (% starting material consumed versus time) of two exemplary purine-based protein probes, 6-butylthio-2-chloro-7-(prop-2-yn-1-yl)-7H-purine (Pa-1, data shown in circles) and 6-butylthio-2-chloro-9-(prop-2-yn-1-yl)-9H-purine (Pa-4, data shown in squares) for the cysteine residue mimetic butanethiol.

FIG. 3G is a graph comparing the solution-based reactivity (% starting material consumed versus time) of two exemplary purine-based protein probes, 2-butylthio-6-chloro-9-(prop-2-yn-1-yl)-9H-purine (Pa-3, data shown in triangles) and 6-butylthio-2-chloro-9-(prop-2-yn-1-yl)-9H-purine (Pa-4, data shown in squares) for the cysteine residue mimetic butanethiol.

FIG. 4A is a schematic diagram showing the workflow for an assay for the detection of reactive amino acid residues in a protein sample using a purine-derived activity-based probe (ABP) of the presently disclosed subject matter. Modification of proteins comprising reactive amino acid residues in a cell or cell lysate sample with the probe is followed by reaction of an alkyne group via copper-catalyzed azide-alkyne cycloaddition (CuAAC) to provide a triazole adduct conjugated to a detectable moiety for detection of the adduct via in-gel fluorescence detection using a sodium dodecyl sulfate polyacrylamide gel electrophoresis (SDS-PAGE) according to the presently disclosed subject matter.

FIG. 4B is a series of photographic images of fluorescent gel assays showing the activity of purine-based probes in live cells. The image at the left compares the activity of several exemplary probes, Pu-1 through Pu-10, the structures of which are described in FIG. 2B or the brief description for FIG. 3C. The image in the center shows the concentration dependence of the activity of Pu-1 (AHL125) and Pu-2 (AHL128). The image at the right shows the time dependence of the activity of Pu-1 and Pu-2.

FIG. 4C is a series of photographic images of fluorescent gel assays showing the activity of purine-based probes in cell lysates. The image at the left compares the activity of several exemplary probes, Pu-1 through Pu-10, the structures of which are described in FIG. 2B or the brief description for FIG. 3C. The image in the center shows the concentration dependence of the activity of Pu-1 (AHL125). The image at the right shows the time dependence of the activity of Pu-1 and Pu-2 (AHL128).

FIG. 5A is a composite image of a fluorescent gel showing the in vivo activity in lung tissue of three exemplary probes of the presently disclosed subject matter, 2,6-dichloro-7-(prop-2-yn-1-yl)-7H-purine (Pu-1), 2,6-dichloro-9-(prop-2-yn-1-yl)-9H-purine (Pu-2), and 2-chloro-7-(prop-2-yn-1-yl)-7H-purine (Pu-5), administered to mice via intraperitoneal injection (IP) or oral gavage (OG) at two different doses.

FIG. 5B is a composite image of a fluorescent gel showing the in vivo activity in liver tissue of three exemplary probes of the presently disclosed subject matter, 2,6-dichloro-7-(prop-2-yn-1-yl)-7H-purine (Pu-1), 2,6-dichloro-9-(prop-2-yn-1-yl)-9H-purine (Pu-2), and 2-chloro-7-(prop-2-yn-1-yl)-7H-purine (Pu-5), administered to mice via intraperitoneal injection (IP) or oral gavage (OG) at two different doses.

FIG. 5C is a composite image of a fluorescent gel showing the in vivo activity in spleen tissue of three exemplary probes of the presently disclosed subject matter, 2,6-dichloro-7-(prop-2-yn-1-yl)-7H-purine (Pu-1), 2,6-dichloro-9-(prop-2-yn-1-yl)-9H-purine (Pu-2), and 2-chloro-7-(prop-2-yn-1-yl)-7H-purine (Pu-5), administered to mice via intraperitoneal injection (IP) or oral gavage (OG) at two different doses.

FIG. 5D is a composite image of a fluorescent gel showing the in vivo activity in heart tissue of three exemplary probes of the presently disclosed subject matter, 2,6-dichloro-7-(prop-2-yn-1-yl)-7H-purine (Pu-1), 2,6-dichloro-9-(prop-2-yn-1-yl)-9H-purine (Pu-2), and 2-chloro-7-(prop-2-yn-1-yl)-7H-purine (Pu-5), administered to mice via intraperitoneal injection (IP) or oral gavage (OG) at two different doses.

FIG. 5E is a composite image of a fluorescent gel showing the in vivo activity in brain tissue of three exemplary probes of the presently disclosed subject matter, 2,6-dichloro-7-(prop-2-yn-1-yl)-7H-purine (Pu-1), 2,6-dichloro-9-(prop-2-yn-1-yl)-9H-purine (Pu-2), and 2-chloro-7-(prop-2-yn-1-yl)-7H-purine (Pu-5), administered to mice via intraperitoneal injection (IP) or oral gavage (OG) at two different doses.

FIG. 5F is a composite image of a fluorescent gel showing the in vivo activity in kidney tissue of three exemplary probes of the presently disclosed subject matter, 2,6-dichloro-7-(prop-2-yn-1-yl)-7H-purine (Pu-1), 2,6-dichloro-9-(prop-2-yn-1-yl)-9H-purine (Pu-2), and 2-chloro-7-(prop-2-yn-1-yl)-7H-purine (Pu-5), administered to mice via intraperitoneal injection (IP) or via oral gavage (OG) at two different doses in kidney tissue.

FIG. 5G is a composite image of a fluorescent gel showing the in vivo activity in white adipose tissue (WAT) of three exemplary probes of the presently disclosed subject matter, 2,6-dichloro-7-(prop-2-yn-1-yl)-7H-purine (Pu-1), 2,6-dichloro-9-(prop-2-yn-1-yl)-9H-purine (Pu-2), 2-chloro-7-(prop-2-yn-1-yl)-7H-purine (Pu-5), administered to mice via intraperitoneal injection (IP) or via oral gavage (OG) at two different doses.

FIG. 6 is a pair of photographic images of the fluorescent gel-based analysis of (left) the activity of 2,6-dichloro-7-(prop-2-yn-1-yl)-7H-purine (Pu-1) in vivo in mice in different tissues after two hours and (right) the activity of 2,6-dichloro-9-(prop-2-yn-1-yl)-9H-purine (Pu-2) in vivo in mice in different tissues after four hours.

FIG. 7 is a schematic diagram showing the workflow of an assay for the detection of reactive amino acid residues in a protein sample using a purine-derived activity-based probe (ABP) according to the presently disclosed subject matter. The work flow is the same as that shown in FIG. 4A, except that after formation of the triazole adduct, the modified proteins are digested with trypsin, the digested sample is enriched for the modified fragments, and the modified fragments are analyzed via liquid chromatography-tandem mass spectroscopy (LC-MS/MS).

FIG. 8 is a schematic diagram showing the workflow of a fluorescent gel-based competition assay using an activity-based probe (ABP) of the presently disclosed subject matter and a competitive inhibitor.

FIG. 9 is a schematic diagram showing the structures of exemplary purine-based ligands of the presently disclosed subject matter.

FIG. 10A is a schematic diagram of the four binding domains (D1, D2, D3, and D4) of selenocysteine elongation factor (eEF-Sec) and the binding site (Cysteine 442) of an exemplary probe of the presently disclosed subject matter, i.e., 2,6-dichloro-7-(prop-2-yn-1-yl)-7H-purine, also referred to herein as Pu-1, AHL-Pu-1, and AHL125.

FIG. 10B is a pair of photographic images of the fluorescent gel-based analysis of the time dependent reaction of (left) 2,6-dichloro-7-(prop-2-yn-1-yl)-7H-purine (AHL-Pu-1) or (right) 2,6-dichloro-9-(prop-2-yn-1-yl)-9H-purine (AHL-Pu-2) with wild-type selenocysteine elongation factor (eEF-Sec; WT) in live cells. The arrow pointing to the band at about 75 kilodalton (kDa) shows the band for probe-modified eEF-Sec. For comparison, the probes were contacted with cells overexpressing a mutant eEF-SEC where the cysteine at amino acid 442 is changed to alanine (Mutant) and cells not transfected with or overexpressing any eEF-Sec (Mock). Reaction times were varied from 0.5 to 2 hours.

FIG. 10C is a photographic image of the fluorescent gel-based analysis of the concentration dependence of the reaction of 2,6-dichloro-7-(prop-2-yn-1-yl)-7H-purine (AHL-Pu-1) with wild-type selenocysteine elongation factor (WT) or a mutant eEF-Sec where the cysteine at amino acid 442 is changed to alanine (CS) overexpressed in human embryonic kidney (HEK) cell proteomes. Reaction time was varied from 30 minutes to 120 minutes. The arrow points to the band for the probe-modified eEF-Sec at about 75 kilodaltons. The concentration of AHL-Pu-1 was varied from 1 micromolar (μM) to 50 μM. “Mock” refers to cells not transfected with WT or mutant eEF-Sec.

FIG. 10D is a photographic image of the fluorescent gel-based analysis of a competition assay where purine ligands (2,6-dichloro-7-(4-nitrobenzyl)-7H-purine (Pi-5), 4-((2,6-dichloro-9H-purin-9-yl)sulfonyl)morpholine (Pi-8), or 2,6-dichloro-7-benzyl-7H-purine (Pi-1); 4 hr treatment) block 2,6-dichloro-7-(prop-2-yn-1-yl)-7H-purine (AHL-Pu-1) probe labeling (25 μM; 1 hr) of selenocysteine elongation factor (eEF-Sec) in a concentration dependent manner in live cells. Concentrations of the purine ligands was varied from 0.5 micromolar (μM) to 25 μM.

FIG. 10E is a photographic image of the fluorescent gel-based analysis of a competition assay where purine ligands (2,6-dichloro-7-(4-nitrobenzyl)-7H-purine (Pi-5) or 2,6-dichloro-7-benzyl-7H-purine (Pi-1); 4 hr treatment) block 2,6-dichloro-7-(prop-2-yn-1-yl)-7H-purine (AHL-Pu-1) probe labeling (25 μM; 1 hr) of selenocysteine elongation factor (eEF-Sec) in a concentration dependent manner in live cells. Concentrations of the purine ligands was varied from 0.05 micromolar (μM) to 25 μM.

FIG. 11 is a graph showing the functional protein domains that are statistically significantly enriched by Pu-1 and Pu-2 purine probes.

FIG. 12 is a graph showing the subcellular location analysis of Pu-1- and Pu-2-modified proteins from live cell studies.

### DETAILED DESCRIPTION

The presently disclosed subject matter will now be described more fully hereinafter with reference to the accompanying Figures and Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art. Certain components in the figures are not necessarily to scale, emphasis instead being placed upon illustrating the principles of the presently disclosed subject matter (in some cases schematically).

Unless otherwise defined, all technical and scientific terms used herein have the same meaning as commonly understood by one of ordinary skill in the art to which the presently described subject matter belongs. All publications, patent applications, patents, and other references mentioned herein are incorporated by reference in their entirety.

Throughout the specification and claims, a given chemical formula or name shall encompass all active optical and stereoisomers, as well as racemic mixtures where such isomers and mixtures exist.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

References to techniques employed herein are intended to refer to the techniques as commonly understood in the art, including variations on those techniques or substitutions of equivalent techniques that would be apparent to one of skill in the art.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “a protein” refers to one or more proteins, including a plurality of the same protein. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, to including but not limited to skin, hair, tissue, blood, plasma, cells, sweat, and urine.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is leukemia, which in some embodiments is Acute Myeloid Leukemia (AML).

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

In some embodiments, the terms “fragment”, “segment”, or “subsequence” as used herein refers to a portion of an amino acid sequence, comprising at least one amino acid, or a portion of a nucleic acid sequence comprising at least one nucleotide. Thus, in some embodiments, the terms “fragment”, “segment”, and “subsequence” are used interchangeably herein. In some embodiments, the term “fragment” refers to a compound (e.g., a small molecule compound, such as a small molecule comprising a purine scaffold) that can react with a reactive amino acid residue (e.g., a reactive cysteine) to form an adduct comprising a modified amino acid residue. Thus, in some embodiments, the terms “fragment” and “ligand” are used interchangeably. In some embodiments, the term “fragment” refers to that portion of a ligand that remains covalently attached to the reactive amino acid residue.

As used herein, a “ligand” is a compound (e.g., a purine-based compound) that specifically binds to a target compound or molecule, such as a reactive nucleophilic amino acid residue in a protein. In some embodiments, the ligand can bind to the target covalently. A ligand “specifically binds to” or “is specifically reactive with” a compound (e.g., a reactive amino acid residue) when the ligand functions in a binding reaction which is determinative of the presence of the compound in a sample of heterogeneous compounds.

As used herein, a “functional” biological molecule is a biological molecule in a form in which it exhibits a property by which it can be characterized. A functional enzyme, for example, is one that exhibits the characteristic catalytic activity by which the enzyme can be characterized.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

As used herein, the term “mass spectrometry” (MS) refers to a technique for the identification and/or quantitation of molecules in a sample. MS includes ionizing the molecules in a sample, forming charged molecules; separating the charged molecules according to their mass-to-charge ratio; and detecting the charged molecules. MS allows for both the qualitative and quantitative detection of molecules in a sample. The molecules can be ionized and detected by any suitable means known to one of skill in the art. Some examples of mass spectrometry are “tandem mass spectrometry” or “MS/MS,” which are the techniques wherein multiple rounds of mass spectrometry occur, either simultaneously using more than one mass analyzer or sequentially using a single mass analyzer. The term “mass spectrometry” can refer to the application of mass spectrometry to protein analysis. In some embodiments, electrospray ionization (ESI) and matrix-assisted laser desorption/ionization (MALDI) can be used in this context. In some embodiments, intact protein molecules can be ionized by the above techniques, and then introduced to a mass analyzer. Alternatively, protein molecules can be broken down into smaller peptides, for example, by enzymatic digestion by a protease, such as trypsin. Subsequently, the peptides are introduced into the mass spectrometer and identified by peptide mass fingerprinting or tandem mass spectrometry.

As used herein, the term “mass spectrometer” is used to refer an apparatus for performing mass spectrometry that includes a component for ionizing molecules and detecting charged molecules. Various types of mass spectrometers can be employed in the methods of the presently disclosed subject matter. For example, whole protein mass spectroscopy analysis can be conducted using time-of-flight (TOF) or Fourier transform ion cyclotron resonance (FT-ICR) instruments. For peptide mass analysis, MALDI time-of-flight instruments can be employed, as they permit the acquisition of peptide mass fingerprints (PMFs) at high pace. Multiple stage quadrupole-time-of-flight and the quadrupole ion trap instruments can also be used.

The terms “high throughput protein identification,” “proteomics” and other related terms are used herein to refer to the processes of identification of a large number or (in some cases, all) proteins in a certain protein complement. Post-translational protein modifications and quantitative information can also be assessed by such methods. One example of “high throughput protein identification” is a gel-based process that includes the pre-fractionation and purification of proteins by one-dimensional protein gel electrophoresis. The gel can then be fractionated into several molecular weight fractions to reduce sample complexity, and proteins can be in-gel digested with trypsin. The tryptic peptides are extracted from the gel, further fractionated by liquid chromatography and analyzed by mass spectrometry. In another approach, a sample can be fractionated without using the gels, for example, by protein extraction followed by liquid chromatography. The proteins can then be digested in-solution, and the proteolytic fragments further fractionated by liquid chromatography and analyzed by mass spectrometry.

As used herein, the term “Western blot,” which can be also referred to as “immunoblot”, and related terms refer to an analytical technique used to detect specific proteins in a sample. The technique uses gel electrophoresis to separate the proteins, which are then transferred from the gel to a membrane (typically nitrocellulose or PVDF) and stained, in membrane, with antibodies specific to the target protein.

The expression “stable isotope labeling by amino acids in cell culture” (SILAC) is used herein to refer to an approach for incorporation of a label into proteins for mass spectrometry (MS)-based quantitative proteomics. SILAC comprises metabolic incorporation of a given “light” or “heavy” form of the amino acid into the proteins. For example, SILAC comprises the incorporation of amino acids with substituted stable isotopic nuclei (e.g. deuterium, ¹³C, ¹⁵N). In an illustrative SILAC experiment, two cell populations are grown in culture media that are identical, except that one of them contains a “light” and the other a “heavy” form of a particular amino acid (for example, ¹²C and ¹³C labeled L-lysine, respectively). When the labeled analog of an amino acid is supplied to cells in culture instead of the natural amino acid, it is incorporated into all newly synthesized proteins. After a number of cell divisions, each instance of the amino acid is replaced by its isotope-labeled analog. Since there is little chemical difference between the labeled amino acid and the natural amino acid isotopes, the cells behave substantially similar to the control cell population grown in the presence of a normal amino acid.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains proteins, cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

As used herein the term “alkyl” refers to C₁-20 inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. In some embodiments, the alkyl group is “lower alkyl.” “Lower alkyl” refers to an alkyl group having 1 to about 8 carbon atoms (i.e., a C₁₋₈ alkyl), e.g., 1, 2, 3, 4, 5, 6, 7, or 8 carbon atoms. In some embodiments, the alkyl is “higher alkyl.” “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic moiety that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, carbonyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “heteroaryl” refers to aryl groups wherein at least one atom of the backbone of the aromatic ring or rings is an atom other than carbon. Thus, heteroaryl groups have one or more non-carbon atoms selected from the group including, but not limited to, nitrogen, oxygen, and sulfur.

As used herein, the term “acyl” refers to an organic carboxylic acid group wherein the —OH of the carboxyl group has been replaced with another substituent (i.e., as represented by RC(═O)—, wherein R is an alkyl, substituted alkyl, aralkyl, substituted aralkyl, aryl or substituted aryl group as defined herein). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The terms “heterocycle”, “heterocyclyl” “heterocycloalkyl” or “heterocyclic” refer to cycloalkyl groups (i.e., non-aromatic, cyclic groups as described hereinabove) wherein one or more of the backbone carbon atoms of a cyclic ring is replaced by a heteroatom (e.g., nitrogen, sulfur, or oxygen). Examples of heterocycles include, but are not limited to, tetrahydrofuran, tetrahydropyran, morpholine, dioxane, piperidine, piperazine, and pyrrolidine. Additional examples of heterocycles include, for example, the cyclic forms of sugars, such as ribose, glucose, galactose, and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described.

Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Alkoxyl” or “alkoxy” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangably with “alkoxyl”.

The terms “aryloxy” and “aryloxyl” refer to an aryl-O-group, wherein aryl is as previously described. The term “aryloxy as used herein can refer to, for example, phenoxy, p-chlorophenoxy, p-fluorophenoxy, p-methylphenoxy, p-methoxyphenoxy, and the like.

“Aralkyl” refers to an aryl-alkyl-group wherein aryl and alkyl are as previously described and include substituted aryl and substituted alkyl. Exemplary aralkyl groups include benzyl, phenylethyl, and naphthylmethyl. In some embodiments, the aromatic portion of the aralkyl group can be substituted by one or more aryl group substituents and/or the alkyl portion of the aralkyl group can be substituted by one or more alkyl group substituents and the aralkyl group can be a “substituted aralkyl” group.

The term “amino” refers to the —NR′R″ group, wherein R′ and R″ are each independently selected from the group including H and substituted and unsubstituted alkyl, cycloalkyl, heterocycle, aralkyl, aryl, and heteroaryl. In some embodiments, the amino group is —NH₂.

The terms “alkylamino” and “aminoalkyl” refer to a —NHR group where R is alkyl or substituted alkyl. The term “arylamino” refers to a —NHR group where R is aryl or substituted aryl.

The term “carbonyl” refers to the —(C═O)— or a double bonded oxygen substituent attached to a carbon atom of a previously named parent group.

The terms “carboxylate” and “carboxylic acid” can refer to the groups —C(═O)—O— and —C(═O)—OH, respectively. In some embodiments, “carboxylate” can refer to either the —C(═O)—O⁻ or —C(═O)—OH group. In some embodiments, the term “carboxyl” can also be used to refer to a carboxylate or carboxylic acid group.

The terms “sulfonyl”, “sulfone”, and “sulphone” as used herein refer to the —S(═O)₂— or —S(═O)₂R group, wherein R is alkyl, substituted alkyl, cycloalkyl, heterocycloalkyl, aralkyl, substituted aralkyl, aryl, substituted aryl, heteroaryl, or substituted heteroaryl.

The term “sulfonamide” refers to the —S(═O)₂—N(R)₂ group, wherein each R is independently selected from H, alkyl, substituted alkyl, aralkyl, substituted aralkyl, aryl, and substituted aryl, or wherein the two R together can for a ring with the nitrogen atom (e.g., wherein the two R are together an alkylene group, such as a butylene or pentylene group).

The term “sulfonate” as used herein refers to a —S(═O)₂—O—R group, wherein R is selected from alkyl, substituted alkyl, aralkyl, substituted aralkyl, aryl, and substituted aryl.

The terms “halo”, “halide”, or “halogen” as used herein refer to fluoro, chloro, bromo, and iodo groups.

The term “perhaloalkyl” refers to an alkyl group wherein all of the hydrogen atoms are replaced by halo. Thus, for example, perhaloalkyl can refer to a “perfluroalkyl” group wherein all of the hydrogen atoms of the alkyl group are replaced by fluoro. Perhaloalkyl groups include, but are not limited to, —CF₃.

The terms “hydroxyl” and “hydroxy” refer to the —OH group.

The term “oxo” refers to a compound described previously herein wherein a carbon atom is replaced by an oxygen atom.

The term “thio” refers to the —S— or —SH group.

The terms “alkylthio” and “thioalkyl” refer to a —SR group where R is alkyl or substituted alkyl. The term “arylthiol” refers to a —SR group where R is aryl or substituted aryl.

The term “cyano” refers to the —CN group.

The term “nitro” refers to the —NO₂ group.

A line crossed by a wavy line, e.g., in the structure:

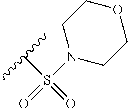 [SMILES: *S(=O)(=O)N1CCOCC1]

indicates the site where the indicated substituent can bond to another group.

### II. General Considerations

Covalent probes serve as invaluable tools for the global investigation of protein function and ligand binding capacity. While several probes have been deployed for the interrogation of nucleophilic residues such as cysteine (IA-Alkyne), lysine (NaTFBS-Alkyne), and methionine, a large fraction of the human proteome still remains inaccessible to pharmacological modulation.

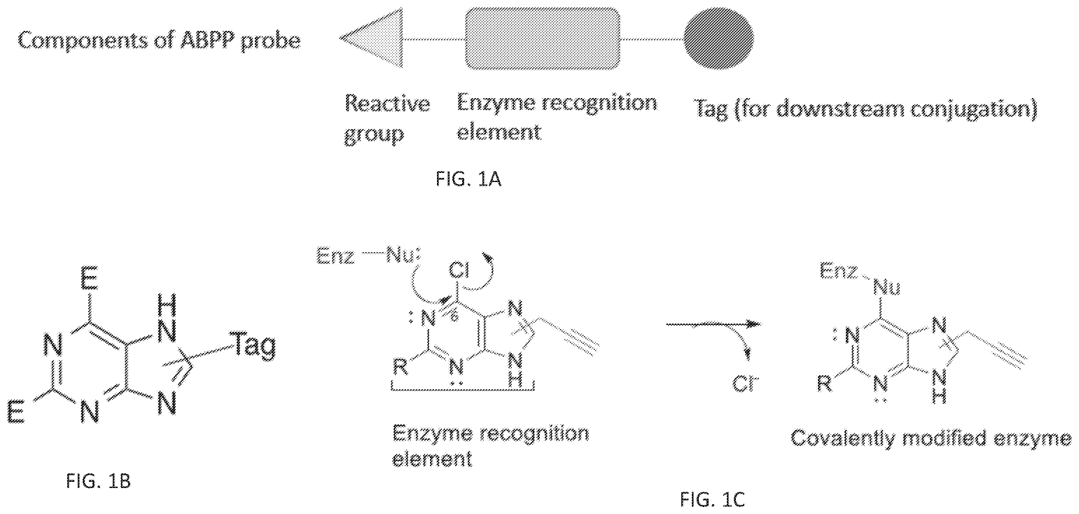

Activity-based protein profiling (ABPP) utilizes active-site directed chemical probes to measure the functional state of large numbers of enzymes in native biological systems (e.g. cells or tissues). Activity-based probes consist of a reactive group for targeting a specific enzyme class and a reporter tag for detection by in-gel fluorescence scanning or by avidin-enrichment coupled with liquid chromatography mass spectrometry (LCMS), respectively. See FIG. 1A. For ligand (e.g., inhibitor) discovery, the potency and selectivity of small molecules can be profiled against many enzymes in parallel by performing competitive ABPP in complex proteomes, where ligands compete for probe labeling of enzyme targets.

Purines are essential components of DNA and RNA and have been fine-tuned by nature for biological activity. Purines have historically been explored as a scaffold for the development of inhibitors but their application in chemical biology as probes to discover new target proteins and druggable sites has been limited. In one aspect, the presently disclosed subject matter relates to purine-derived chemical probes and their use as chemoproteomic tools for activity-based profiling of the proteome (e.g., the human proteome).

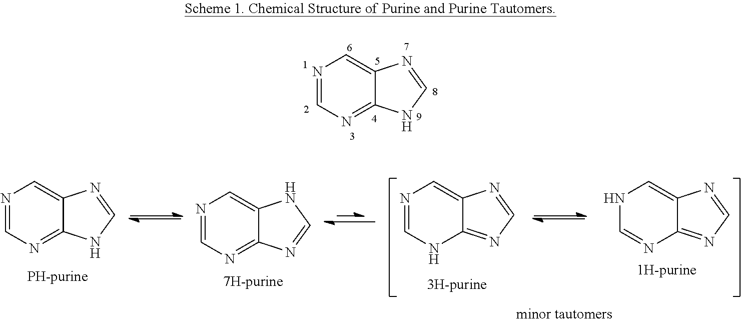

The chemical structure of purine with the atoms numbered, is shown at the top of Scheme 1, above. Scheme 1 further shows structures of the four purine tautomers, the main two tautomers, i.e., 9H-purine and 7H-purine, and the two minor tautomers, i.e., 3H-purine and 1H-purine. Chemically, the reactions of purine reflect the interplay between the constituent pyrimidine and imidazole rings of the purine scaffold. The general structure of purine-based probes of the presently disclosed subject matter is shown in FIG. 1B. Electron localization within the nitrogen atoms of the pyrimidine ring can render the C2 and particularly the C6 site amenable to nucleophilic attack by protein residues. Thus, one aspect of the presently disclosed purine-based probes (and ligands) is the addition of an electrophilic “warhead” (“E” in the structure of FIG. 1B) on the pyrimidine ring that can serve as an effective leaving group during nucleophilic attack by a nucleophilic group on a side chain of a protein residue. Meanwhile, the more electron rich imidazole ring can provide for facile derivatization, e.g., to attach detectable tags or taggable groups. Alternatively, the electron rich imidazole ring can serve for derivatization of the purine scaffold to provide a wide variety of covalent protein modulators (e.g., inhibitors or activators), also referred to herein as “ligands”. FIG. 1C shows the mechanism whereby a covalent enzyme/probe adduct is formed when the probe is contacted with an enzyme having a reactive nucleophilic residue. After formation (e.g., in a cell lysate, a live cell, a tissue, a living organism, or another sample comprising one or more proteins), the covalent enzyme/probe adduct can be analyzed in-gel and/or by LC-MS/MS. The presently disclosed probes and related ligands/modulators can be used for target protein discovery, competitive ABPP, and inverse drug discovery.

In some embodiments, the presently disclosed subject matter provides small molecule probes that interact with reactive nucleophilic residues on proteins or peptides, such as a reactive cysteine residue of a cysteine-containing protein, as well as methods of identifying a protein or peptide that contains such a reactive residue (e.g., a druggable cysteine residue). In some instances, also described herein, are methods of profiling a small molecule purine-based ligand that interacts with one or more cysteine-containing protein comprising one or more reactive cysteine.

In some embodiments, the presently disclosed subject matter provides a method for identifying a reactive amino acid residue of a protein. In some embodiments, the method comprises: (a) providing a protein sample; (b) contacting the protein sample with a purine-based probe compound (e.g., a halo-substituted purine-based probe compound) for a period of time sufficient for the probe compound to react with at least one reactive amino acid in the protein sample, thereby forming at least one modified amino acid residue; and (c) analyzing proteins in or from the protein sample to identify the at least one modified amino acid residue, thereby identifying at least one reactive amino acid residue of a protein. In some embodiments, the protein sample comprises isolated proteins, living cells, a cell lysate or a biological organism (e.g., a mammal or other animal, a plant, a bacteria, etc.). In some embodiments, the probe compound has a structure of Formula (I):

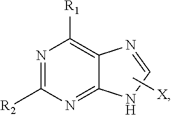

wherein: X is a monovalent moiety comprising an alkyne moiety, a fluorophore moiety, a detectable labeling group, or a combination thereof; and R₁ and R₂ are independently selected from the group comprising H, halo, amino, alkyl (e.g., C₁-C₆ alkyl), alkoxy (e.g., C₁-C₆ alkoxy), alkylthio (e.g., C₁-C₆ alkylthio), alkylamino (e.g., C₁-C₆ alkylamino), aryloxy, arylthiol, and arylamino, subject to the proviso that at least one of R₁ and R₂ is halo. In some embodiments, R₁ and R₂ are independently selected from H, halo and amino, subject to the proviso that at least one of R₁ and R₂ is halo. In some embodiments, at least one of R₁ and R₂ is chloro or fluoro.

In some embodiments, X is attached at one of the nitrogen atoms of the imidazole ring (i.e., N9 or N7). Thus, in some embodiments, the probe compound of Formula (I) has a structure of Formula (Ia):

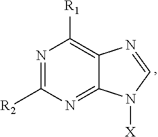

or a structure of Formula (Ib):

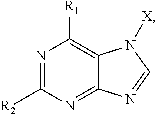

wherein X is a monovalent moiety comprising an alkyne moiety, a fluorophore moiety, a detectable labeling group, or a combination thereof; and R₁ and R₂ are independently selected from the group comprising H, halo, amino, alkyl, alkoxy, alkylthio, alkylamino, aryloxy, arylthiol, and arylamino, subject to the proviso that at least one of R₁ and R₂ is halo. In some embodiments, R₁ and R₂ are independently selected from the group consisting of H, halo, and amino, subject to the proviso that at least one of R₁ and R₂ is halo.

In some embodiments, the reactive amino acid residue is selected from the group comprising cysteine, lysine, glutamic acid, arginine, aspartic acid, glutamine, tyrosine, histidine, asparagine, methionine, threonine, tryptophan, and serine. In some embodiments, the reactive amino acid residue is selected from cysteine, lysine, glutamic acid, arginine, and aspartic acid. In some embodiments, the reactive amino acid residue is selected from cysteine, aspartic acid, glutamic acid, tyrosine, lysine, and glutamine. In some embodiments, the reactive amino acid residue is cysteine.

In some embodiments, the reactive amino acid residue is cysteine and the modified amino acid residue has a structure of Formula (IIa-i):

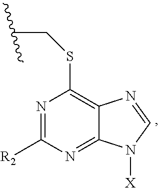

a structure of Formula (IIb-i):

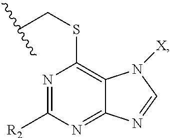

a structure of Formula (IIa-ii):

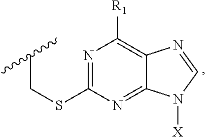

or a structure of Formula (IIb-ii):

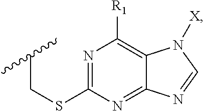

wherein X is a monovalent moiety comprising an alkyne moiety, a fluorophore moiety, a detectable labeling group, or a combination thereof; R₁ is selected from the group comprising H, halo, amino, alkyl, alkyoxy, alkylthio, alkylamino, aryloxy, arylthio, and arylamino; and R₂ is selected from the group comprising H, halo, amino, alkyl, alkoxy, alkylthio, alkylamino, aryloxy, arylthiol, and arylamino. In some embodiments, R₁ is H, halo or amino. In some embodiments, R₂ is H, halo or amino. In some embodiments, the modified amino acid residue has a structure of Formula (IIa-i) or Formula (IIb-i). In some embodiments, the modified amino acid residue has a structure of Formula (IIb-i).

In some embodiments, the compound of Formula (I), (Ia), or (Ib) is a compound where R₁ is halo. In some embodiments, R₁ is chloro or fluoro. In some embodiments, R₁ is chloro.

In some embodiments, the compound of Formula (I), (Ia), or (Ib) is a compound where R₂ is halo. In some embodiments, R₂ is chloro or fluoro. In some embodiments, R₂ is chloro.

In some embodiments, both of R₁ and R₂ are halo. In some embodiments, R₁ and R₂ are each chloro. In some embodiments, R₁ and R₂ are each fluoro. In some embodiments, R₁ is chloro and R₂ is fluoro.

In some embodiments, X comprises a fluorophore or a detectable labeling group such as described hereinbelow. In some embodiments, X is a monovalent moiety comprising an alkyne group (i.e., a carbon-carbon triple bond). For example, in some embodiments, X comprises or consists of —C≡CH, -alkylene-C≡CH, —C(═O)-alkylene-C≡CH, or —C(═O)—NH-alkylene-C≡CH (e.g., C(═O)—NH—CH₂—C≡CH). In some embodiments, the alkylene group is a C₁-C₅ alkylene group. In some embodiments, the alkylene group is methylene. In some embodiments, X is a propargyl group, i.e., —CH₂—C≡CH.

In some embodiments, the probe compound is selected from the group comprising 2,6-dichloro-7-(prop-2-yn-1-yl)-7H-purine (also referred to herein as AHL125, AHL-Pu-1, or Pu-1), 2,6-dichloro-9-(prop-2-yn-1-yl)-9H-purine (also referred to herein as AHL128, AHL-Pu-2, or Pu-2), 6-chloro-7-(prop-2-yn-1-yl)-7H-purine (also referred to herein as AHL-Pu-3 or Pu-3), 6-chloro-9-(prop-2-yn-1-yl)-9H-purine (also referred to herein as AHL-Pu-4 or Pu-4), 2-chloro-7-(prop-2-yn-1-yl)-7H-purine (also referred to herein as AHL-Pu-5 or Pu-5), 2-chloro-9-(prop-2-yn-1-yl)-9H-purine (also referred to herein as AHL-Pu-6 or Pu-6), 2,6-difluoro-7-(prop-2-yn-1-yl)-7H-purine (also referred to herein as AHL-Pu-11 or Pu-11), 2,6,-difluoro-9-(prop-2-yn-1-yl)-9H-purine (also referred to herein as AHL-Pu-12 or Pu-12), 6-chloro-2-fluoro-7-(prop-2-yn-1-yl)-7H-purine (also referred to herein as AHL-Pu-9 or Pu-9), 6-chloro-2-fluoro-9-(prop-2-yn-1-yl)-9H-purine (also referred to herein as AHL-Pu-10 or Pu-10), 6-chloro-2-amino-7-(prop-2-yn-1-yl)-7H-purine (also referred to herein as AHL-Pu-7 or Pu-7), and 6-chloro-2-amino-9-(prop-2-yn-1-yl)-9H-purine (also referred to herein as AHL-Pu-8 or Pu-8). In some embodiments, the probe is selected from Pu-1, Pu-2, Pu-9, and Pu-10.

In some embodiments, the N7-substituted probe is more reactive. Thus, in some embodiments, the probe compound has a structure of Formula (Ib), as shown above. In some embodiments, the purity of the probe compound having a structure of Formula (Ib) is about 90% or more (e.g., about 90, 91, 92, 93, 94, 95, 96, 97, 98, or about 99% or more), e.g., by HPLC. Thus, the N7-substituted probe can be provided substantially as a single regioisomer. In some embodiments, the probe compound is 2,6-dichloro-7-(prop-2-yn-1-yl)-7H-purine, 6-chloro-2-fluoro(7-prop-2-yn-1-yl)-7H-purine or 2,6-difluoro-7-(prop-2-yn-1-yl)-7H-purine. In some embodiments, the probe compound is 2,6-dichloro-7-(prop-2-yn-1-yl)-7H-purine or 6-chloro-2-fluoro(7-prop-2-yn-1-yl)-7H-purine. In some embodiments, the probe compound is 2,6-dichloro-7-(prop-2-yn-1-yl)-7H-purine.

In some embodiments, one of R₁ and R₂ is halo (e.g., chloro or fluoro) and the other of R₁ and R₂ is alkoxy, alkylthio or alkylamino. In some embodiments, one of R₁ and R₂ is alkylthio and the other of R₁ and R₂ is chloro or fluoro. Thus, in some embodiments, the probe compound is a “adduct” compound (i.e., a probe compound where one of R₁ and R₂ is replaced by a group from a small molecule amino acid mimetic), such as shown in Scheme 8, below in Example 4. In some embodiments, the compound is selected from the group comprising 6-(butylthio)-2-chloro-7-(prop-2-yn-1-yl)-7H-purine (Pa-1), 2-(butylthio)-6-chloro-7-(prop-2-yn-1-yl)-7H-purine (Pa-2), 2-(butylthio)-6-chloro-9-(prop-2-yn-1-yl)-9H-purine (Pa-3), 6-(butylthio)-2-chloro-9-(prop-2-yn-1-yl)-9H-purine (Pa-4), 6-(butylthio)-2-fluoro-7-(prop-2-yn-1-yl)-7H-purine (Pa-5), and 6-(butylthio)-2-fluoro-9-(prop-2-yn-1-yl)-9H-purine (Pa-6). In some embodiments, the compound is Pa-3, Pa-4, or Pa-6.

In some embodiments, e.g., when X comprises an alkyne group, the analyzing of step (c) further comprises tagging the at least one modified reactive amino acid (e.g., cysteine) residue with a compound comprising detectable labeling group, thereby forming at least one tagged reactive amino acid (e.g., cysteine) residue comprising said detectable labeling group. In some embodiments, the detectable labeling group comprises biotin or a biotin derivative. In some embodiments, the biotin derivative is desthiobiotin.

In some embodiments, the tagging comprises reacting an alkyne group in a X moiety of at least one modified reactive amino acid (e.g., cysteine) residue with a compound comprising both an azide moiety (or other alkyne-reactive group) and a detectable labeling group (e.g., biotin or a biotin derivative). In some embodiments, the compound comprising the azide moiety and the detectable labeling group further comprises an alkylene linker, which in some embodiments, can comprise a polyether group, such as an oligomer of methylene glycol, ethylene glycol, or propylene glycol (e.g., a group having the formula —(O—C₂H₄—)ₓ—). In some embodiments, the tagging comprises performing a copper-catalyzed azide-alkyne cycloaddition (CuAAC) coupling reaction.

In some embodiments, the analyzing further comprises digesting the protein sample to provide a digested protein sample comprising a protein fragment comprising the at least one tagged reactive amino acid residue (e.g., cysteine residue) moiety comprising the detectable group. In some embodiments, the digesting is performed with a peptidase. In some embodiments, the digesting is performed with trypsin.

In some embodiments, the analyzing further comprises enriching the digested protein sample for the detectable labeling group. For example, in some embodiments, the enriching comprises contacting the digested protein sample with a solid support comprising a binding partner of the detectable labeling group. In some embodiments, when the detectable labeling group comprises biotin or a derivative thereof, the solid support comprises streptavidin. In some embodiments, the analyzing further comprises analyzing the digested protein sample (e.g., the enriched digested protein sample) via liquid chromatography-mass spectrometry or via a gel-based assay.

In some embodiments, the protein sample is a biological organism and the presently disclosed method can be used to detect reactive amino acid residues of proteins in vivo. When the protein sample is a biological organism (i.e., a living biological organism), such as an animal, contacting the protein sample with the probe compound of Formula (I) comprises administering the probe compound of Formula (I) to the biological organism via a suitable route of administration. The administration can be systemic or localized (e.g., to a site of disease, such as a tumor). In some embodiments, the administration is oral administration or injection, e.g., i.v. or i.p. injection. In some embodiments, prior to analyzing the proteins, a tissue sample is removed from the biological organism and homogenized. Alternatively, a biological fluid sample (e.g., blood or saliva) can be collected and the proteins therein can be analyzed for detection of a modified amino acid residue.

In some embodiments, providing the protein sample further comprises separating the protein sample (e.g., a cell or cell lysate sample) into a first protein sample and a second protein sample. Then, in the contacting step, the first protein sample can be contacted with a first probe compound of Formula (I) at a first probe concentration for a first period of time and the second protein sample can be contacted with a second probe compound of Formula (I) (i.e., a probe compound of Formula (I) having a different structure than that of the first probe compound of Formula (I)) at the same probe concentration (i.e., at the first probe concentration) for the same time period (i.e., for the first period of time. Alternatively, the second protein sample can be contacted with the same probe compound as the first protein sample, but at a different probe concentration (i.e., a second probe concentration) or for a different period of time. In some embodiments, analyzing proteins comprises analyzing the first and second protein samples to determine the presence and/or identity of a modified reactive amino acid residue (e.g., a modified reactive cysteine residue) in the first sample and the presence and/or identity of a modified reactive amino acid residue (e.g., a modified reactive cysteine residue) in the second sample. In some embodiments, the identities and/or amounts of identified modified reactive amino acid residues (e.g., the modified reactive cysteine residues) from the first and second protein samples are compared.

In some embodiments, the protein sample comprises living cells. In some embodiments, providing the protein sample further comprises separating the protein sample into a first protein sample and a second protein sample and culturing the first protein sample in a first cell culture medium comprising heavy isotopes prior to the contacting of step (b) and culturing the second protein sample in a second cell culture medium, wherein the second culture medium comprises a naturally occurring isotope distribution prior to the contacting of step (b). In some embodiments, the first cell culture medium comprises ¹³C- and/or ¹⁵N-labeled amino acids. In some embodiments, the first cell culture medium comprises ¹³C-,¹⁵N-labeled lysine and arginine.

In some embodiments, e.g., if the protein sample does not comprise living cells, the probe compound of Formula (I) can comprise a detectable labeling group comprising a heavy isotope (e.g., a ¹³C label) or the method can comprise tagging the at least one modified amino acid residue with a detectable labeling group comprising a heavy isotope.

In some embodiments, the protein sample is separated into a first and a second protein sample and one of the first and the second protein sample is cultured in the presence of a compound or biomolecule that interacts with a protein present in or suspected of being present in the protein sample. In some embodiments, the compound or biomolecule that interacts with a protein present in or suspected of being present in the protein sample is an inhibitor or activator of an enzyme present in or suspected of being present in the protein sample. In some embodiments, one of the first and the second protein sample can be cultured in the presence of a ligand of the presently disclosed subject matter.

### III. Probes

In some embodiments, the presently disclosed subject matter provides a purine-based probe compound that comprises an electrophilic moiety (e.g., attached to a carbon on the pyrimidine ring of a purine scaffold) that can be displaced by a nucleophilic group in a side chain of an amino acid residue of a protein. The purine-based probe can also comprise a detectable group or a group (e.g., an alkyne group) that can be derivatized with a detectable group (e.g., a fluorophore or an antigen). In some embodiments, the purine-based probe reacts with a cysteine residue or other nucleophilic amino acid residue to form a covalent bond (e.g., a thio ether). Typically, the probe is a non-naturally occurring molecule, or forms a non-naturally occurring product (i.e., a “modified” protein) after reaction with the nucleophilic amino acid residue.

In some embodiments, the purine-based probe compound is a compound of one of Formulas (I), (Ia) or (Ib). Thus, in some embodiments, the probe compound has a structure of Formula (I):

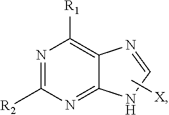

wherein X is a monovalent moiety comprising an alkyne moiety, a fluorophore moiety, a detectable labeling group, or a combination thereof; and R₁ and R₂ are independently selected from the group comprising H, halo, amino, alkyl (e.g., C₁-C₆ alkyl) alkoxy (e.g., C₁-C₆ alkoxy), alkylthio (e.g., C₁-C₆ alkylthio), alkylamino (e.g., C₁-C₆ alkylamino), aryloxy, arylthiol, and arylamino, subject to the proviso that at least one of R₁ and R₂ is halo. In some embodiments, R₁ and R₂ are independently selected from H, halo and amino, subject to the proviso that at least one of R₁ and R₂ is halo. In some embodiments, at least one of R₁ and R₂ is chloro or fluoro.

In some embodiments, X comprises a fluorophore or a detectable labeling group. The fluorophore of X can be any suitable fluorophore. In some embodiments, the fluorophore is selected from the group including, but not limited to, rhodamine, rhodol, fluorescein, thiofluorescein, aminofluorescein, carboxyfluorescein, chlorofluorescein, methylfluorescein, sulfofluorescein, aminorhodol, carboxyrhodol, chlororhodol, methylrhodol, sulforhodol; aminorhodamine, carboxyrhodamine, chlororhodamine, methylrhodamine, sulforhodamine, thiorhodamine, cyanine, indocarbocyanine, oxacarbocyanine, thiacarbocyanine, merocyanine, cyanine 2, cyanine 3, cyanine 3.5, cyanine 5, cyanine 5.5, cyanine 7, oxadiazole derivatives, pyridyloxazole, nitrobenzoxadiazole, benzoxadiazole, pyren derivatives, cascade blue, oxazine derivatives, Nile red, Nile blue, cresyl violet, oxazine 170, acridine derivatives, proflavin, acridine orange, acridine yellow, arylmethine derivatives, auramine, crystal violet, malachite green, tetrapyrrole derivatives, porphin, phtalocyanine, bilirubin 1-dimethylaminonaphthyl-5-sulfonate, 1-anilino-8-naphthalene sulfonate, 2-p-touidinyl-6-naphthalene sulfonate, 3-phenyl-7-isocyanatocoumarin, N-(p-(2-benzoxazolyl)phenyl)maleimide, stilbenes, pyrenes, 6-FAM (Fluorescein), 6-FAM (NHS Ester), 5(6)-FAM, 5-FAM, Fluorescein dT, 5-TAMRA-cadavarine, 2-aminoacridone, HEX, JOE (NHS Ester), MAX, TET, ROX, and TAMRA.

In some embodiments, X comprises a fluorophore moiety. In some cases, the fluorophore of X is obtained from a compound library. In some cases, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

In some embodiments, the detectable labeling group is selected from the group comprising a member of a specific binding pair (e.g., biotin:streptavidin, antigen-antibody, nucleic acid:nucleic acid), a bead, a resin, a solid support, or a combination thereof. In some embodiments, the detectable labeling group is a biotin moiety, a streptavidin moiety, bead, resin, a solid support, or a combination thereof. In some embodiments, the detectable labeling group comprises biotin or a derivative thereof (e.g., desthiobiotin). In some embodiments, the detectable labeling group comprises a heavy isotope (i.e., ¹³C).

In some embodiments, X is a monovalent moiety comprising an alkyne group (i.e., a carbon-carbon triple bond). For example, in some embodiments, X comprises or consists of —C≡CH, -alkylene-C≡CH, —C(═O)-alkylene-C≡CH, or —C(═O)—NH-alkylene-C≡CH (e.g., C(═O)—NH—CH₂—C≡CH). In some embodiments, the alkylene group is a C₁-C₅ alkylene group. In some embodiments, the alkylene group is methylene. In some embodiments, X is a propargyl group, i.e., —CH₂—C≡CH.

In some embodiments, one of R₁ and R₂ is halo (e.g., chloro or fluoro) and the other of R₁ and R₂ is alkoxy, alkylthio or alkylamino. In some embodiments, one of R₁ and R₂ is alkylthio and the other of R₁ and R₂ is chloro or fluoro. In some embodiments, the compound is a compound shown in Scheme 8, below in Example 4, i.e., 6-(butylthio)-2-chloro-7-(prop-2-yn-1-yl)-7H-purine (Pa-1), 2-(butylthio)-6-chloro-7-(prop-2-yn-1-yl)-7H-purine (Pa-2), 2-(butylthio)-6-chloro-9-(prop-2-yn-1-yl)-9H-purine (Pa-3), 6-(butylthio)-2-chloro-9-(prop-2-yn-1-yl)-9H-purine (Pa-4), 6-(butylthio)-2-fluoro-7-(prop-2-yn-1-yl)-7H-purine (Pa-5), or 6-(butylthio)-2-fluoro-9-(prop-2-yn-1-yl)-9H-purine (Pa-6). In some embodiments, the compound is Pa-2, Pa-4, or Pa-6.

In some embodiments, the probe is selected from the group comprising 2,6-difluoro-7-(prop-2-yn-1-yl)-7H-purine, 2,6-difluoro-9-(prop-2-yn-1-yl)-9H-purine, and 6-chloro-2-fluoro-7-(prop-2-yn-1-yl)-7H-purine.

In some embodiments, the compound of Formula (III) is not one of the compounds selected from the group comprising 7-allyl-2,6-dichloro-7H-purine, 9-allyl-2,6-dichloro-9H-purine, 2,6-dichloro-7-benzyl-7H-purine, 2,6-dichloro-9-benzyl-9H-purine, 2,6-dichloro-9-(4-nitrobenzyl-9H-purine, and 2-(2,6-dichloro-9H-purin-9-yl)-5-(hydroxymethyl)tetrahydrofuran-3,4-diol.

In some embodiments, the N7-substituted regioisomer of the probe (i.e., the compound of Formula (Ib)) is provided with a purity of at least about 90% or more (e.g., about 90, 91, 92, 93, 94, 95, 96, 97, 98, or about 99% or more).

In some embodiments, e.g., as the presently disclosed probe compounds can be used to detect reactive amino acid residues in proteins in biological organisms, such as animals, the probe compound can be provided as a pharmaceutically acceptable salt or in a pharmaceutically acceptable carrier or formulation, such as a pharmaceutically acceptable carrier or formulation.

### IV. Ligands

Small molecules can serve as versatile ligands for perturbing the functions of proteins in biological systems. In some instances, a plurality of human proteins lack selective chemical ligands. In some cases, several classes of proteins are further considered as undruggable. Covalent purine-based ligands (also referred to herein as “fragments”) offer a strategy to expand the landscape of proteins amenable to targeting by small molecules. In some instances, covalent ligands combine features of recognition and reactivity, thereby providing for the targeting of sites on proteins that are difficult to address by reversible binding interactions alone.

In some embodiments, a ligand of the presently disclosed subject matter can compete with a probe compound described herein for binding with a reactive amino acid residue (e.g., a reactive cysteine residue). Often, the presently disclosed ligands are non-naturally occurring, and/or form non-naturally occurring products (modified proteins) after reaction with the nucleophilic group (e.g., the thiol group) of an amino acid residue (e.g., a cysteine residue).

In some embodiments, the ligand can modify one or more activity of the protein. For example, covalent attachment of a ligand to an enzyme can inhibit or activate an enzyme. In some embodiments, covalent attachment of a ligand to a protein can disrupt one or more protein-protein interactions of the modified protein. In some embodiments, covalent attachment of a ligand can disrupt protein-RNA interactions of the modified protein. In some embodiments, covalent attachment of a ligand can disrupt protein-DNA interactions of the modified protein. In some embodiments, covalent attachment of a ligand can disrupt protein-lipid interactions of the modified protein. In some embodiments, covalent attachment of a ligand can disrupt protein-metabolite interactions of the modified protein. In some embodiments, covalent attachment of a ligand can disrupt subcellular localization of the modified protein. In some embodiments, covalent attachment of a ligand can recruit an E3 ligase for targeted degradation of the modified protein. For instance, without being bount to any one theory, it is believed that covalent modification of a target protein with the probe can result in a protein-purine adduct that can be recognized by an E3 ligase, leading to binding, attachment of a polyubiquitin signal, and degradation of the target protein by the ubiquitin-proteosome system.

In some embodiments, the presently disclosed subject matter provides a purine-based compound that can form a covalent bond with a nucleophilic group of a side chain of a reactive amino acid residue (e.g., a reactive cysteine residue). In some embodiments, the presently disclosed subject matter provides a compound having a structure of Formula (III):

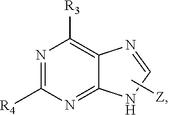

wherein Z is selected from the group comprising alkyl (e.g., C₁-C₆ alkyl), substituted alkyl, cycloalkyl (e.g., C₃-C₆ cycloalkyl), acyl (e.g., C₂-C₂₄ acyl or C₂-C₁₂ acyl), substituted acyl, aralkyl (e.g., benzyl, ethylphenyl, methylnaphthyl), substituted aralkyl (e.g., substituted benzyl), sulfonyl (i.e., —S(═O)₂—R₅), sulfonamide (i.e., —S(═O)₂—N(R₆)₂), and sulfonate (i.e., —S(═O)₂—O—R₇); R₃ and R₄ are independently selected from H, halo, alkyl (e.g., C₁-C₆ alkyl), alkoxy (e.g., C₁-C₆ alkoxy), alkylamino (e.g., C₁-C₆ alkylamino), alkylthio (C₁-C₆ alkylthio), aryloxy, arylamino, and arylthiol, subject to the proviso that at least one of R₃ and R₄ is halo, optionally chloro or fluoro; R₅ is heterocyclyl, substituted heterocyclyl, aryl or substituted aryl; each R₆ is selected from H, alkyl (e.g., C₁-C₆ alkyl), substituted alkyl (e.g., substituted C₁-C₆ alkyl), aralkyl, substituted aralkyl, aryl, and substituted aryl, or wherein the two R₆ together form an alkylene group (e.g., butylene or pentylene); and R₇ is selected from alkyl (e.g., C₁-C₆ alkyl), substituted alkyl, aralkyl, substituted aralkyl, aryl and substituted aryl. In some embodiments, Z is selected from the group comprising cycloalkyl, acyl, —S(═O)₂—R₅, —S(═O)₂—N(R₆)₂, —S(═O)₂—O—R₇, and

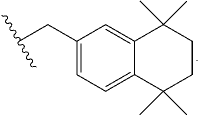 [SMILES: *Cc1ccc2c(c1)C(C)(C)CCC2(C)C]

In some embodiments, the compound of Formula (III) has a structure of Formula (IIIa):

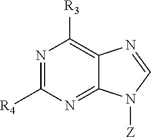

or a structure of Formula (IIIb):

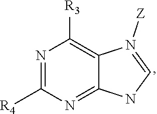

wherein the variables Z, R₃, R₄, R₅, R₆, and R₇ are as defined for the compound of Formula (III).

In some embodiments, R₃ is selected from chloro, fluoro, C₁-C₆ alkyl (e.g., methyl, ethyl, propyl, isopropyl, allyl, m-butyl, tert-butyl, pentyl, or hexyl), alkylthio, alkylamino, or aryloxy, optionally wherein the aryl group of the aryl oxy is substituted by one or more aryl group substituents (e.g., alkyl). In some embodiments, R₃ is selected from chloro, methyl, —SH—(CH₂)₃CH₃; —NH(CH₂)₃CH₃; and —O—(C₆H₄)CH₃.

In some embodiments, R₄ is chloro or fluoro.

In some embodiments, Z is selected from the group comprising C₂-C₁₂ acyl (e.g., acetyl, n-hexanoyl, or n-dodecanoyl); cycloalkyl (e.g., cyclohexyl), —S(═O)₂—R₅, —S(═O)₂—N(R₆)₂, —S(═O)₂—O—R₇, and

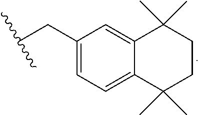 [SMILES: *Cc1ccc2c(c1)C(C)(C)CCC2(C)C]

In some embodiments, Z is —S(═O)₂—R₅, wherein R₅ is heterocyclyl (e.g., morpholine) or substituted phenyl. In some embodiments, the substituted phenyl is an alkoxy- or halo-substituted phenyl (e.g., 4-methoxyphenyl or 4-fluorophenyl).

In some embodiments, Z is —S(═O)₂—N(R₆)₂, wherein each R₆ is selected from alkyl and aralkyl. In some embodiments, at least one R₆ is alkyl, e.g., methyl, ethyl, propyl, butyl, pentyl or hexyl. In some embodiments, both R₆ are alkyl. In some embodiments, both R₆ are ethyl. In some embodiments, one R₆ is aralkyl, e.g., benzyl.

In some embodiments, Z is —S(═O)₂—O—R₇, wherein R₇ is alkyl or aralkyl. In some embodiments, R₇ is alkyl, e.g., methyl, ethyl, propyl, butyl, pentyl, or hexyl. In some embodiments, R₇ is methyl. In some embodiments, R₇ is benzyl.

In some embodiments, Z is selected from the group comprising:

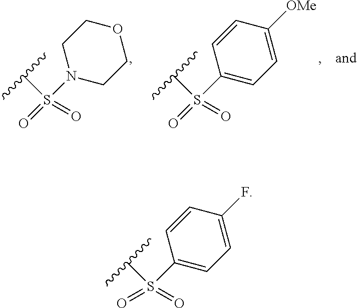 [SMILES: *S(=O)(=O)N1CCOCC1 ; *S(=O)(=O)c1ccc(OC)cc1 ; *S(=O)(=O)c1ccc(F)cc1]

In some embodiments, the compound of Formula (III) is selected from the group comprising: 4-((2,6-dichloro-7H-purin-7-yl)sulfonyl)morpholine (Pi-7), 4-((2,6-dichloro-9H-purin-9-yl)sulfonyl)morpholine (Pi-8), 2,6-dichloro-7-((4-fluorophenyl)sulfonyl)-7H-purine (Pi-13), 2,6-dichloro-9-((4-fluorophenyl)sulfonyl)-9H-purine (Pi-14), 2,6-dichloro-7-((4-methoxyphenyl)sulfonyl)-7H-purine (Pi-15), 2,6-dichloro-9-((4-methoxyphenyl)sulfonyl)-9H-purine (Pi-16), 2,6-dichloro-7-((5,5,8,8-tetramethyl-5,6,7,8-tetrahydronaphthalen-2-yl)methyl)-7H-purine (Pi-11), 2,6-dichloro-9-((5,5,8,8-tetramethyl-5,6,7,8-tetrahydronaphthalen-2-yl)methyl)-9H-purine (Pi-12), 1-(2,6-dichloro-9H-purin-9-yl)dodecan-1-one, 1-(2,6-dichloro-7H-purin-7-yl)hexan-1-one, 1-(2,6-dichloro-9H-purin-9-yl)hexan-1-one, 1-(6-chloro-2-fluoro-7H-purin-7-yl)hexan-1-one, 1-(6-chloro-2-fluoro-9H-purin-9-yl)hexan-1-one, 1-(2-chloro-6-methyl-9H-purin-9-yl)hexan-1-one, 2-chloro-9-cyclohexyl-6-methyl-9H-purine, 9-cyclohexyl-2-fluoro-6-methyl-9H-purine, 1-(6-(butylthio)-2-fluoro-9H-purin-9-yl)ethan-1-one, 1-(6-(butylthio)-2-chloro-9H-purin-9-yl)ethan-1-one (AHL20-001), 1-(6-(butylthio)-2-chloro-7H-purin-7-yl)ethan-1-one, 1-(6-(butylthio)-2-fluoro-7H-purin-7-yl)ethan-1-one, 1-(2-chloro-6-(p-tolyloxy)-9H-purin-9-yl)ethan-1-one, 1-(2-fluoro-6-(p-tolyloxy)-9H-purin-9-yl)ethan-1-one, 1-(2-chloro-6-(p-tolyloxy)-7H-purin-7-yl)ethan-1-one, 1-(2-fluoro-6-(p-tolyloxy)-7H-purin-7-yl)ethan-1-one, 1-(6-(butylamino)-2-chloro-7H-purin-7-yl)ethan-1-one, 1-(6-(butylamino)-2-fluoro-7H-purin-7-yl)ethan-1-one, 1-(6-(butylamino)-2-chloro-9H-purin-9-yl)ethan-1-one, and 1-(6-(butylamino)-2-fluoro-9H-purin-9-yl)ethan-1-one.

In some embodiments, the compound is selected from the group comprising 2,6-dichloro-N,N-diethyl-7H-purine-7-sulfonamide, 2,6-dichloro-N,N-diethyl-9H-purine-9-sulfonamide, N-benzyl-2,6-dichloro-N-methyl-9H-purine-9-sulfonamide, N-benzyl-2,6-dichloro-N-methyl-7H-purine-7-sulfonamide, benzyl 2,6-dichloro-7H-purine-7-sulfonate, benzyl 2,6-dichloro-9H-purine-9-sulfonate, methyl 2,6-dichloro-9H-dichloro-9-purine-9-sulfonate, and methyl 2,6-dichloro-7H-purine-7-sulfonate.

In some embodiments, the compound is 2,6-dichloro-7-(4-nitrobenzyl)-7H-purine.

In some embodiments, the compound is a N7-substituted regioisomer and has a purity of at least about 90% or more (e.g., about 90, 91, 92, 93, 94, 95, 96, 97, 98 or about 99% or more).

The presently disclosed subject matter encompasses the preparation and use of pharmaceutical compositions comprising a ligand compound as described herein useful for treatment of diseases and disorders as would be apparent upon review of the instant disclosure as an active ingredient. Such a pharmaceutical composition can comprise, consist essentially of, or consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter can comprise at least one active ingredient, one or more acceptable carriers, and optionally other active ingredients or therapeutic agents.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter or pharmaceutical compositions comprising these compositions can be administered so that the compositions may have a physiological effect. Administration can occur enterally or parenterally; for example, orally, rectally, intracisternally, intravaginally, intraperitoneally, locally (e.g., with powders, ointments or drops), or as a buccal or nasal spray or aerosol. Parenteral administration is an approach. Particular parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, and direct application to the target area, e.g., intratumoral injection, for example by a catheter or other placement device.

Where the administration of the composition is by injection or direct application, the injection or direct application can be in a single dose or in multiple doses. Where the administration of the compound is by infusion, the infusion can be a single sustained dose over a prolonged period of time or multiple infusions.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially and/or socially relevant mammals such as cattle, pigs, horses, sheep, cats, and dogs, birds including commercially and/or socially relevant birds such as chickens, ducks, geese, parrots, and turkeys.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient.

In addition to the active ingredient, a pharmaceutical composition of the presently disclosed subject matter can further comprise one or more additional pharmaceutically active agents.

Controlled- or sustained-release formulations of a pharmaceutical composition of the presently disclosed subject matter can be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro (1990) Remington's Pharmaceutical Sciences, 18th ed., Mack Pub. Co., Easton, Pa., United States of America and/or Gennaro (ed.) (2003) Remington: The Science and Practice of Pharmacy, 20th edition Lippincott, Williams & Wilkins, Philadelphia, Pa., United States of America, each of which is incorporated herein by reference.

The compositions may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Other approaches include but are not limited to nanosizing the composition comprising a ligand compound as described herein to be delivered as a nanoparticle intravenously, intraperitoneal injection, or implanted beads with time release of a ligand compound as described herein.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a cell or a tissue of a subject. In some embodiments, this kit comprises a (in some embodiments sterile) solvent suitable for dissolving or suspending the composition of the presently disclosed subject matter prior to administering the compound to the subject and/or a device suitable for administering the composition such as a syringe, injector, or the like or other device as would be apparent to one of ordinary skill in the art upon a review of the instant disclosure.

As used herein, an “instructional material” includes a publication, a recording, a diagram, or any other medium of expression which can be used to communicate the usefulness of the composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter can, for example, be affixed to a container which contains a composition of the presently disclosed subject matter or be shipped together with a container which contains the composition. Alternatively, the instructional material can be shipped separately from the container with the intention that the instructional material and the composition be used cooperatively by the recipient.

### V. Synthesis

The probes and ligands of the presently disclosed subject matter can be prepared using organic group transformations known in the art of organic synthesis and as further described in the Examples below.

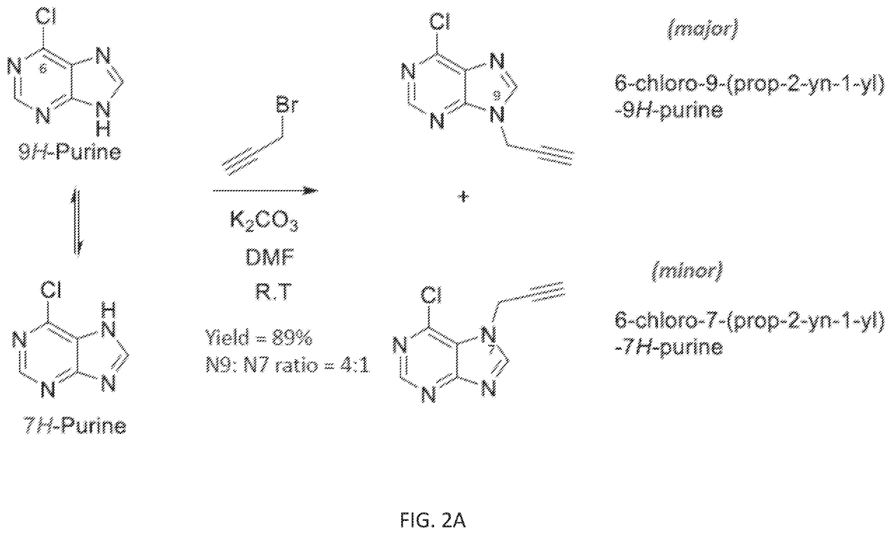

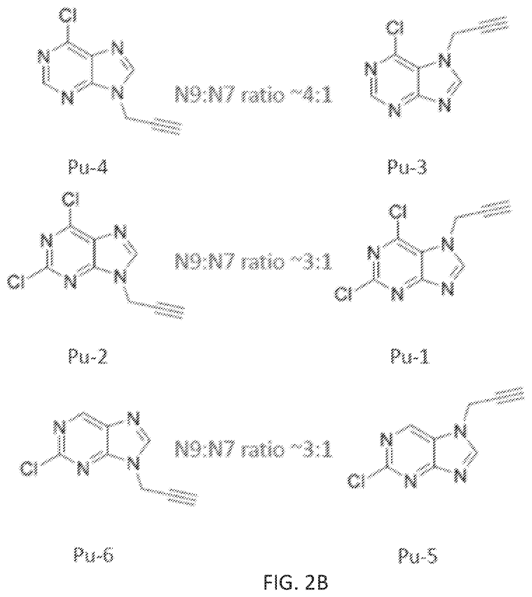

In some embodiments, the presently disclosed purine-based probes and ligands can be prepared by contacting a halo- or di-halo purine with a reagent that can react with one of the amines of the imidazole ring. For example, the halo- or di-halo-substituted purine-based probe or ligand can be prepared by contacting a halo- or di-halo-substituted purine with a halide (e.g., propargyl bromide or another alkyl halide, a benzyl bromide or another aralkyl halide, etc) in the presence of a base (e.g., potassium carbonate or sodium carbonate). See FIG. 2A. The reactions can be performed in a suitable solvent, e.g., an aprotic organic solvent, such as dimethylformamide (DMF) or tetrahydrofuran (THF). These reactions can result in two different regioisomers, the N9 isomer and the more sterically hindered N7 isomer. Typically, the N9 isomer is the major product. See FIG. 2B. However, reports in the literature have described that N-alkylation can occur at the more sterically hindered nitrogen atom of various 1,3-azoles if an organomagnesium reagent is used as a base.¹⁷ Thus, if desired, the N7 isomer of the presently disclosed probes and ligands can be made as the major isomer by addition of, for example, three equivalents of methyl magnesium chloride or another organomagnesium reagent.

Adducts of the halo or dihalo purine probes or ligands, e.g., where the 6-halo substituent is replaced by an alkoxy, aryloxy, alkylthio, arylthiol, alkylamino or arylamino group can be prepared by reacting the halo- or di-halo purines with a thiol, amine, alcohol or phenol in the presence of a hindered/non-nucleophilic base, such as Hunig's base (i.e., N,N-diisopropylethylamine) or triethylamine.

Acylated purine ligands can be prepared by contacting a halo-substituted purine or an alkoxy, alkylthio, alkylamino, aryloxy, arylthiol, or arylamino adduct thereof with an anhydride or acid chloride. Sulfonated purine ligands can be prepared by contacting a halo-substituted purine or an alkoxy, alkylthio, alkylamino, aryloxy, arylthiol, or arylamino adduct thereof with a suitable activated sulfonyl compound, such as a sulfonyl chloride.

Scheme 2, below, shows the compounds prepared according to the methods described above using the following commercially reagents used without further purification: benzyl bromide, allyl bromide, 4-nitrophenyl bromide, 6-(Bromomethyl)-1,1,4,4-tetramethyl-1,2,3,4-tetrahydronaphthalene, morpholine-4-sulfonyl chloride, 4-methoxyphenylsulfonyl chloride, 4-fluorophenylsulfonyl chloride 1-butanethiol and acetic anhydride.

Scheme 3, below, shows a synthetic route to sulfonamide- and sulfonate-substituted purine-based ligands of the presently disclosed subject matter. Exemplary sulfonamide- and sulfonate-substituted purine-based ligands can be prepared from a halo-substituted purine using commercially available sulfamoyl halides and esters of halosulfuric acids (e.g., esters of chlorosulfuric acid or sulfurochloridates), such as sulfamoyl chloride, dimethylsulfamoyl chloride, diethyl sulfamoyl chloride, ethyl(phenyl)sulfamoyl chloride, methyl(phenyl)sulfamoyl chloride, diphenylsulfamoyl chloride, benzyl(methyl)sulfamoyl chloride, phenyl sulfurochloridate, isopentyl sulfurochloridate, methyl sulfurochloridate, and 4-methyoxybenzyl sulfurochloridate.

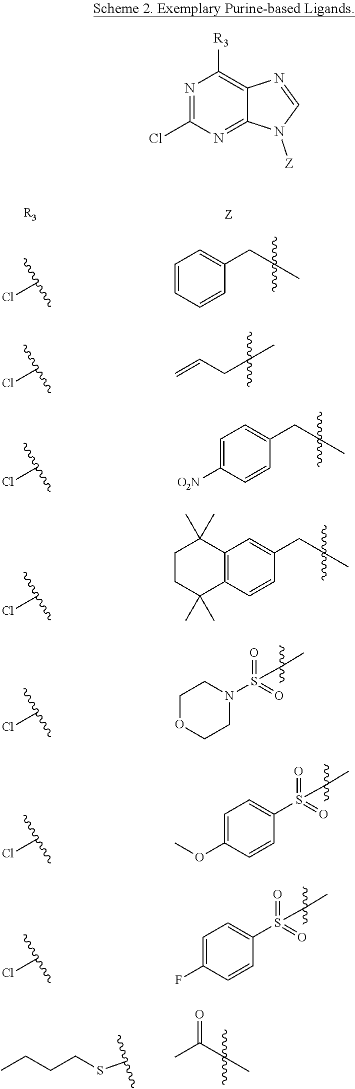

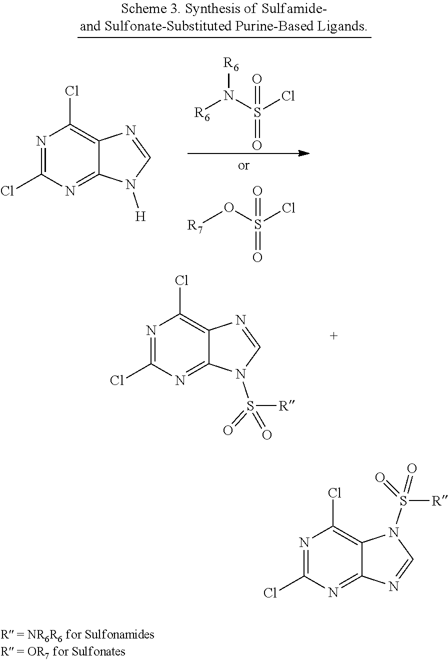

### VI. Modified Proteins

In some embodiments, the presently disclosed subject matter provides a modified cysteine-containing protein. The modified protein can be a protein comprising the adduct formed between a cysteine thiol side chain group and a probe or ligand of the presently disclosed subject matter. The modified protein can have a different biological activity than the unmodified protein.

In some embodiments, the presently disclosed subject matter provides a modified cysteine-containing protein comprising a modified cysteine residue wherein the modified cysteine residue is formed by the reaction of a cysteine residue of a non-naturally occurring purine-based compound (e.g., a halo-substituted purine). In some embodiments, the non-naturally occurring purine-based compound is a compound having a structure of Formula (I):

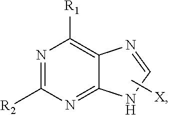

or a compound having a structure of Formula (III′):

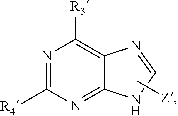

wherein X is a monovalent moiety comprising an alkyne moiety, a fluorophore moiety, a detectable labeling group, or a combination thereof; Z′ is selected from the group comprising alkyl (e.g., C₁-C₆ alkyl), substituted alkyl, cycloalkyl (e.g., C₃-C₆ cycloalkyl), heterocycloalkyl, acyl (e.g., C₂-C₂₄ acyl or C₂-C₁₂ acyl), substituted acyl, aralkyl (e.g., benzyl, ethylbenzyl, methylnaphthyl), substituted aralkyl (e.g., substituted benzyl), —S(═O)₂—R₅′, —S(═O)₂—N(R₆)₂, and —S(═O)₂—O—R₇; R₁ and R₂ are independently selected from the group comprising H, halo, hydroxyl, thiol, amino, alkyl (e.g., C₁-C₆ alkyl), alkoxy (e.g., C₁-C₆ alkoxy), alkylamino (e.g., C₁-C₆ alkylamino), alkylthio (e.g., C₁-C₆ alkylthio), aryloxy, arylamino, and arylthio, subject to the proviso that at least one of R₁ and R₂ is halo; R₃′ and R₄′ are independently selected from H, halo, alkyl (e.g., C₁-C₆ alkyl), alkylamino (e.g., C₁-C₆ alkylamino), alkylthio (e.g., C₁-C₆ alkylthio), alkoxy (e.g., C₁-C₆ alkoxy), aryloxy, arylamino, and arylthio, subject to the proviso that at least one of R₃′ and R₄′ is halo; R₅′ is heterocyclyl, substituted heterocyclyl, aryl or substituted aryl; each R₆ is selected from H, alkyl (e.g., C₁-C₆ alkyl), substituted alkyl, aralkyl, substituted aralkyl, aryl, and substituted aryl, or wherein the two R₆ together form an alkylene group; and R₇ is selected from alkyl (e.g., C₁-C₆ alkyl), substituted alkyl, aralkyl, substituted aralkyl, aryl and substituted aryl.

In some embodiments, the modified cysteine-containing protein comprises at least one modified cysteine residue comprising a structure of Formula (II-i):

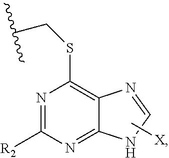

a structure of Formula (II-ii):

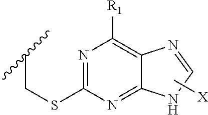

a structure of Formula (IV′-i):

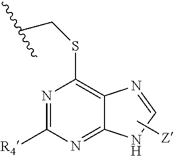

or a structure of Formula (IV′-ii):

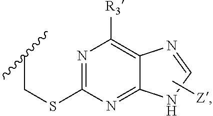

wherein X, Z′, R₅′, R₆, and R₇ are as defined for the compounds of Formula (I) or Formula (III′) and wherein R₁ is selected from the group consisting of H, halo, hydroxyl, thiol, amino, alkyl, alkoxy, alkylamino, alkylthio, aryloxy, arylamino, and arylthio; R₂ is selected from the group consisting of H, halo, hydroxyl, thiol, amino, alkyl, alkoxy, alkylamino, alkylthio, aryloxy, arylamino, and arylthio; R₃′ selected is from H, halo, alkyl, alkylamino, alkylthio, alkoxy, aryloxy, arylamino, and arylthiol; and R₄′ is selected from H, halo, alkyl, alkylamino, alkylthio, alkoxy, aryloxy, arylamino, and arylthiol. In some embodiments, X or Z′ is attached to the N7 nitrogen atom. In some embodiments, X or Z′ is attached to the N9 nitrogen atom.

In some embodiments, X comprises a fluorophore or a detectable labeling group, such as a fluorophore or detectable labeling group as defined hereinabove. In some embodiments, X is a monovalent moiety comprising an alkyne group. For example, in some embodiments, X comprises or consists of —C≡CH, -alkylene-C≡CH, —C(═O)-alkylene-C≡CH, or —C(═O)—NH-alkylene-C≡CH (e.g., C(═O)—NH—CH₂—C≡CH). In some embodiments, the alkylene group is a C₁-C₅ alkylene group. In some embodiments, the alkylene group is methylene. In some embodiments, X is a propargyl group, i.e., —CH₂—C≡CH.

In some embodiments, Z′ is selected from C₁-C₆ alkyl (e.g., allyl), a sugar residue, benzyl or substituted benzyl (e.g., 4-nitrobenzyl). In some embodiments, Z′ is selected from the group comprising acyl, cycloalkyl (e.g., cyclohexyl), —S(═O)₂—R₅′, —S(═O)₂—N(R₆)₂, —S(═O)₂—O—R₇ and

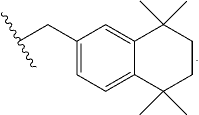 [SMILES: *Cc1ccc2c(c1)C(C)(C)CCC2(C)C]

In some embodiments, Z′ is —S(═O)₂—R₅′, wherein R₅′ is heterocyclyl (e.g., morpholine) or substituted phenyl. In some embodiments, the substituted phenyl is an alkoxy- or halo-substituted phenyl (e.g., 4-methoxyphenyl or 4-fluorophenyl). In some embodiments, Z′ is —S(═O)₂—N(R₆)₂, wherein each R₆ is selected from alkyl and aralkyl. In some embodiments, at least one R₆ is alkyl, e.g., methyl, ethyl, propyl, butyl, pentyl or hexyl. In some embodiments, both R₆ are alkyl. In some embodiments, both R₆ are ethyl. In some embodiments, one R₆ is aralkyl, e.g., benzyl. In some embodiments, Z′ is —S(═O)₂—O—R₇, wherein R₇ is alkyl or aralkyl. In some embodiments, R₇ is alkyl, e.g., methyl, ethyl, propyl, butyl, pentyl, or hexyl. In some embodiments, R₇ is methyl. In some embodiments, R₇ is benzyl.

In some embodiments, Z′ is selected from the group comprising:

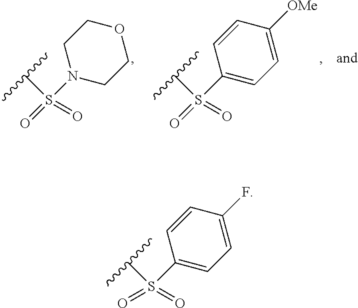 [SMILES: *S(=O)(=O)N1CCOCC1 ; *S(=O)(=O)c1ccc(OC)cc1 ; *S(=O)(=O)c1ccc(F)cc1]

In some embodiments, R₁, R₂, R₃′ or R₄′ is selected from chloro, fluoro, C₁-C₆ alkyl (e.g., methyl, ethyl, propyl, isopropyl, allyl, m-butyl, tert-butyl, pentyl, or hexyl), alkylthio, alkylamino, or aryloxy, optionally wherein the aryl group of the aryloxy is substituted by one or more aryl group substituents. In some embodiments, the R₁, R₂, R₃′ or R₄′ is selected from chloro, methyl, —SH—(CH₂)₃CH₃; —NH(CH₂)₃CH₃; and —O—(C₆H₄)CH₃.

In some embodiments, the modified cysteine-containing protein is a cysteine-containing protein listed in Table 3 or Table 4, below, e.g., modified at one of the cysteine residues noted in the tables. In some embodiments, the modified cysteine-containing protein is modified in a domain selected from the group comprising ADF-H domain, calponin-homology (CH) domain, WWE domain, translation-type guanine nucleotide binding (G) domain, elongation factor 1 (EF-1) gamma C-terminal domain, protein kinase domain, Bin3-type S-adenosyl-L-methionine domain, CXC domain, PITH domain, WHEP-TRS domain, mRNA (guanine-N(7)-methyl transferase domain, CoA carboxytransferase domain, and thermonuclease domain.

In some embodiments, the modified cysteine-containing protein is selenocysteine elongation factor (eEF-Sec) modified at cysteine 442, macrophage migration inhibitory factor modified at cysteine 81; or serine/threonine protein kinase 38-like modified at cysteine 235.

### VII. Methods of Modulating Protein Activity

In some embodiments, presently disclosed subject matter provides a method of modulating the activity of a protein comprising a reactive amino acid residue by contacting the protein with a halo-substituted purine compound, such as a probe or ligand of the presently disclosed subject matter. In some embodiments, the presently disclosed subject matter provides a method of modulating the activity of a protein comprising a reactive cysteine residue. In some embodiments, the protein with the reactive amino acid residue is an enzyme and modulating the activity of the protein comprises inhibiting or activating the enzyme. In some embodiments, modulating the activity of a protein comprises enhancing or reducing the ability of the protein to interact with other compounds, such as other proteins. Thus, in some embodiments, the modulation results in reducing the protein-protein interactions of the protein comprising the reactive amino acid.

In some embodiments, the presently disclosed subject matter provides a method of modulating the activity of a protein comprising a reactive cysteine residue, wherein the method comprising contacting a protein comprising a reactive cysteine residue with a compound having a structure of Formula (III′):

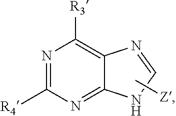

wherein Z′ is selected from the group comprising alkyl (e.g., C₁-C₆ alkyl), substituted alkyl, cycloalkyl (e.g., C₃-C₆ cycloalkyl), heterocycloalkyl, acyl (e.g., C₂-C₂₄ acyl or C₂-C₁₂ acyl), substituted acyl, aralkyl (e.g., benzyl), substituted aralkyl (e.g., substituted benzyl, ethylbenzyl, methylnaphthyl), —S(═O)₂—R₅′, —S(═O)₂—N(R₆)₂, and —S(═O)₂—O—R₇; R₃′ and R₄′ are independently selected from H, halo, alkyl (e.g., C₁-C₆ alkyl), alkylamino (e.g., C₁-C₆ alkylamino), alkylthio (e.g., C₁-C₆ alkylthio), alkoxy (e.g., C₁-C₆ alkoxy), aryloxy, arylamino, and arylthio, subject to the proviso that at least one of R₃′ and R₄′ is halo (e.g., chloro or fluoro); R₅′ is heterocyclyl, substituted heterocyclyl, aryl or substituted aryl; each R₆ is selected from H, alkyl (e.g., C₁-C₆ alkyl), substituted alkyl, aralkyl, substituted aralkyl, aryl, and substituted aryl, or wherein the two R₆ together form an alkylene group; and R₇ is selected from alkyl (e.g., C₁-C₆ alkyl), substituted alkyl, aralkyl, substituted aralkyl, aryl and substituted aryl.

In some embodiments, Z′ is substituted on the N7 or N9 atom and the compound having a structure of Formula (III′) is a compound having a structure of Formula (IIIa′):

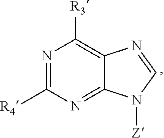

or a structure of Formula (IIIb′):

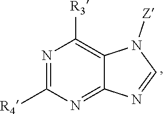

wherein Z′, R₃′, and R₄′ are as defined for Formula (III′).

In some embodiments, R₃′ is halo, alkyl, alkyoxy, alkylthio, alkylamino, or aryloxy. In some embodiments, R₃′ is selected from chloro, fluoro, methyl, n-butylthio, n-butylamino, and —O—(C₆H₄)—OMe. In some embodiments, R₄′ is halo. In some embodiments, R₄′ is fluoro or chloro. In some embodiments, both R₃′ and R₄′ are halo. In some embodiments, R₃′ and R₄′ are each independently selected from chloro and fluoro. In some embodiments, R₃′ and R₄′ are both chloro.

In some embodiments, Z′ is selected from C₁-C₆ alkyl (e.g., allyl), a sugar residue, benzyl or substituted benzyl (e.g., 4-nitrobenzyl). In some embodiments, Z′ is selected from the group comprising acyl, cycloalkyl, —S(═O)₂—R₅′, —S(═O)₂—N(R₆)₂, —S(═O)₂—O—R₇ and

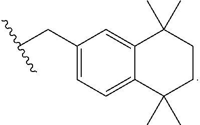 [SMILES: *Cc1ccc2c(c1)C(C)(C)CCC2(C)C]

In some embodiments, Z′ is selected from —CH₂—CH═CH₂, C₂-C₁₂ acyl (e.g., acetyl, hexanoyl, or dodecanoyl), cyclohexyl, benzyl, —CH₂—(C₆H₄)—NO₂, —S(═O)₂—R₅′, and

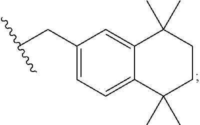

wherein R₅′ is selected from heterocyclyl and substituted aryl (e.g., wherein R₅′ is selected from morpholinyl, 4-halophenyl, and 4-alkoxyphenyl).

In some embodiments, Z′ is —S(═O)₂—R₅′, wherein R₅′ is heterocyclyl (e.g., morpholine) or substituted phenyl. In some embodiments, the substituted phenyl is an alkoxy- or halo-substituted phenyl (e.g., 4-methoxyphenyl or 4-fluorophenyl). In some embodiments, R₅′ is selected from morpholine and 4-substituted phenyl. In some embodiments, R₅′ is selected from morpholine, 4-halophenyl, and 4-alkoxyphenyl. In some embodiments, R₅′ is selected from morpholine, 4-fluorophenyl, and 4-methyoxyphenyl. In some embodiments, Z′ is —S(═O)₂—N(R₆)₂, wherein each R₆ is selected from alkyl and aralkyl. In some embodiments, at least one R₆ is alkyl, e.g., methyl, ethyl, propyl, butyl, pentyl or hexyl. In some embodiments, both R₆ are alkyl. In some embodiments, both R₆ are ethyl. In some embodiments, one R₆ is aralkyl, e.g., benzyl. In some embodiments, Z′ is —S(═O)₂—O—R₇, wherein R₇ is alkyl or aralkyl. In some embodiments, R₇ is alkyl, e.g., methyl, ethyl, propyl, butyl, pentyl, or hexyl. In some embodiments, R₇ is methyl. In some embodiments, R₇ is benzyl.

In some embodiments, Z′ is selected from the group comprising:

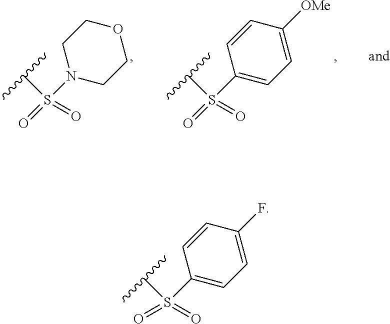 [SMILES: *S(=O)(=O)N1CCOCC1 ; *S(=O)(=O)c1ccc(OC)cc1 ; *S(=O)(=O)c1ccc(F)cc1]

In some embodiments, the compound of Formula (III′) is selected from the group comprising 4-((2,6-dichloro-7H-purin-7-yl)sulfonyl)morpholine, 4-((2,6-dichloro-9H-purin-9-yl)sulfonyl)morpholine, 2,6-dichloro-7-((4-fluorophenyl)sulfonyl)-7H-purine, 2,6-dichloro-9-((4-fluorophenyl)sulfonyl)-9H-purine, 2,6-dichloro-7-((4-methoxyphenyl)sulfonyl)-7H-purine, 2,6-dichloro-9-((4-methoxyphenyl)sulfonyl)-9H-purine, 2,6-dichloro-7-((5,5,8,8-tetramethyl-5,6,7,8-tetrahydronaphthalen-2-yl)methyl)-7H-purine, 2,6-dichloro-9-((5,5,8,8-tetramethyl-5,6,7,8-tetrahydronaphthalen-2-yl)methyl)-9H-purine, 2,6-dichloro-7-(4-nitrobenzyl)-7H-purine, 1-(2,6-dichloro-9H-purin-9-yl)dodecan-1-one, 1-(2,6-dichloro-7H-purin-7-yl)hexan-1-one, 1-(2,6-dichloro-9H-purin-9-yl)hexan-1-one, 1-(6-chloro-2-fluoro-7H-purin-7-yl)hexan-1-one, 1-(6-chloro-2-fluoro-9H-purin-9-yl)hexan-1-one, 1-(2-chloro-6-methyl-9H-purin-9-yl)hexan-1-one, 2-chloro-9-cyclohexyl-6-methyl-9H-purine, 9-cyclohexyl-2-fluoro-6-methyl-9H-purine, 1-(6-(butylthio)-2-fluoro-9H-purin-9-yl)ethan-1-one, 1-(6-(butylthio)-2-chloro-9H-purin-9-yl)ethan-1-one, 1-(6-(butylthio)-2-chloro-7H-purin-7-yl)ethan-1-one, 1-(6-(butylthio)-2-fluoro-7H-purin-7-yl)ethan-1-one, 1-(2-chloro-6-(p-tolyloxy)-9H-purin-9-yl)ethan-1-one, 1-(2-fluoro-6-(p-tolyloxy)-9H-purin-9-yl)ethan-1-one, 1-(2-chloro-6-(p-tolyloxy)-7H-purin-7-yl)ethan-1-one, 1-(2-fluoro-6-(p-tolyloxy)-7H-purin-7-yl)ethan-1-one, 1-(6-(butylamino)-2-chloro-7H-purin-7-yl)ethan-1-one, 1-(6-(butylamino)-2-fluoro-7H-purin-7-yl)ethan-1-one, 7-allyl-2,6-dichloro-7H-purine, 9-allyl-2,6-dichloro-9H-purine, 2,6-dichloro-7-benzyl-7H-purine, 2,6-dichloro-9-benzyl-9H-purine, 2,6-dichloro-9-(4-nitrobenzyl-9H-purine, 2-(2,6-dichloro-9H-purin-9-yl)-5-(hydroxymethyl)tetrahydrofuran-3,4-diol, 1-(6-(butylamino)-2-chloro-9H-purin-9-yl)ethan-1-one, 1-(6-(butylamino)-2-fluoro-9H-purin-9-yl)ethan-1-one, 2,6-dichloro-N,N-diethyl-7H-purine-7-sulfonamide, 2,6-dichloro-N,N-diethyl-9H-purine-9-sulfonamide, N-benzyl-2,6-dichloro-N-methyl-9H-purine-9-sulfonamide, N-benzyl-2,6-dichloro-N-methyl-7H-purine-7H-sulfonamide, benzyl 2,6-dichloro-7H-purine-7-sulfonate, benzyl 2,6-dichloro-9H-purine-9-sulfonate, methyl 2,6-dichloro-9H-purine-9-sulfonate, and methyl 2,6-dichloro-7H-purine-7-sulfonate.

In some embodiments, the compound of Formula (III′) is not one of the compounds selected from the group comprising 7-allyl-2,6-dichloro-7H-purine, 9-allyl-2,6-dichloro-9H-purine, 2,6-dichloro-7-benzyl-7H-purine, 2,6-dichloro-9-benzyl-9H-purine, 2,6-dichloro-9-(4-nitrobenzyl-9H-purine, and 2-(2,6-dichloro-9H-purin-9-yl)-5-(hydroxymethyl)tetrahydrofuran-3,4-diol.

In some embodiments, the compound of Formula (III′) is a compound of Formula (IIIb′) and has a purity of at least about 90% (e.g., at least about 90, 91, 92, 93, 94, 95, 96, 97, 98, or about 99% or more), e.g., by HPLC. Thus, in some embodiments, an N7-substituted purine-based compound is provided substantially free of the N9-substituted regioisomer.

In some embodiments, contacting the protein comprising a reactive cysteine residue with the compound of Formula (III′) provides a modified cysteine-containing protein comprising a structure of one of Formulas (IV′-i) and (IV′-ii) described hereinabove.

In some embodiments, modulating an activity of a protein comprising a reactive cysteine residue comprises inhibiting (partially or substantially completely) an activity (e.g., an enzymatic activity) of the protein comprising a reactive cysteine residue. In some embodiments, modulating an activity of a protein comprising a reactive cysteine residue comprises activating an activity (e.g., an enzymatic activity) of the protein comprising a reactive cysteine residue. In some embodiments, modulating the activity of a protein comprising a reactive cysteine residue comprises inhibiting, blocking (partially or substantially completely) or disrupting a protein-protein interaction, a protein-RNA interaction, a protein-DNA interaction, a protein-lipid interaction, and/or a protein-metabolite interaction of the protein comprising a reactive cysteine residue. In some embodiments, modulating an activity of a protein comprising a reactive cysteine residue comprises inhibiting or disrupting subcellular localization of the protein comprising a reactive cysteine residue. In some embodiments, modulating an activity of a protein comprising a reactive cysteine residue comprises triggering recruitment of an E3 ligase for targeted degradation of the protein comprising a reactive cysteine residue.

### VIII. Cells, Analytical Techniques and Instrumentation

In some embodiments, one or more of the methods disclosed herein comprise a sample (e.g., a cell sample, cell lysate sample or a biological organism). In some embodiments, the sample for use with the methods described herein is obtained from cells of an animal. In some instances, the animal cell includes a cell from a marine invertebrate, fish, insects, amphibian, reptile, or mammal. In some instances, the mammalian cell is a primate, ape, equine, bovine, porcine, canine, feline, or rodent. In some instances, the mammal is a primate, ape, dog, cat, rabbit, ferret, or the like. In some cases, the rodent is a mouse, rat, hamster, gerbil, hamster, chinchilla, or guinea pig. In some embodiments, the bird cell is from a canary, parakeet or parrots. In some embodiments, the reptile cell is from a turtles, lizard or snake. In some cases, the fish cell is from a tropical fish. In some cases, the fish cell is from a zebrafish (e.g. Danino rerio). In some cases, the worm cell is from a nematode (e.g. C. elegans). In some cases, the amphibian cell is from a frog. In some embodiments, the arthropod cell is from a tarantula or hermit crab.

In some embodiments, the sample for use with the methods described herein is obtained from a mammalian cell. In some instances, the mammalian cell is an epithelial cell, connective tissue cell, hormone secreting cell, a nerve cell, a skeletal muscle cell, a blood cell, or an immune system cell. Exemplary mammalian cell lines include, but are not limited to, 293A cells, 293FT cells, 293F cells, 293H cells, HEK 293 cells, CHO DG44 cells, CHO-S cells, CHO-K1 cells, and PC12 cells.

In some embodiments, the sample for use with the methods described herein is obtained from cells of a tumor cell line. In some instances, the sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, and telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CIVIL), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the sample for use with the methods described herein is obtained from a tumor cell line. Exemplary tumor cell lines include, but are not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, the sample for use in the methods is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the sample is a tissue sample, such as a sample obtained from a biopsy or a tumor tissue sample. In some embodiments, the sample is a blood serum sample. In some embodiments, the sample is a blood cell sample containing one or more peripheral blood mononuclear cells (PBMCs). In some embodiments, the sample contains one or more circulating tumor cells (CTCs). In some embodiments, the sample contains one or more disseminated tumor cells (DTC, e.g., in a bone marrow aspirate sample).

In some embodiments, the samples are obtained from the individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, will be examined under a microscope. In some embodiments, the sample is a biological organism. In some embodiments, the biological organism is a rodent, e.g., a mouse or a rat. In some embodiments, the biological organism is a primate, e.g., a monkey. In some embodiments, the biological organism is a bacteria or a fungi.

### IX. Sample Preparation and Analysis

In some embodiments, the sample (e.g., cell sample, cell lysate sample, or comprising isolated proteins) is a sample solution. In some instances, the sample solution comprises a solution such as a buffer (e.g. phosphate buffered saline) or a media. In some embodiments, the media is an isotopically labeled media. In some instances, the sample solution is a cell solution.

In some embodiments, the sample (e.g., cell sample, cell lysate sample, or comprising isolated proteins) is incubated with one or more compound probes for analysis of protein-probe interactions. In some instances, the sample (e.g., cell sample, cell lysate sample, or comprising isolated proteins) is further incubated in the presence of an additional compound probe prior to addition of the one or more probes. In other instances, the sample (e.g., cell sample, cell lysate sample, or comprising isolated proteins) is further incubated with a non-probe small molecule ligand, in which the non-probe small molecule ligand does not contain a photoreactive moiety and/or an alkyne group. In such instances, the sample is incubated with a probe and non-probe small molecule ligand for competitive protein profiling analysis.

In some cases, the sample is compared with a control. In some cases, a difference is observed between a set of probe protein interactions between the sample and the control. In some instances, the difference correlates to the interaction between the small molecule fragment and the proteins.

In some embodiments, one or more methods are utilized for labeling a sample (e.g. cell sample, cell lysate sample, or comprising isolated proteins) for analysis of probe protein interactions. In some instances, a method comprises labeling the sample (e.g. cell sample, cell lysate sample, or comprising isolated proteins) with an enriched media. In some cases, the sample (e.g. cell sample, cell lysate sample, or comprising isolated proteins) is labeled with isotope-labeled amino acids, such as ¹³C or ¹⁵N-labeled amino acids. In some cases, the labeled sample is further compared with a non-labeled sample to detect differences in probe protein interactions between the two samples. In some instances, this difference is a difference of a target protein and its interaction with a small molecule ligand in the labeled sample versus the non-labeled sample. In some instances, the difference is an increase, decrease or a lack of protein-probe interaction in the two samples. In some instances, the isotope-labeled method is termed SILAC, stable isotope labeling using amino acids in cell culture.

In some embodiments, a method comprises incubating a sample (e.g. cell sample, cell lysate sample, or comprising isolated proteins) with a labeling group (e.g., an isotopically labeled labeling group) to tag one or more proteins of interest for further analysis. In such cases, the detectable labeling group comprises a biotin, a streptavidin, bead, resin, a solid support, or a combination thereof, and further comprises a linker that is optionally isotopically labeled. As described above, the linker can be about 5, 6, 7, 8, 9, 10, 11, 12, 13, 14, 15 or more residues in length and might further comprise a cleavage site, such as a protease cleavage site (e.g., TEV cleavage site). In some cases, the labeling group is a biotin-linker moiety, which is optionally isotopically labeled with ¹³C and ¹⁵N atoms at one or more amino acid residue positions within the linker. In some cases, the biotin-linker moiety is a isotopically-labeled TEV-tag as previously described.¹⁰

In some embodiments, an isotopic reductive dimethylation (ReDi) method is utilized for processing a sample. In some cases, the ReDi labeling method involves reacting peptides with formaldehyde to form a Schiff base, which is then reduced by cyanoborohydride. This reaction dimethylates free amino groups on N-termini and lysine side chains and monomethylates N-terminal prolines. In some cases, the ReDi labeling method comprises methylating peptides from a first processed sample with a “light” label using reagents with hydrogen atoms in their natural isotopic distribution and peptides from a second processed sample with a “heavy” label using deuterated formaldehyde and cyanoborohydride. Subsequent proteomic analysis (e.g., mass spectrometry analysis) based on a relative peptide abundance between the heavy and light peptide version might be used for analysis of probe-protein interactions.

In some embodiments, isobaric tags for relative and absolute quantitation (iTRAQ) method is utilized for processing a sample. In some cases, the iTRAQ method is based on the covalent labeling of the N-terminus and side chain amines of peptides from a processed sample. In some cases, reagent such as 4-plex or 8-plex is used for labeling the peptides.

In some embodiments, the probe-protein complex is further conjugated to a chromophore, such as a fluorophore. In some instances, the probe-protein complex is separated and visualized utilizing an electrophoresis system, such as through a gel electrophoresis, or a capillary electrophoresis. Exemplary gel electrophoresis includes agarose based gels, polyacrylamide based gels, or starch based gels. In some instances, the probe-protein is subjected to a native electrophoresis condition. In some instances, the probe-protein is subjected to a denaturing electrophoresis condition.

In some instances, the probe-protein after harvesting is further fragmentized to generate protein fragments. In some instances, fragmentation is generated through mechanical stress, pressure, or chemical means. In some instances, the protein from the probe-protein complexes is fragmented by a chemical means. In some embodiments, the chemical means is a protease. Exemplary proteases include, but are not limited to, serine proteases such as chymotrypsin A, penicillin G acylase precursor, dipeptidase E, DmpA aminopeptidase, subtilisin, prolyl oligopeptidase, D-Ala-D-Ala peptidase C, signal peptidase I, cytomegalovirus assemblin, Lon-A peptidase, peptidase Clp, Escherichia coli phage KIF endosialidase CIMCD self-cleaving protein, nucleoporin 145, lactoferrin, murein tetrapeptidase LD-carboxypeptidase, or rhomboid-1; threonine proteases such as ornithine acetyltransferase; cysteine proteases such as TEV protease, amidophosphoribosyltransferase precursor, gamma-glutamyl hydrolase (Rattus norvegicus), hedgehog protein, DmpA aminopeptidase, papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, or DeSI-1 peptidase; aspartate proteases such as beta-secretase 1 (BACE1), beta-secretase 2 (BACE2), cathepsin D, cathepsin E, chymosin, napsin-A, nepenthesin, pepsin, plasmepsin, presenilin, or renin; glutamic acid proteases such as AfuGprA; and metalloproteases such as peptidase_M48.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC methods well known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, and aqueous normal-phase chromatography.

In some embodiments, the LC method is a high performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, and aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electro spray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method. In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more cysteine binding proteins or protein fragments disclosed herein. In some embodiments, the NMR method includes one dimensional (1D) NMR methods, two dimensional (2D) NMR methods, solid state NMR methods and NMR chromatography. Exemplary 1D NMR methods include ¹Hydrogen, ¹³Carbon, ¹⁵Nitrogen, ¹⁷Oxygen, ¹⁹Fluorine, ³¹Phosphorus, ³⁹Potassium, ²³Sodium, ³³Sulfur, ⁸⁷Strontium, ²⁷Aluminium, ⁴³Calcium, ³⁵Chlorine, ³⁷Chlorine, ⁶³Copper, ⁶⁵Copper, ⁵⁷Iron, ²⁵Magnesium, ¹⁹⁹Mercury or ⁶⁷Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state .sup.13Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the results from the mass spectroscopy method are analyzed by an algorithm for protein identification. In some embodiments, the algorithm combines the results from the mass spectroscopy method with a protein sequence database for protein identification. In some embodiments, the algorithm comprises ProLuCID algorithm, Probity, Scaffold, SEQUEST, or Mascot.

In accordance with the presently disclosed subject matter, as described above or as discussed in the EXAMPLES below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al. (1989) Molecular Cloning: A Laboratory Manual, Cold Spring Harbor Laboratory Publications, Cold Spring Harbor, N.Y., United States of America; Glover (1985) DNA Cloning: A Practical Approach. Oxford Press, Oxford; Gait (1984) Oligonucleotide Synthesis: A Practical Approach, IRL Press, Oxford, England; Harlow & Lane, 1988, Antibodies, A Laboratory Manual, Cold Spring Harbor Publications, New York; Roe et al. (1996) DNA Isolation and Sequencing: Essential Techniques, John Wiley, New York, N.Y., United States of America; and Ausubel et al. (1995) Current Protocols in Molecular Biology, Greene Publishing.

### X. Therapeutic Uses and Pharmaceutical Compositions

Small molecules, such as the presently disclosed purine-based ligands and probes, present an alternative method to selectively modulate proteins and to serve as leads for the development of novel therapeutics.

Dysregulated expression of a cysteine-containing protein, in many cases, is associated with or modulates a disease, such as an inflammatory related disease, an immune system related disease, a neurodegenerative disease, or cancer. As such, identification of a potential agonist/antagonist to a cysteine-containing protein aids in improving the disease condition in a patient.

Thus, in some embodiments, disclosed herein are cysteine-containing proteins that comprise one or more ligandable cysteines. In some embodiments, the cysteine-containing protein is selected from a protein listed in Table 3 or Table 4, below. In some embodiments, the cysteine-containing protein is selected from the group comprising the selenocysteine elongation factor (eEF-Sec), macrophage migration inhibitory factor or serine/threonine protein kinase 38-like.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the presently disclosed subject matter provides pharmaceutical compositions comprising one or more of the presently disclosed ligands or probes. The pharmaceutical compositions comprise at least one disclosed compound, e.g. selected from compounds of Formula (I), (Ia), (Ib), (III), (IIIa), (IIIb), and (III′) and related formulas described herein in combination with a pharmaceutically acceptable carrier, vehicle, or diluent, such as an aqueous buffer at a physiologically acceptable pH (e.g., pH 7 to 8.5), a non-aqueous liquid, a polymer-based nanoparticle vehicle, a liposome, and the like. The pharmaceutical compositions can be delivered in any suitable dosage form, such as a liquid, gel, solid, cream, or paste dosage form. In one embodiment, the compositions can be adapted to give sustained release of the probe.

In some embodiments, the pharmaceutical compositions include, but are not limited to, those forms suitable for oral, rectal, nasal, topical, (including buccal and sublingual), transdermal, vaginal, parenteral (including intramuscular, subcutaneous, and intravenous), spinal (epidural, intrathecal), central (intracerebroventricular) administration, in a form suitable for administration by inhalation or insufflation. The compositions can, where appropriate, be provided in discrete dosage units. The pharmaceutical compositions of the invention can be prepared by any of the methods well known in the pharmaceutical arts. Some preferred modes of administration include intravenous (i.v.), intraperitoneal (i.p.), topical, subcutaneous, and oral.

Pharmaceutical formulations suitable for oral administration include capsules, cachets, or tablets, each containing a predetermined amount of one or more of the ligands, as a powder or granules. In another embodiment, the oral composition is a solution, a suspension, or an emulsion. Alternatively, the ligands can be provided as a bolus, electuary, or paste. Tablets and capsules for oral administration can contain conventional excipients such as binding agents, fillers, lubricants, disintegrants, colorants, flavoring agents, preservatives, or wetting agents. The tablets can be coated according to methods well known in the art, if desired. Oral liquid preparations include, for example, aqueous or oily suspensions, solutions, emulsions, syrups, or elixirs. Alternatively, the compositions can be provided as a dry product for constitution with water or another suitable vehicle before use. Such liquid preparations can contain conventional additives such as suspending agents, emulsifying agents, non-aqueous vehicles (which may include edible oils), preservatives, and the like. The additives, excipients, and the like typically will be included in the compositions for oral administration within a range of concentrations suitable for their intended use or function in the composition, and which are well known in the pharmaceutical formulation art. The presently disclosed ligands will be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

Pharmaceutical compositions for parenteral, spinal, or central administration (e.g. by bolus injection or continuous infusion) or injection into amniotic fluid can be provided in unit dose form in ampoules, pre-filled syringes, small volume infusion, or in multi-dose containers, and preferably include an added preservative. The compositions for parenteral administration can be suspensions, solutions, or emulsions, and can contain excipients such as suspending agents, stabilizing agents, and dispersing agents. Alternatively, the ligands can be provided in powder form, obtained by aseptic isolation of sterile solid or by lyophilization from solution, for constitution with a suitable vehicle, e.g. sterile, pyrogen-free water, before use. The additives, excipients, and the like typically will be included in the compositions for parenteral administration within a range of concentrations suitable for their intended use or function in the composition, and which are well known in the pharmaceutical formulation art. The ligands of the presently disclosed subject matter can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 100 millimolar, preferably at least about 1 nanomolar to about 10 millimolar.

Pharmaceutical compositions for topical administration of the ligands to the epidermis (mucosal or cutaneous surfaces) can be formulated as ointments, creams, lotions, gels, or as a transdermal patch. Such transdermal patches can contain penetration enhancers such as linalool, carvacrol, thymol, citral, menthol, t-anethole, and the like. Ointments and creams can, for example, include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels preferably include an aqueous carrier base and include a gelling agent such as cross-linked polyacrylic acid polymer, a derivatized polysaccharide (e.g., carboxymethyl cellulose), and the like. The additives, excipients, and the like typically will be included in the compositions for topical administration to the epidermis within a range of concentrations suitable for their intended use or function in the composition, and which are well known in the pharmaceutical formulation art. The ligands of the presently disclosed subject matter can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

Pharmaceutical compositions suitable for topical administration in the mouth (e.g., buccal or sublingual administration) include lozenges comprising the ligand in a flavored base, such as sucrose, acacia, or tragacanth; pastilles comprising the ligand in an inert base such as gelatin and glycerin or sucrose and acacia; and mouthwashes comprising the active ingredient in a suitable liquid carrier. The pharmaceutical compositions for topical administration in the mouth can include penetration enhancing agents, if desired. The additives, excipients, and the like typically will be included in the compositions of topical oral administration within a range of concentrations suitable for their intended use or function in the composition, and which are well known in the pharmaceutical formulation art. The ligands of the presently disclosed subject matter invention can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

A pharmaceutical composition suitable for rectal administration comprises a ligand of the presently disclosed subject matter in combination with a solid or semisolid (e.g., cream or paste) carrier or vehicle. For example, such rectal compositions can be provided as unit dose suppositories. Suitable carriers or vehicles include cocoa butter and other materials commonly used in the art. The additives, excipients, and the like typically will be included in the compositions of rectal administration within a range of concentrations suitable for their intended use or function in the composition, and which are well known in the pharmaceutical formulation art. The ligands of the presently disclosed subject matter can be included in the compositions within a therapeutically useful and effective concentration range, as determined by routine methods that are well known in the medical and pharmaceutical arts. For example, a typical composition can include one or more of the ligands at a concentration in the range of at least about 0.01 nanomolar to about 1 molar, preferably at least about 1 nanomolar to about 100 millimolar.

According to one embodiment, pharmaceutical compositions of the present invention suitable for vaginal administration are provided as pessaries, tampons, creams, gels, pastes, foams, or sprays containing a ligand of the presently disclosed subject matter in combination with a carriers as are known in the art. Alternatively, compositions suitable for vaginal administration can be delivered in a liquid or solid dosage form. The additives, excipients, and the like typically will be included in the compositions of vaginal administration within a range of concentrations suitable for their intended use or function in the composition, and which are well known in the pharmaceutical formulation art. The ligands of the presently disclosed subject matter will be included in the compositions within a therapeutically useful and effective concentration range, as determined by routine methods that are well known in the medical and pharmaceutical arts. For example, a typical composition can include one or more of the presently disclosed ligands at a concentration in the range of at least about 0.01 nanomolar to about 1 molar, preferably at least about 1 nanomolar to about 100 millimolar.

Pharmaceutical compositions suitable for intra-nasal administration are also encompassed by the present invention. Such intra-nasal compositions comprise a ligand of the presently disclosed subject matter in a vehicle and suitable administration device to deliver a liquid spray, dispersible powder, or drops. Drops may be formulated with an aqueous or non-aqueous base also comprising one or more dispersing agents, solubilizing agents, or suspending agents. Liquid sprays are conveniently delivered from a pressurized pack, an insufflator, a nebulizer, or other convenient means of delivering an aerosol comprising the ligand. Pressurized packs comprise a suitable propellant such as dichlorodifluoromethane, trichlorofluoromethane, dichlorotetrafluoroethane, carbon dioxide, or other suitable gas as is well known in the art. Aerosol dosages can be controlled by providing a valve to deliver a metered amount of the ligand. Alternatively, pharmaceutical compositions for administration by inhalation or insufflation can be provided in the form of a dry powder composition, for example, a powder mix of the ligand and a suitable powder base such as lactose or starch. Such powder composition can be provided in unit dosage form, for example, in capsules, cartridges, gelatin packs, or blister packs, from which the powder can be administered with the aid of an inhalator or insufflator. The additives, excipients, and the like typically will be included in the compositions of intra-nasal administration within a range of concentrations suitable for their intended use or function in the composition, and which are well known in the pharmaceutical formulation art. The ligand of the presently disclosed subject matter will be included in the compositions within a therapeutically useful and effective concentration range, as determined by routine methods that are well known in the medical and pharmaceutical arts. For example, a typical composition can include one or more ligand at a concentration in the range of at least about 0.01 nanomolar to about 1 molar, preferably at least about 1 nanomolar to about 100 millimolar.

Optionally, the pharmaceutical compositions of the presently disclosed subject matter can include one or more other therapeutic agent, e.g., as a combination therapy. The additional therapeutic agent will be included in the compositions within a therapeutically useful and effective concentration range, as determined by routine methods that are well known in the medical and pharmaceutical arts. The concentration of any particular additional therapeutic agent may be in the same range as is typical for use of that agent as a monotherapy, or the concentration can be lower than a typical monotherapy concentration if there is a synergy when combined with a ligand of the presently disclosed subject matter.

### XI. Kits/Articles of Manufacture

Disclosed herein, in certain embodiments, are kits and articles of manufacture for use with one or more methods described herein. In some embodiments, described herein is a kit for generating a protein comprising a detectable group and/or a fragment of a ligand compound described herein. In some embodiments, such kit includes a probe or ligand as described herein, small molecule fragments or libraries, and/or controls, and reagents suitable for carrying out one or more of the methods described herein. In some instances, the kit further comprises samples, such as a cell sample, and suitable solutions such as buffers or media. In some embodiments, the kit further comprises recombinant proteins for use in one or more of the methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use. For example, the container(s) include probes, ligands, control compounds, and one or more reagents for use in a method disclosed herein.

The presently disclosed kits and articles of manufacture optionally include an identifying description or label or instructions relating to its use in the methods described herein. For example, a kit typically includes labels listing contents and/or instructions for use, and package inserts with instructions for use. A set of instructions will also typically be included. In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Example 1

### Synthetic Methods and Compound Characterization

### Preparation of Pu-1 (for Synthesis of N7 Tautomer):

Into a 250 mL round bottom flask was placed 2,6-dichloropurine (5.0 g, 26.5 mmol), dry THF (110 mL) and a stir bar. The reaction was placed under nitrogen and treated with methylmagnesium chloride (MeMgCl, 3.0 M in THF, 9.7 mL, 29.1 mmol). After 30 minutes the reaction was treated with propargyl bromide (80 wt % in toluene, 8.84 ml, 79.4 mmol). The reaction was then heated to 70 C in an oil bath. After 17 hours the reaction was cooled and treated with methanol (25 mL) and concentrated on the rotovap. The residue was re-dissolved in DCM (100 mL) and re-concentrated to give a solid. This material was purified on a single cartridge flash purification system sold under the tradename ISOLERA™ One (Biotage, Uppsala, Sweden) using 5% acetone to 20% acetone/chloroform as the mobile phase. This provided 1.3 grams of the desired N-7 substituted product.

### 2,6-Dichloro-7-(prop-2-yn-1-yl)-7H-purine (Pu-1)

¹H NMR (600 MHz, Chloroform-d) δ 8.48 (s, 1H), 5.27 (d, J=2.6 Hz, 2H), 2.70 (t, J=2.6 Hz, 1H). ¹³C NMR (151 MHz, DMSO-d6) δ 163.24, 151.84, 151.30, 143.40, 121.61, 78.08, 77.55, 36.62. ESI-TOF (HRMS) m/z [M+H]+ calculated for C₈H₅C₁₂N₄⁺ 226.9886, found 226.9885.

### General Procedure for Preparing Probe Compounds (N7 and N9 Tautomers):

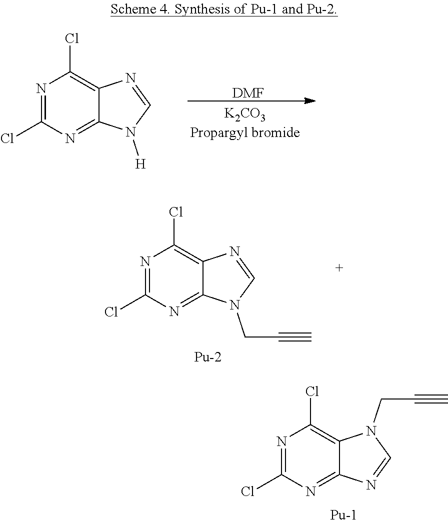

As shown in Scheme 4, above, into a 250 mL round bottom flask was placed 2,6-dichloropurine (4.10 g, 21.7 mmol), dimethylformamide (DMF, 100 mL), potassium carbonate (K₂CO₃, 3.00 grams, 21.7 mmol) and propargyl bromide (80 wt % in toluene, 2.4 ml, 21.7 mmol). The reaction mixture was stirred under nitrogen at room temperature for 12 hours. The reaction was partitioned between ethyl acetate and water (200 mL each). The layers were separated and the aqueous layer was extracted with ethyl acetate (2×100 ml). The combined organic layer was dried over sodium sulfate and concentrated to a tan solid (5.3 g). This was dissolved in chloroform (100 mL). The solution was concentrated to approximately 20 mL and heated to reflux to dissolve all the solids. Upon cooling a white crystalline solid formed which was isolated by filtration. The solid was rinsed with fresh chloroform (20 mL) and heptane (20 mL) to give 1.15 g of the N-9 substituted product after air drying. The filtrate contained a mixture of the N-7 and N-9 products. These were separated on a single cartridge flash purification system sold under the tradename ISOLERA™ One (Biotage, Uppsala, Sweden) using 5% acetone to 20% acetone/chloroform as the mobile phase to give 550 mg and 1.55 g, respectively.

### 2,6-Dichloro-9-(prop-2-yn-1-yl)-9H-purine (Pu-2)

¹H NMR (600 MHz, Chloroform-d) δ 8.33 (s, 1H), 5.04 (d, J=2.6 Hz, 2H), 2.61 (t, J=2.6 Hz, 1H). ¹³C NMR (151 MHz, DMSO-d₆) δ 152.91, 151.21, 149.89, 147.71, 130.44, 77.07, 76.92, 33.52. ESI-TOF (HRMS) m/z [M+H]+ calculated for C₈H₅C₁₂N₄⁺ 226.9886, found 226.9887.

Other purine probes were prepared using the same method using different halogentated purines as the starting material in place of 2,6-dichloropurine.

### 6-Chloro-7-(prop-2-yn-1-yl)-7H-purine (Pu-3)

¹H NMR (600 MHz, Chloroform-d) δ 8.90 (s, 1H), 8.48 (s, 1H), 5.30 (d, J=2.6 Hz, 2H), 2.68 (t, J=2.6 Hz, 1H). ¹³C NMR (151 MHz, DMSO-d₆) δ 161.59, 152.05, 150.34, 142.43, 121.81, 77.93, 77.76, 36.47. ESI-TOF (HRMS) m/z [M+H]+ calculated for C₈H₆ClN₄⁺ 193.0276, found 193.0276.

### 6-Chloro-9-(prop-2-yn-1-yl)-9H-purine (Pu-4)

¹H NMR (600 MHz, Chloroform-d) δ 8.78 (s, 1H), 8.34 (s, 1H), 5.07 (d, J=2.6 Hz, 2H), 2.59 (t, J=2.6 Hz, 1H). ¹³C NMR (151 MHz, DMSO-d₆) δ 151.82, 151.39, 149.21, 146.80, 130.73, 77.30, 76.71, 33.27. ESI-TOF (HRMS) m/z [M+H]+ calculated for C₈H₆ClN₄⁺ 193.0276, found 193.0275.

### 2-Chloro-7-(prop-2-yn-1-yl)-7H-purine (Pu-5)

¹H NMR (600 MHz, DMSO-d₆) δ 9.19 (s, 1H), 8.84 (s, 1H), 5.35 (d, J=2.6 Hz, 2H), 3.69-3.67 (m, 1H). ¹³C NMR (151 MHz, DMSO-d₆) δ 162.28, 153.15, 150.62, 143.42, 124.25, 77.77, 76.88, 35.42. ESI-TOF (HRMS) m/z [M+H]+ calculated for C₈H₆ClN₄⁺ 193.0276, found 193.0276.

### 2-Chloro-9-(prop-2-yn-1-yl)-9H-purine (Pu-6)

¹H NMR (600 MHz, DMSO-d₆) δ 9.13 (s, 1H), 8.72 (s, 1H), 5.17 (d, J=2.5 Hz, 2H), 3.57 (t, J=2.6 Hz, 1H). ¹³C NMR (151 MHz, DMSO-d₆) δ 152.96, 152.57, 150.16, 147.43, 132.96, 77.24, 76.76, 32.86. ESI-TOF (HRMS): m/z [M+H]+ calculated for C₈H₆ClN₄⁺ 193.0276, found 193.0275.

### 2-Amino-6-chloro-9-(prop-2-yn-1-yl)-9H-purine (Pu-8)

¹H NMR (600 MHz, DMSO-d₆) δ 8.17 (s, 1H), 7.02 (s, 2H), 4.93 (d, J=2.5 Hz, 2H), 3.48 (t, J=2.5 Hz, 1H). ¹³C NMR (151 MHz, DMSO-d₆) δ 159.92, 153.56, 149.51, 142.34, 123.06, 77.89, 76.08, 32.40. ESI-TOF (HRMS) m/z [M+H]+ calculated for C₈H₇ClN5⁺ 208.0384, found 208.0384.

### 6-chloro-2-fluoro-9-(prop-2-yn-1-yl)-9H-purine (Pu-10)

¹H NMR (600 MHz, DMSO-d₆) δ 8.75 (s, 1H), 5.16 (d, J=2.6 Hz, 2H), 3.60 (t, J=2.6 Hz, 1H). ¹³C NMR (151 MHz, DMSO-d₆) δ 156.99, 155.57, 153.42, 150.67, 147.66, 129.93, 77.00, 76.88, 33.45. ESI-TOF (HRMS): m/z [M+H]+ calculated for C₈H₅ClFN₄⁺ 211.0181, found 211.0182.

### General Procedure for the Preparation of 1-Alkylthiol Adducts:

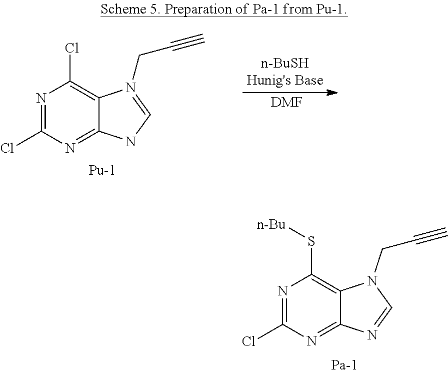

As shown in Scheme 5, above, into a 50 mL round bottom flask was placed the dichloropurine compound (831 mg, 3.66 mmol), DMF (10 mL), potassium carbonate (powdered, 556 mg, 4.03 mmol) and n-butanethiol (373 mg, 4.14 mmol). The reaction was stirred under nitrogen at ambient temperature for 16 hours. The reaction was partitioned between ethyl acetate and water (25 mL/40 mL). The layers were separated and the aqueous layer was extracted with ethyl acetate (2×25 mL). The combined organic layer was washed with water (25 mL) and brine (25 mL). It was then dried over magnesium sulfate and concentrated to give crude product that was purified on a single cartridge flash purification system sold under the tradename ISOLERA™ One (Biotage, Uppsala, Sweden) to give 650 mg of product as an off-white solid.

### 6-(Butylthio)-2-chloro-7-(prop-2-yn-1-yl)-7H-purine (Pa-1)

¹H NMR (600 MHz, Chloroform-d) δ 8.27 (s, 1H), 5.26-5.21 (m, 2H), 3.47-3.40 (m, 2H), 2.67 (t, J=2.6 Hz, 1H), 1.82-1.72 (m, 2H), 1.51 (dq, J=14.7, 7.4 Hz, 2H), 0.98 (t, J=7.4 Hz, 3H). ESI-TOF (HRMS): m/z [M+H]+ calculated for C₁₂H₁₄ClN₄S⁺ 281.0622, found 281.0621.

The other adducts were prepared via analogous reactions using other probes as the starting materials. For example, Pa-6 was prepared using Pu-10 as the starting material in place of the Pu-1 used in Scheme 5.

### 6-(butylthio)-2-fluoro-9-(prop-2-yn-1-yl)-9H-purine (Pa-6)

¹H NMR (600 MHz, Chloroform-d) δ 8.11 (s, 1H), 4.94 (d, J=2.6 Hz, 2H), 3.39-3.33 (m, 2H), 2.54 (t, J=2.6 Hz, 1H), 1.80-1.73 (m, 2H), 1.50 (dq, J=14.8, 7.4 Hz, 2H), 0.96 (t, J=7.4 Hz, 3H). ESI-TOF (HRMS): m/z [M+H]+ calculated for C₁₂H₁₄FN₄S⁺ 265.0918, found 265.0918.

X-Ray crystal structures were obtained for Pu-1, Pu-3, Pu-8, and Pu-10 and were in agreement with the assigned N7- or N9-substituted regioisomer structure.

### General Procedure for the Preparation of Purine Inhibitors

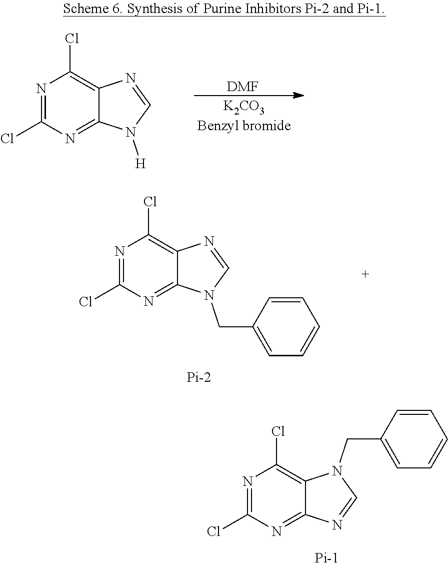

As shown in Scheme 6, above, into a 250 mL round bottom flask was placed 2,6-dichloropurine (1.16 g, 6.13 mmol), DMF (50 mL), potassium carbonate powder (848 mg, 6.13 mmol) and benzyl bromide (1.05 g, 6.13 mmol). The reaction was stirred under nitrogen at room temperature. After 16 hours the reaction was partitioned between ethyl acetate and water (100 mL each). The layers were separated and the aqueous layer was extracted with ethyl acetate (2×100 mL). The combined organic layer was dried over MgSO₄ and concentrated on the rotovap (crude weight: 4.10 g). The crude compounds were purified on a single cartridge flash purification system sold under the tradename ISOLERA™ One (Biotage, Uppsala, Sweden) using 20% ethyl acetate/hexanes. This provided the N7 and N9 isomers (220 mg and 800 mg, respectively).

### 7-Benzyl-2,6-dichloro-7H-purine (P1-1)

¹H NMR (600 MHz, DMSO-d₆) δ 9.05 (s, 1H), 7.36 (t, J=7.3 Hz, 2H), 7.31 (t, J=7.3 Hz, 1H), 7.21 (d, J=6.9 Hz, 2H), 5.74 (s, 2H). ¹³C NMR (151 MHz, DMSO-d₆) δ 163.38, 152.87, 151.09, 143.19, 136.38, 128.82, 127.94, 126.55, 121.89, 49.54. ESI-TOF (HRMS) m/z [M+H]+ calculated for C₁₂H₉C₁₂N₄⁺ 279.0198, found 279.0197.

### 9-Benzyl-2,6-9H-purine (Pi-2)

¹H NMR (600 MHz, DMSO-d₆) δ 8.85 (s, 1H), 7.39-7.29 (m, 5H), 5.50 (s, 2H). ¹³C NMR (151 MHz, DMSO-d₆) δ 153.37, 151.11, 149.79, 148.40, 135.61, 130.49, 128.80, 128.09, 127.59, 47.07. ESI-TOF (HRMS) m/z [M+H]+ calculated for C₁₂H₉C₁₂N₄⁺ 279.0198, found 279.0199.

Other purine inhibitor compounds were prepared via analogous reactions using different halides or sulfonyl halides in place of the benzyl bromide used in Scheme 6.

### 7-Allyl-2,6-dichloro-7H-purine (P1-3)

¹H NMR (600 MHz, DMSO-d₆) δ 8.89 (s, 1H), 6.17-6.09 (m, 1H), 5.24 (d, J=10.5 Hz, 1H), 5.12 (dt, J=5.0, 1.7 Hz, 2H), 4.97 (d, J=17.2 Hz, 1H). ¹³C NMR (151 MHz, DMSO-d₆) δ 163.22, 152.47, 150.94, 143.16, 133.61, 121.89, 117.48, 48.51. ESI-TOF (HRMS) m/z [M+H]+ calculated for C₈H₇C₁₂N₄⁺ 229.0042, found 229.0042.

### 9-Allyl-2,6-dichloro-9H-purine (Pi-4)

¹H NMR (600 MHz, DMSO-d₆) δ 8.72 (s, 1H), 6.12-6.03 (m, 1H), 5.25 (d, J=10.4 Hz, 1H), 5.12 (d, J=17.2 Hz, 1H), 4.91 (dt, J=5.5, 1.6 Hz, 2H). ¹³C NMR (151 MHz, DMSO-d₆) δ 153.35, 150.98, 149.64, 148.37, 132.11, 130.43, 118.30, 45.90. ESI-TOF (HRMS) m/z [M+H]+ calculated for C₈H₇C₁₂N₄⁺ 229.0042, found 229.0043.

### 2,6-Dichloro-7-(4-nitrobenzyl)-7H-purine (Pi-5)

¹H NMR (600 MHz, DMSO-d₆) δ 9.07 (s, 1H), 8.19 (d, J=8.8 Hz, 2H), 7.46 (d, J=9.0 Hz, 2H), 5.90 (s, 2H). ¹³C NMR (151 MHz, DMSO-d₆) δ 163.48, 152.98, 151.23, 147.08, 144.08, 143.15, 127.73, 123.86, 121.99, 49.06. ESI-TOF (HRMS) m/z [M+H]+ calculated for C₁₂H₈Cl₂N₅O₂⁺ 324.0049, found 324.0051.

### 2,6-Dichloro-9-(4-nitrobenzyl)-9H-purine (Pi-6)

¹H NMR (600 MHz, DMSO-d₆) δ 8.87 (s, 1H), 8.21 (d, J=8.9 Hz, 2H), 7.57 (d, J=8.9 Hz, 2H), 5.68 (s, 2H). ¹³C NMR (151 MHz, DMSO-d₆) δ 153.49, 151.19, 149.87, 148.45, 147.20, 143.01, 130.60, 128.70, 123.85, 46.41. ESI-TOF (HRMS) m/z [M−H]− calculated for C₁₂H₆C₁₂N₅O₂⁻ 321.9904, found 321.9902.

### 4-((2,6-Dichloro-9H-purin-9-yl)sulfonyl)morpholine (Pi-8)

¹H NMR (600 MHz, DMSO-d₆) δ 8.93 (s, 1H), 3.69-3.66 (m, 4H), 3.44-3.40 (m, 4H). ¹³C NMR (151 MHz, DMSO-d₆) δ 152.36, 152.15, 150.84, 145.87, 131.35, 65.12, 46.13. ESI-TOF (HRMS) m/z [M−H]− calculated for C₉H₈C₁₂N₅O₃S⁻ 335.9730, found 335.9728.

### 2-(2,6-Dichloro-9H-purin-9-yl)-5-(hydroxymethyl)tetrahydrofuran-3,4-diol (Pi-10)

¹H NMR (600 MHz, DMSO-d₆) δ 8.98 (s, 1H), 5.97 (d, J=4.9 Hz, 1H), 5.60 (d, J=5.7 Hz, 1H), 5.26 (d, J=5.4 Hz, 1H), 5.08 (t, J=5.4 Hz, 1H), 4.51 (q, J=5.1 Hz, 1H), 4.20-4.15 (m, 1H), 3.99 (q, J=4.0 Hz, 1H), 3.71 (ddd, J=12.0, 5.3, 3.9 Hz, 1H), 3.59 (ddd, J=12.0, 5.5, 3.9 Hz, 1H). ¹³C NMR (151 MHz, DMSO-d₆) δ 153.06, 151.12, 149.85, 146.39, 131.01, 88.25, 85.67, 74.04, 69.83, 60.74.

### 2,6-Dichloro-7-((5,5,8,8-tetramethyl-5,6,7,8-tetrahydronaphthalen-2-yl)methyl)-7H-purine (Pi-11)

¹H NMR (600 MHz, DMSO-d₆) δ 9.07 (s, 1H), 7.31 (s, 1H), 7.27 (d, J=8.3 Hz, 1H), 6.94-6.90 (m, 1H), 5.65 (s, 2H), 1.61 (s, 4H), 1.19 (d, J=4.5 Hz, 12H). ¹³C NMR (151 MHz, DMSO-d₆) δ 163.32, 152.75, 151.04, 144.77, 144.22, 143.14, 133.14, 126.94, 125.30, 123.99, 121.80, 49.47, 34.39, 33.90, 33.73, 31.48, 31.44. ESI-TOF (HRMS) m/z [M+H]+ calculated for C₂₀H₂₃C₁₂N₄⁺ 389.1294, found 389.1296.

### 2,6-Dichloro-9-((5,5,8,8-tetramethyl-5,6,7,8-tetrahydronaphthalen-2-yl)methyl)-9H-purine (Pi-12)

¹H NMR (600 MHz, DMSO-d₆) δ 8.86 (s, 1H), 7.46 (d, J=2.0 Hz, 1H), 7.28 (d, J=8.2 Hz, 1H), 7.03 (dd, J=8.1, 2.0 Hz, 1H), 5.41 (s, 2H), 1.60 (s, 4H), 1.20 (d, J=19.9 Hz, 12H). ¹³C NMR (151 MHz, DMSO-d₆) δ 153.29, 151.04, 149.78, 148.31, 144.78, 144.34, 132.57, 130.50, 126.88, 126.28, 124.92, 47.17, 34.40, 34.38, 33.87, 33.72, 31.50, 31.44. ESI-TOF (HRMS) m/z [M+H]+ calculated for C₂₀H₂₃C₁₂N₄⁺ 389.1294, found 389.1298.

### 2,6-Dichloro-7-((4-methoxyphenyl)sulfonyl)-7H-purine (Pi-15)

¹H NMR (600 MHz, DMSO-d₆) δ 8.74 (s, 1H), 7.52 (d, J=8.8 Hz, 2H), 6.85 (d, J=8.8 Hz, 2H), 3.75 (s, 3H). ¹³C NMR (151 MHz, DMSO-d₆) δ 165.21, 152.60, 151.34, 151.10, 144.99, 131.50, 131.15, 126.31, 115.39, 56.19.

### 2,6-Dichloro-7-((4-methoxyphenyl)sulfonyl)-7H-purine (Pi-16)

¹H NMR (600 MHz, DMSO-d₆) δ 9.14 (s, 1H), 8.16 (d, J=9.1 Hz, 2H), 7.25 (d, J=9.1 Hz, 2H), 3.87 (s, 3H). ¹³C NMR (151 MHz, DMSO-d₆) δ 165.20, 152.60, 151.34, 151.10, 144.98, 131.49, 131.14, 126.31, 115.38, 56.19.

### General Procedure for the Preparation of Acyl-Modified Purine Inhibitors:

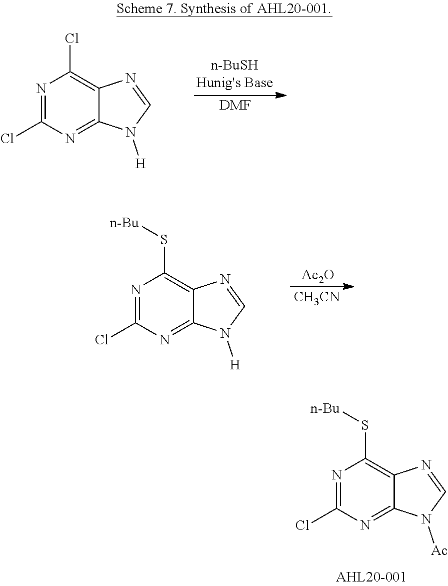

As shown in Scheme 7, above, into a 250 mL round bottom flask was placed the dichloropurine (7.57 g. 40.0 mmol DMF (100 mL), Hunig's base (8.4 mL, 48.1 mmol) and 1-butanethiol (4.72 mL, 44.1 mmol). The reaction mixture was stirred under nitrogen at ambient temperature. After 16 hours the reaction was diluted with water (150 mL) and extracted with ether (3×100 mL). The combined organic layer was washed with brine (50 mL) and dried over magnesium sulfate. This provided 1.678 grams of an off-white solid after concentration. The proton NMR showed the presence of 1-butanethiol. The solid was triturated with 30 mL of ether for 1 hours. The remaining solid was isolated by filtration to give 1.15 g of a white solid. The proton NMR indicated no remaining 1-butanethiol. NMR data was consistent with data reported in the literature.

This compound was then acylated by placing the monochloro-compound (104.5 mg, 0.431 mmol) into a 1 dram vial along with anhydrous acetonitrile (2 mL) and acetic anhydride (81.4 uL, 0.861 mmol). The reaction mixture was heated to 75° C. under nitrogen for 16 hours. The reaction was concentrated and the residue was purified on a single cartridge flash purification system sold under the tradename ISOLERA™ One (Biotage, Uppsala, Sweden; 4 g silica column, 10% EtOAc/Hexanes to 65% EtOAc/Hexanes). This provided 104.3 mg of product as a white crystalline solid (85% yield).

### 1-(6-(Butylthio)-2-chloro-9H-purin-9-yl)ethan-1-one (AHL20-001)

¹H NMR (600 MHz, DMSO-d₆): δ 8.89 (s, 1H), 3.32 (t, J=7.3 Hz, 2H), 2.81 (s, 3H), 1.68 (m, 2H), 1.41 (m, 2H), 0.90 (t, J=7.33 Hz, 3H). ¹³C NMR (150 MHz, DMSO-d₆) δ 167.9, 163.8, 153.7, 149.3, 143.5, 131.5, 31.2, 28.6, 25.3, 21.7, 13.9. ESI-QTOF (HRMS) m/z [M+H]+ calculated for C₁₁H₁₄ClN₄OS⁺ 285.0571, found 285.0576

ESI-QTOF Method (high-resolution mass spectrometry, HRMS): Compounds were dissolved in either methanol or acetone (Pi-6 and Pi-8) (500 ng/mL) and filtered through 0.2 um teflon syringe filters. Compounds were analyzed using a 1260 Infinity II LC with an Agilent 6545 Q-TOF MS (Agilent Technologies, Santa Clara, Calif., United States of America). An atmospheric pressure chemical ionization (APCI source) was utilized for analysis. Analytes were separated using an Agilent ZORBAX™ RRHD Eclipse Plus C18, 2.1 mm ID×50 mm L, 1.8 um particle diameter, 95 angstrom pore size column with 99.9% MeOH+0.1% formic acid (0.4 mL/min flow rate, 40 C column compartment). Data acquisition occurred for 1 minute. MS acquisition used the following parameters: Gas temperature: 325° C.; Vaporizer temperature: 350° C.; Dry gas: 10 L/min; Nebulizer: 60PSI; Corona Voltage: 4 uA; Vcap: 3500V; Fragmentor: 180V; Skimmer: 45V; October 1 RFVpp: 750V; Acquisition: 50-1700 m/z; Rate: 3 spectra/sec; Time: 333.3 ms/spectra; Transients/spectrum: 2665. Positive Mode Deviations: A) 5 μL injection B) Lock masses: 121.050873 & 922.009798; Negative Mode Deviation: A) 10 μL injection B) Lock masses 119.03632 & 966.000725.

HPLC assay for profiling solution purity of purine probes, ligands and fragments: The following reagents were prepared and stored on ice prior to use. 0.1 M solution of caffeine in acetonitrile, 1 M HOAc in ACN and 10 mM solution of purine compound in ACN mixture. 500 μL of purine solution were transferred to a dram vial on ice. 50 μL aliquots were removed quenched with 10 μL of a 1:1 mixture of caffeine and HOAc. Samples were injected (1 μL) and analyzed by reverse-phase HPLC on a Shimadzu 1100 Series spectrometer with UV detection at 254 nm. Chromatographic separation was performed using a Phenomenex Kinetex C18 column (2.6 μm, 50×4.6 mm). Mobile phases A and B were composed of H₂O+0.1% HOAc and ACN+0.1% HOAc, respectively. Samples were analyzed using the following analytical conditions: using a flow rate of 0.8 mL min⁻¹, the gradient was as follows: 0-0.5 min, 15% B; 0.5-6.5 min 85% B; 6.5-7 min 100% B; 7-8.5 min 100% B; 8.5-9 min 15% B; 9-9.8 min 15% B. Note: Probes (i.e Pu-compounds) were not spiked with caffeine but were rather diluted with 10 uL of ACN.

The purities of the compounds as determined by HPLC were as follows:

For the probes (Pu compounds): Pu-1, Pu-2, Pu-3, Pu-4, Pu-5, Pu-7, Pu-8 were all greater than 99% pure; Pu-6 was greater than 95% pure; Pu-9 was greater than 97% pure; and Pu-10 was greater than 98% pure.

For the ligands (Pi compounds): Pi-1 and Pi-12 were each greater than 98% pure; Pi-2, Pi-4, Pi-5, Pi-6 and Pi-10 were each greater than 99% pure; Pi-3 was greater than 95% pure; and Pi-13 was greater than 97% pure.

For the fragments (Pa compounds): Pa-1 was greater than 98% pure and Pa-3 was greater than 95% pure. For AHL20-001, purity was determined to be greater than 96%.

### Example 2

### Probe Reactivity and Stability

HPLC assay for profiling solution reactivity and stability of purine fragments: The following reagents were prepared and stored on ice prior to use. 0.1 molar (M) solution of caffeine in acetonitrile, 1.0 M solution of amino acid mimetic (butanethiol—cysteine mimetic; n-butylamine—lysine mimetic; p-cresol—tyrosine mimetic; propionamide-asparagine/glutamine mimetic; butyric acid—aspartic/glutamic acid mimetic), tetramethylguanidine (TMG), 1 M acetic acid (HOAc) in acetonitrile (ACN) and 10 mM solution of purine fragment in ACN mixture. 500 μL of fragment solution were transferred to a dram vial on ice. To the mixture, 5.5 μL of TMG and 5.5 μL of respective amino acid mimetic were added and solutions were stirred on ice for 6 h. To monitor reactivity, 50 μL aliquots were removed at indicated time points and quenched with 10 μL of a 1:1 mixture of caffeine and HOAc. Samples were injected (1 μL) and analyzed by reverse-phase HPLC on a Shimadzu 1100 Series spectrometer (Shimadzu Corporation, Kyoto, Japan) with UV detection at 254 nm. Reaction progress was evaluated by monitoring consumption of starting material (purine fragment) normalized to caffeine standard. Chromatographic separation was performed using a Phenomenex Kinetex C18 column (2.6 μm, 50×4.6 mm; Phenomenex, Torrance, Calif., United States of America). Mobile phases A and B were composed of H₂O+0.1% HOAc and ACN+0.1% HOAc, respectively. Samples were analyzed using the following analytical conditions: using a flow rate of 0.8 mL min⁻¹, the gradient was as follows: 0-0.5 min, 15% B; 0.5-6.5 min 85% B; 6.5-7 min 100% B; 7-8.5 min 100% B; 8.5-9 min 15% B; 9-9.8 min 15% B. The amount of purine fragment consumed was calculated using the area under the curve (AUC) for the fragment peak at time (t)=experimental/t=0. All purine fragment peak AUCs used for calculations were normalized to caffeine standard AUCs at respective time points to account for run-to-run variations by HPLC. The amount of purine fragment consumed (% starting material) was plotted as a function of time.

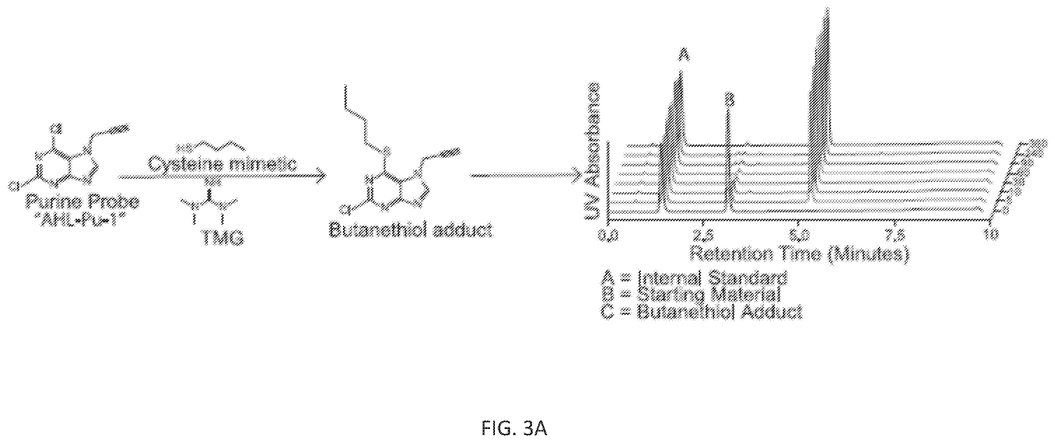

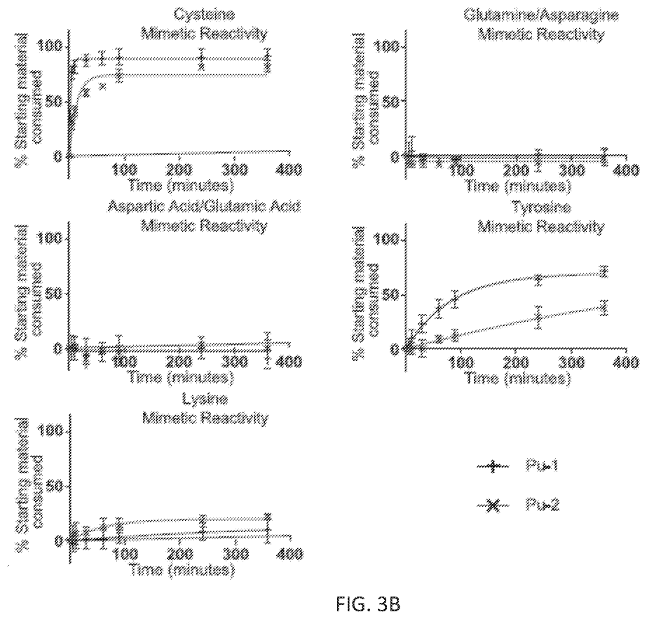

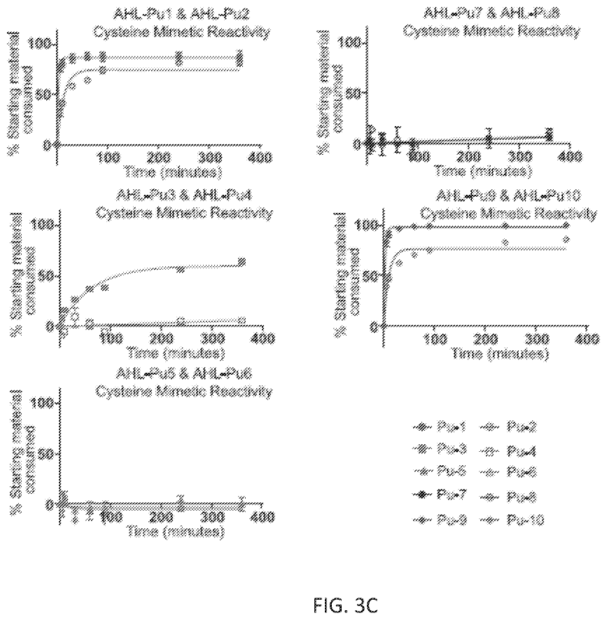

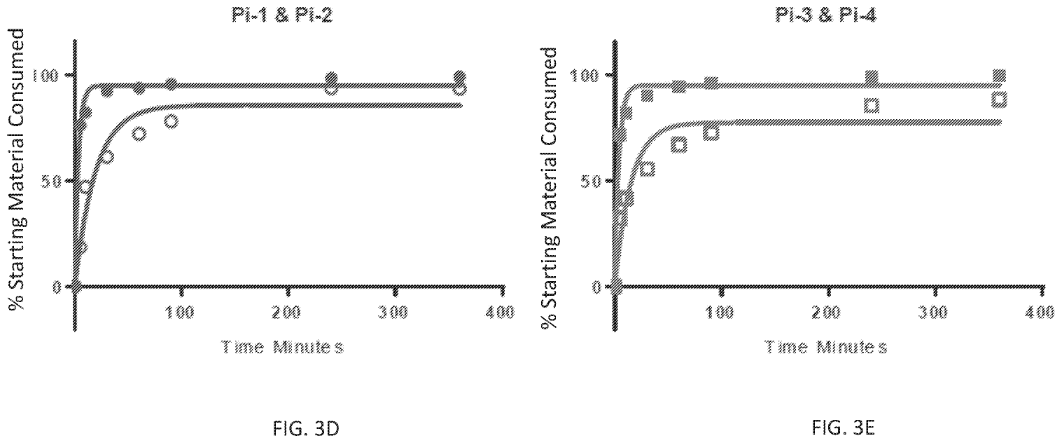

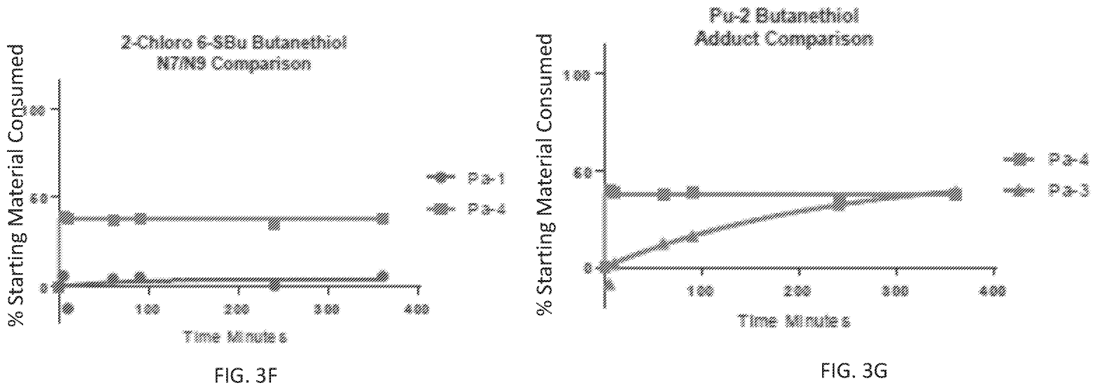

Discussion: An HPLC assay for determining the reactivity of the purine-based probes with mimetics of amino acid residues was performed. FIG. 3A shows the assay using butanethiol as a small molecule mimetic for cysteine. Reaction progress was monitored by monitoring the disappearance of starting material and appearance of product via HPLC.

Results of the HPLC analysis of the solution-based reactions of the purine-based probes Pu-1-Pu-10 with amino acid mimetics are shown in FIGS. 3B and 3C. As shown in FIG. 3B, Pu-1 modifies cysteine (Cys) and tyrosine (Tyr) amino acid mimetics in solution-based studies, while Pu-2 also exhibits mild lysine (Lys) reactivity. These probes are unreactive with Asp/Glu amino acid mimetic butyric acid and Gln/Asn amino acid mimetic propionamide. In both cases, very little starting material mimetic is consumed in 6 hours. The observed reaction kinetics support Cys chemoselectivity for halogenated purines. The N7 tautomer (Pu-1) is more reactive towards nucleophilic attack compared with the N9 counterpart (Pu-2).

As shown in FIG. 3C, Pu-1, Pu-2, Pu-3, Pu-9 and Pu-10 all react with butanethiol, while other probes do not. N7 tautomers (Pu-1, Pu-3, Pu-9) of purine molecules display accelerated reaction kinetics compared to N9 counterparts (Pu-2, Pu-4, Pu-10). Based on the HPLC traces, it appears that Pu-9 and Pu-10 likely form many different adducts (i.e. addition at the 2 position, the 6 position, and dual modification at 2 and 6). The position of the halogen leaving group affects reactivity: the 6 position enhances reactivity compared to the leaving group on the 2 position (Pu-3 versus Pu-5 and Pu-4 versus Pu-6). The addition of an amine group at the 2 position dramatically reduces the electrophilic character of N7 (Pu-7) and N9 (Pu-8) halogenated purines.

### Example 3

### Inhibitor Reactivity and Stability

HPLC assay for profiling solution reactivity and stability of purine inhibitors—The following reagents were prepared and stored on ice prior to use. 0.1 M solution of caffeine in acetonitrile, 1.0 M solution of amino acid mimetic (butanethiol), tetramethylguanidine (TMG), 1 M HOAc in ACN and 10 mM solution of purine fragment in ACN mixture. 500 μL of fragment solution were transferred to a dram vial on ice. To the mixture, 5.5 μL of TMG and 5.5 μL of respective amino acid mimetic were added and solutions were stirred on ice for 6 h. To monitor reactivity, 50 μL aliquots were removed at indicated time points and quenched with 10 μL of a 1:1 mixture of caffeine and HOAc. Samples were injected (1 μL) and analyzed by reverse-phase HPLC on a Shimadzu 1100 Series spectrometer (Shimadzu Corporation, Kyoto, Japan) with UV detection at 254 nm. Reaction progress was evaluated by monitoring consumption of starting material (purine fragment) normalized to caffeine standard. Chromatographic separation was performed using a Phenomenex Kinetex C18 column (2.6 μm, 50×4.6 mm; Phenomenex, Torrance, Calif., United States of America). Mobile phases A and B were composed of H₂O+0.1% HOAc and ACN+0.1% HOAc, respectively. Samples were analyzed using the following analytical conditions: using a flow rate of 0.8 mL min⁻¹, the gradient was as follows: 0-0.5 min, 15% B; 0.5-6.5 min 85% B; 6.5-7 min 100% B; 7-8.5 min 100% B; 8.5-9 min 15% B; 9-9.8 min 15% B. The amount of purine fragment consumed was calculated using the area under the curve (AUC) for the fragment peak at time (t)=experimental/t=0. All purine fragment peak AUCs used for calculations were normalized to caffeine standard AUCs at respective time points to account for run-to-run variations by HPLC. The amount of purine fragment consumed (% starting material) was plotted as a function of time.

Discussion: An HPLC assay analogous to that shown in FIG. 3A was used to determine the reactivity of the purine ligands for butanethiol, a mimetic of cysteine solution. In general, the N7 tautomers of purine ligands were more reactive with butanethiol nucleophile. FIGS. 3D and 3E show the comparative reactivity of Pi-1 and Pi-2 and Pi-3 and Pi-4 The N9 tautomer counterparts, while overall less reactive, permit tuning capabilities. There were some exceptions to the higher reactivity of the N7 tautomers. Pi-13 and Pi-14 had essentially equivalent reactivity for butanethiol.

### Example 4

### Solution Reactivity and Stability of Purine Adduct Fragments

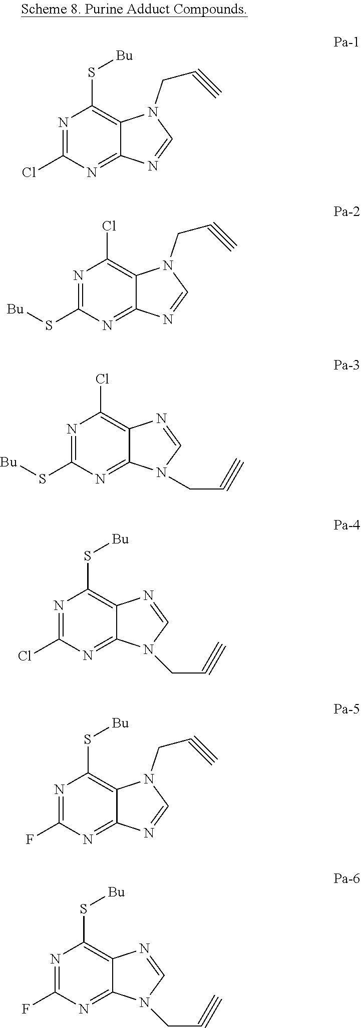

HPLC assay for profiling solution reactivity and stability of purine adduct fragments: The following reagents were prepared and stored on ice prior to use. 0.1 M solution of caffeine in acetonitrile, 1.0 M solution of amino acid mimetic (butanethiol, n-butylamine, p-cresol, propionamide and butyric acid), tetramethylguanidine (TMG), 1 M HOAc in ACN and 10 mM solution of purine fragment (e.g., one of the purine adducts shown in Scheme 8, above) in ACN mixture. 500 μL of fragment solution (i.e. purified Pa-1 through Pa-6) were transferred to a dram vial on ice. To the mixture, 5.5 μL of TMG and 5.5 μL of respective amino acid mimetic were added and solutions were stirred on ice for 6 h. To monitor reactivity, 50 μL aliquots were removed at indicated time points and quenched with 10 μL of a 1:1 mixture of caffeine and HOAc. Samples were injected (1 μL) and analyzed by reverse-phase HPLC on a Shimadzu 1100 Series spectrometer with UV detection at 254 nm. Reaction progress was evaluated by monitoring consumption of starting material (purine fragment) normalized to caffeine standard. Chromatographic separation was performed using a Phenomenex Kinetex C18 column (2.6 μm, 50×4.6 mm). Mobile phases A and B were composed of H₂O+0.1% HOAc and ACN+0.1% HOAc, respectively. Samples were analyzed using the following analytical conditions: using a flow rate of 0.8 mL min′, the gradient was as follows: 0-0.5 min, 15% B; 0.5-6.5 min 85% B; 6.5-7 min 100% B; 7-8.5 min 100% B; 8.5-9 min 15% B; 9-9.8 min 15% B. The amount of purine adduct fragment consumed was calculated using the area under the curve (AUC) for the fragment peak at time (t)=experimental/t=0. All purine fragment peak AUCs used for calculations were normalized to caffeine standard AUCs at respective time points to account for run-to-run variations by HPLC. The amount of purine fragment consumed (% starting material) was plotted as a function of time.

Results: The structures of exemplary purine adduct probe compounds are shown in Scheme 8, above. Purine adduct probes (Pa series) Pa-3, Pa-4, and Pa-6 showed mild reactivity with nucleophiles. Pa-1 showed little reactivity with either butanethiol or p-cresol. Pa-3 shows a preference for butanethiol while the other adducts show little preference. The N9 tautomer of Pa compounds (Pa-4) appears to be more reactive against nucleophiles. See FIG. 3F. The presence of the halogen group at the 2-position of 6-substituted purine compounds (Pa-4) results in higher reactivity compared with 6-halogen, 2-substituted counterparts (Pa-3). See FIG. 3G. A fluoro group at the 2-position of 6-substituted purines (Pa-6) results in equivalent reactivity against butanethiol and p-cresol.

### Example 5

### Activity of Purine Probes in Live Cells and Cell Lysates

Live Cell Activity Method: DM93 cells were grown at 37° C. in 5% CO2 until 90% confluent. Once confluent cells were washed with serum free media and treated with halogenated purine probes at a final concentration of 25 μM (unless stated) for 4 hours (unless stated). Cells were then scraped and washed 3× with cold PBS and lysed in PBS+protease inhibitor. Lysates were spun at 100,000×g for 45 minutes. Halogen probe-modified proteins in soluble fractions were visualized by conjugating rhodamine-azide using copper-catalyzed azide-alkyne cycloaddition (CuAAC; 1 hour, room temperature), subjected to SDS-PAGE, and detected by in-gel fluorescence scanning. SDS-PAGE gels were also stained with Coomassie brilliant blue to determine protein load. All samples were loaded with equivalent amounts of protein. Thus, changes in purine probe labeling is not due to loading of different proteome amounts.

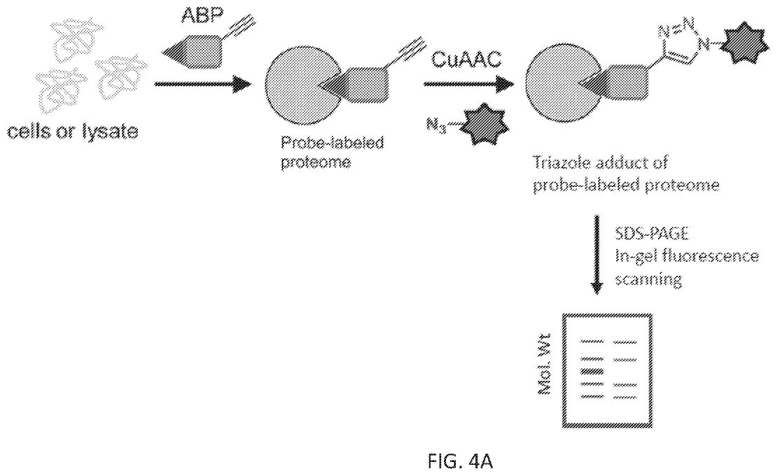

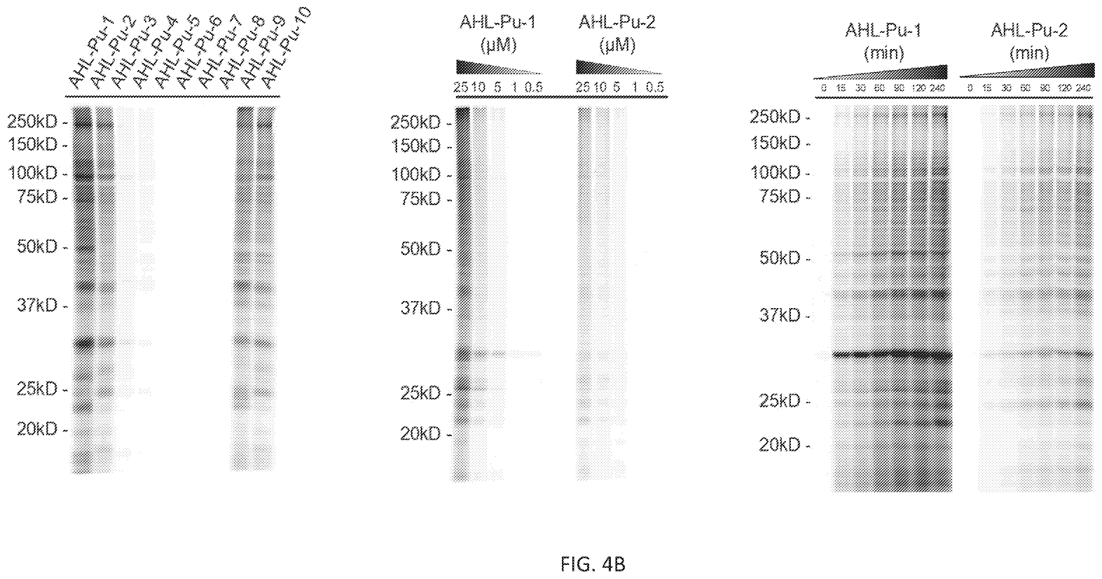

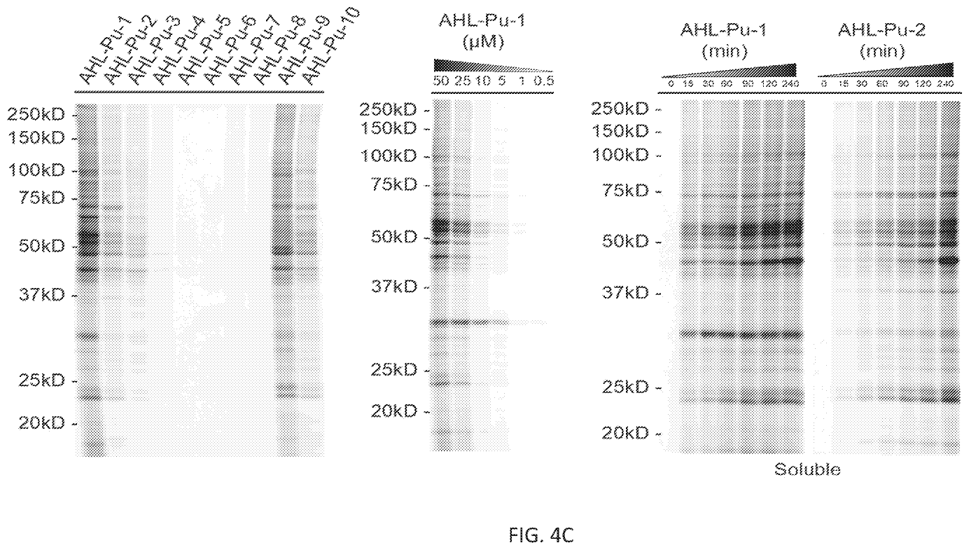

Discussion: A scheme for determining the activity of purine probes in live cells or cell lysates using gel-based analysis is shown in FIG. 4A. As shown in the left panel of FIG. 4B, AHL125 (Pu-1) AHL128 (Pu-2), 6-chloro-2-fluoro-(prop-2-yn-1-yl)-7H-purine (Pu-9) and 6-chloro-2-fluoro-(prop-2-yn-1-yl)-9H-purine (Pu-10) display robust labeling profiles in live DM93 cells. Pu-9 and Pu-10 are likely generating complex adducts (i.e. reaction with cysteine and other nucleophilic amino acids) based on HPLC reactivity data. Enhanced probe labeling with 6-chloro-(prop-2-yn-1-yl)-7H-purine (Pu-3) and 6-chloro-(prop-2-yn-1-yl)-9H-purine (Pu-4) compared to 2-chloro-(prop-2-yn-1-yl)-7H-purine (Pu-5) and 2-chloro-(prop-2-yn-1-yl)-9H-purine (Pu-6) suggest that the 6-chloro position is more electrophilic and reactive towards nucleophilic attack. Comparison between the N7 (Pu-1, Pu-3 and Pu-9) and N9 tautomers (Pu-2, Pu-4, and Pu-10) demonstrate the N7 analogs are more reactive. AHL-Pu-1 and AHL-Pu-1 show concentration dependent labeling in live cells. See FIG. 4B, middle panel. AHL-Pu-1 and AHL-Pu-2 react in a time dependent manner, which supports a covalent reaction mechanism. See FIG. 4B, right panel.

Lysate Activity Method: DM93 cells were grown at 37° C. in 5% CO2 until 90% confluent. Once confluent, cells were scraped and washed 3× times with cold PBS and lysed in PBS+protease inhibitor. Lysates were spun at 100,000×g for 45 minutes. Soluble fractions were treated with 25 μM of halogenated purine probes (unless stated) for 2 hrs (unless stated) at 37° C. Purine probe modified proteins were conjugated to rhodamine-azide by CuAAC, subjected to SDS-PAGE analysis, and detected by in-gel fluorescence scanning. SDS-PAGE gels were stained with Coomassie brilliant blue to determine protein load. All samples were loaded with equivalent amounts of protein to demonstrate changes in purine probe labeling is not due to loading of different proteome amounts.

Discussion: Similar to the live cell treatments, AHL-Pu-1, AHL-Pu-2, AHL-Pu-9 and AHL-Pu-10 show the highest protein labeling activity. See FIG. 4C, left panel. Increased labeling using AHL-Pu-3 and AHL-Pu-4 compared to AHL-Pu-5 and AHL-Pu-6 lane suggest that the 6-Chloro position is more electrophilic towards nucleophilic attack. Comparison between the N7 and N9 tautomers demonstrate the N7 analogs (AHL-Pu-1, AHL-Pu-3 and AHL-Pu-9) are more reactive. AHL-Pu-1 displays concentration dependent labeling of proteomes. See FIG. 4C, middle panel. AHL-Pu-1 and AHL-Pu-2 show time dependence in protein labeling and supports a covalent reaction mechanism with proteins in proteomes. See FIG. 4C, right panel.

### Example 6

### Activity of Purine Probes in In Vivo

Mouse Treatment and Tissue Preparation Methods: 8-12 week old male and female C57Bl/6 mice were treated intraperitoneally (IP) or by oral gavage (OG) with either AHL125 (Pu-1 or AHL-Pu-1), AHL128 (Pu-2 or AHL-Pu-2), or vehicle (18:1:2, PBS:PEG40:DMSO) at 20 mg/kg unless stated otherwise. Treatment time is indicated in the experiment below. Mice were then euthanized and perfused using PBS. Tissues were then harvested, rinsed and flash frozen using liquid nitrogen and stored at −80° C. until further use. Tissues were lysed using dounce homgenization in the presence of PBS+protease inhibitor. Initial suspensions were centrifuged at 3000×g for 5 minutes to remove insoluble material. The lysate was collected and centrifuged again at 100,000×g for 45 minutes to obtain the soluble fraction. Purine probe modified proteins from treated mice were conjugated to rhodamine-azide by CuAAC, subjected to SDS-PAGE analysis, and detected by in-gel fluorescence scanning. SDS-PAGE gels were stained with Coomassie brilliant blue to determine protein load. All samples were loaded with equivalent amounts of protein. Thus, changes in purine probe labeling is not due to loading of different proteome amounts.

Mice were also treated with 80 mg/kg oral gavage (OG) to determine whether purine probes are orally bioavailable. Mice were treated for four hours.

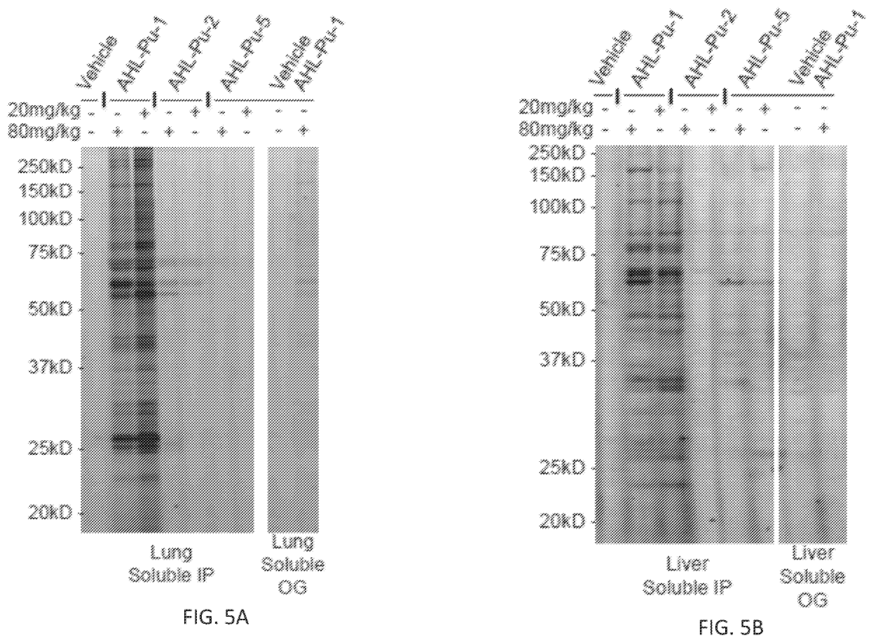

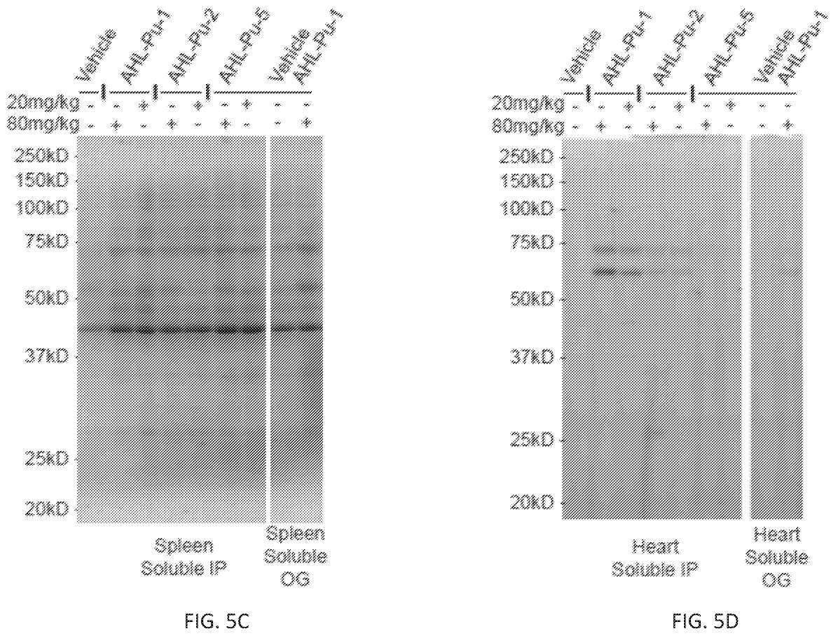

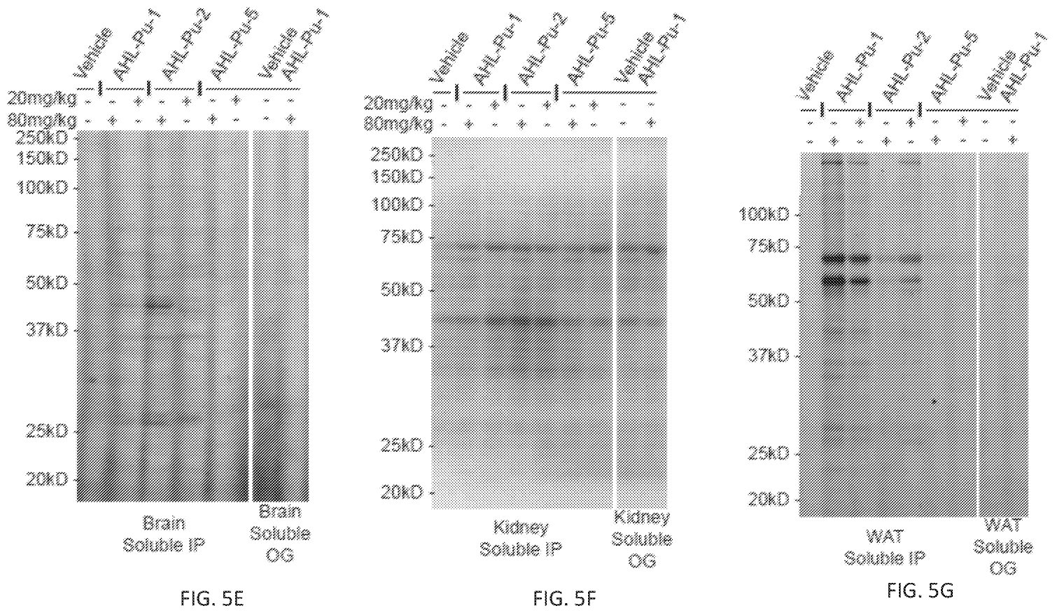

Discussion: Purine probes show concentration dependent protein labeling activity in animals. As shown in FIGS. 5A-5G, AHL125 (Pu-1) displays the most robust labeling profile in vivo across several tissues (lung, liver, spleen, heart, brain, kidney and white adipose tissue (WAT)) with highest activity in lung. These data support the hypothesis that the N7 tautomer is more electrophilic towards nucleophilic attack. Data supporting oral bioavailability of purine probes was observed in several tissues including lung and spleen. The brain shows some labeling activity, suggesting that purine probes penetrate the blood brain barrier. Pu-5 was used as a negative control.

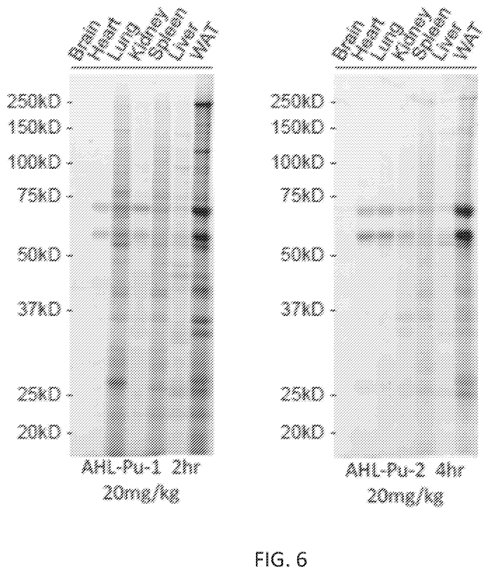

Time dependent labeling in vivo was studied using 20 mg/kg probe. In general, AHL125 (Pu-1; see FIG. 6, left panel) displayed peak labeling at 2 hours while AHL128 (Pu-2; FIG. 6, right panel) shows the most robust labeling signal at 4 hours. Tissue comparison data suggests robust labeling in the lung with some liver labeling as well. The white adipose tissues also show a very robust labeling profile. Without being bound to any one theory, these results are believed to be related to 1) site of injection (increased time and concentration) and 2) good bioavailiability due to the large amount of lipid.

### Example 7

### Purine Ligands Targeting Human Selenocysteine-Specific Elongation Factor (eEF-SEC)

Methods: HEK293T cells were grown at 37° C. in 5% CO₂ until 40% confluent. Recombinant human eEF-Sec (Uniprot ID P57772) was expressed by transient transfection for 48 hours. Afterwards, cells were scraped and washed 3× times with cold PBS and lysed in PBS+protease inhibitor. Lysates were spun at 100,000×g for 45 minutes. Soluble or membrane fractions were treated with purine probes for 2 hrs or for a predetermined period of time at 37° C. Purine probe modified proteins were conjugated to rhodamine-azide by CuAAC, subjected to SDS-PAGE analysis, and detected by in-gel fluorescence scanning. Comparison of non-transfected (Mock) and transfected proteomes was used to identify the recombinant eEF-Sec purine probe-labeled band. Purine ligand (Pi compounds) activity was evaluated by pretreating lysates with Pi ligands for a predetermined time followed by labeling with purine probe. Reduction in fluorescent signals from purine probe labeling was indicative of Pi ligand inhibitory activity. Mut represents eEF-Sec mutant where cysteine residue 442 is mutated to alanine (C₄₄₂A).

SDS-PAGE gels were transferred to nitrocellulose membrane. Nitrocellulose blots were blocked with 5% BSA. Blots were washed five times with TBS-T. Recombinant protein expression was detected using an anti-FLAG primary antibody (1:1,000) followed by fluorescent secondary antibody (1:10,000) to determine if there was equivalent protein expression across different treatment conditions.

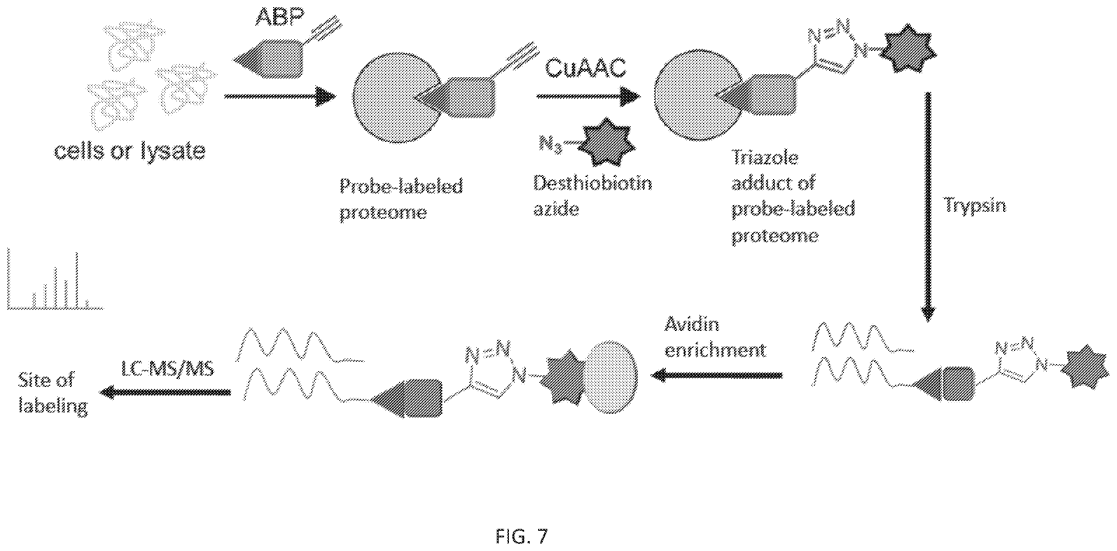

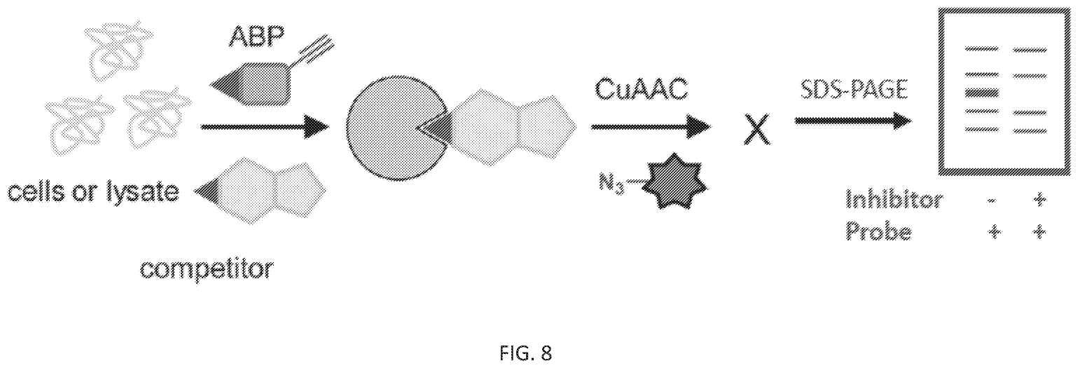

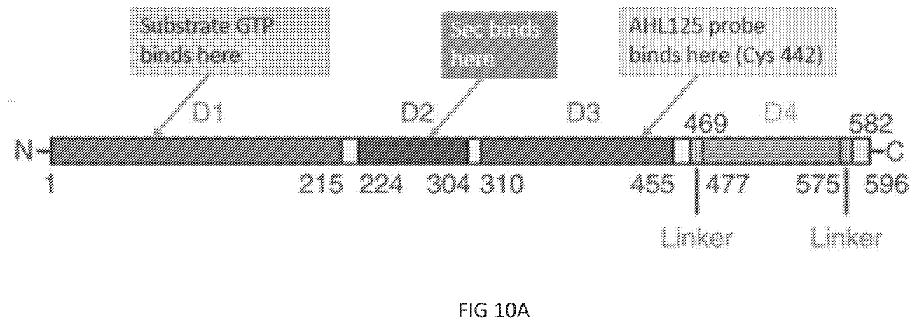

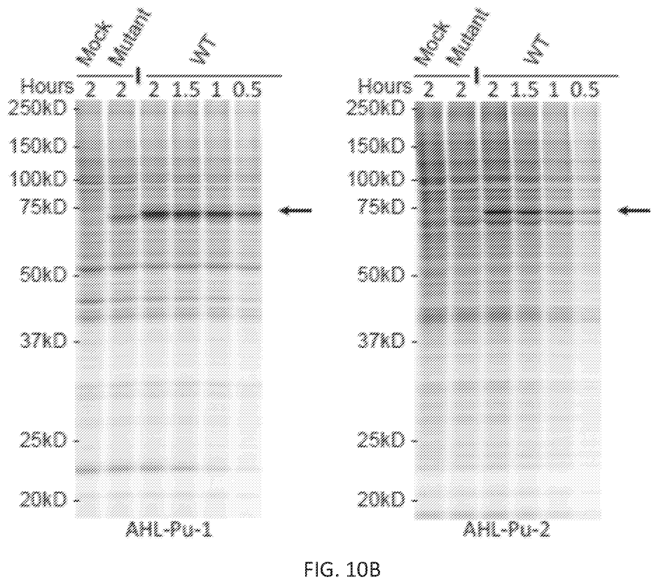

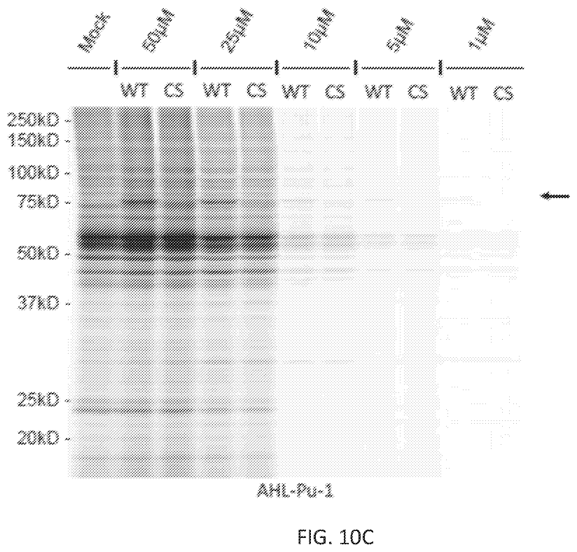

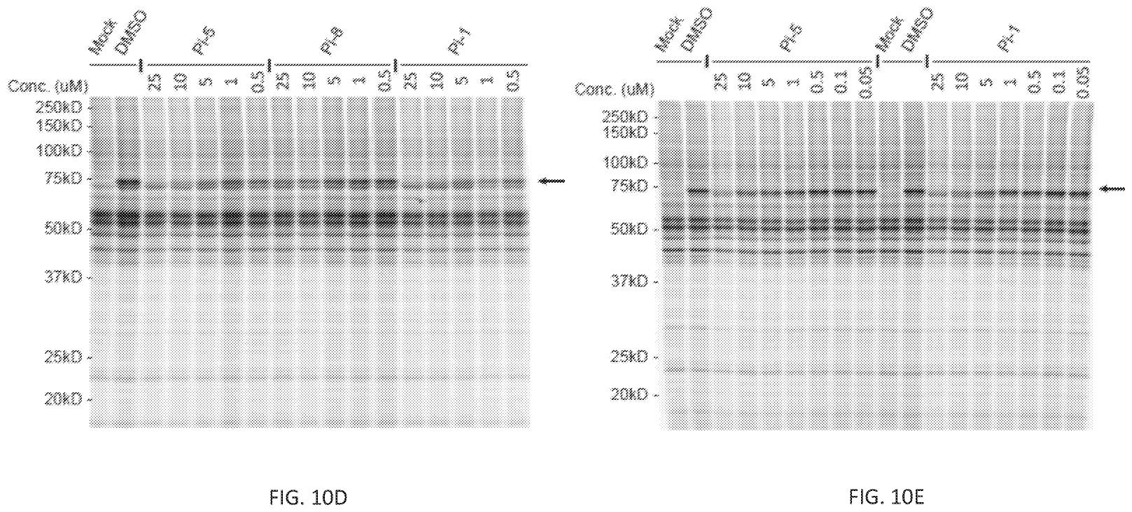

Discussion: The selenocysteine elongation factor (eEF-Sec) was identified as a target from initial LC-MS/MS experiment (see FIG. 7) using AHL125 (Pu-1). eEF-Sec is a translation factor that assists in the production of Sec-proteins (of which the human proteome contains 25). eEF-Sec is a 4 domain GTP binding protein that acts as the translation factor responsible for inserting selenocysteine (Sec) into proteins. Dysregulation of selenoproteins have been identified in a variety of disease pathologies, including cardiac, muscular, nervous system, endocrine system, immune system, and reproductive system disorders and diseases. Currently, there are no available inhibitors to study this protein. Based on the initial LC-MS/MS experiment, it appears that Pu-1 (AHL125) binds at C422 of eEF-Sec. See FIG. 10A.

A recombinant protein band at ˜65 kDa in the gels of transfected but not mock samples supported expression of eEF-Sec in HEK293T cells. Equivalent expression across different treatment conditions support changes in purine probe labeling is not due to differences in recombinant protein expression.

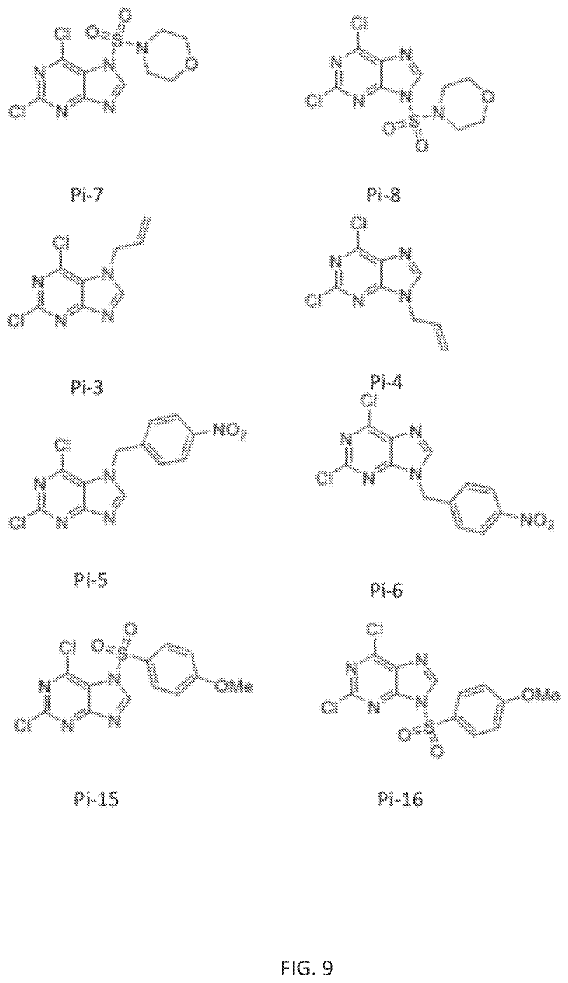

Recombinant eEF-SEC was labeled in a time dependent manner by purine probes (50 μM AHL-Pu-1 or AHL-Pu-2) in live cells. See FIG. 10B. The absence of probe labeling in the C442A EEF SEC mutant supports purine probe labeling activity at cysteine 442 site of this protein. The lack of purine probe labeling in eEF-SEC C442A mutant (CS) compared with wild-type (WT) protein at different purine probe concentrations from in vitro labeling experiments (1 hr at 37° C.) is shown in FIG. 10C. Using a competition assay (see FIG. 8), purine ligands Pi-5 and Pi-8 (10 μM, see FIG. 9) can block AHL-Pu-1 probe labeling (25 μM) in a time dependent and concentration dependent manner in live cells. See FIGS. 10D and 10E. Pi-5 shows greater than 50% inhibition at 10 μM, while its regioisomer Pi-6 loses all apparent activity against the target at the same 10 μM.

### Example 8

### LCMS Detection of Purine Protein Adducts

Methods: SILAC DM93 cells were cultured at 37° C. with 5% CO2 in either “light” or “heavy” media supplemented with 10% dialyzed fetal bovine serum (Omega Scientific), 1% L-glutamine (Fisher Scientific), and isotopically labeled amino acids. Light media was supplemented with 100 μg mL⁻¹ L-arginine and 100 μg mL⁻¹ L-lysine. Heavy media was supplemented with 100 μg mL⁻¹ [¹³C₆¹⁵N₄] L-arginine and 100 μg mL⁻¹ [¹³C₆¹⁵N₂] L-lysine. Labelled amino acids were incorporated for at least five passages before utilizing SILAC cells for experiments. Cells grown to ˜90% confluency in 10 cm plates were treated with DMSO vehicle or purine compound in serum-free media at a final concentration of 25 μM for 4 hours at 37° C. with 5% CO₂. After treatment, cells were washed with cold PBS twice before collection and preparation for chemical proteomic evaluation. Protein concentrations were normalized to 2.3 mg mL⁻¹ and 432 μL (for 1 mg final protein amount) were used for sample preparation. Probe-modified proteomes were conjugated to desthiobiotin-PEG3-azide followed by enrichment of probe-modified peptides for nano-electrospray ionization-LC-MS/MS analyses as previously described¹⁵. Identification of peptides and proteins from tandem mass spectrometry analyses was accomplished using bioinformatics software and quality control criteria as previously described¹⁵.

Discussion: Proteomes were prepared according to the methods described above. Macrophage migration inhibitor factor (MIF, Uniprot ID P14174) was selected because it passed all quality control parameters (Byonic score>300, ratio dot product [RDOTP] and isotope dot product [IDOTP]>0.8). Additionally, this protein contained a single modified Cys residue (C81) and was only observed with Pu-1 treatments. Covalent reaction with Pu-1 adds +604.2631 Da to the modified amino acid C81 from MIF and supports the proposed purine reaction mechanism whereby the halogen (Cl) serves as the leaving group during modification with nucleophilic residues on proteins.

As an additional example of a purine probe protein adduct, the protein serine/threonine-protein kinase 38-like (STK38L, Uniprot ID Q9Y2H1) was selected because it passed all quality control parameters (Byonic score>300, ratio dot product [RDOTP] and isotope dot product [IDOTP]>0.8). Additionally, this protein contained a single modified Cys residue (C235) and was only observed with Pu-1 treatment. Covalent reaction with Pu-1 adds +604.2631 Da to the modified amino acid C235 from STK38L and supports the proposed purine reaction mechanism.

### Example 9

### Quantitative Chemical Proteomics and Bioinformatics Analysis of Pu-1 and Pu-2 Modified Proteins from Lysate and Live Cell Treatments

Cells were grown at 37° C. in 5% CO₂ until 90% confluent. Once confluent cells were washed with serum free media and treated with purine probes (Pu-1, Pu-2) at a final concentration of 25 μM for 4 hours. Cells were then scraped and washed 3× with cold PBS and lysed in PBS+protease inhibitor. Lysates were spun at 100,000×g for 45 minutes. Purine probe-modified proteins in soluble fractions were coupled to desthiobiotin-azide using copper-catalyzed azide-alkyne cycloaddition (CuAAC; 1 hour, room temperature), probe-modified proteins digested into peptides using trypsin protease, probe-modified peptides enriched by avidin affinity chromatography, and subjected to LC-MS quantitative chemical proteomics as previously described.¹⁵ The following cell lines were used for analysis: DM93, Hela, A549, HEK293T, and Jurkat. All data shown are for proteins with a cysteine site modified by purine probes.

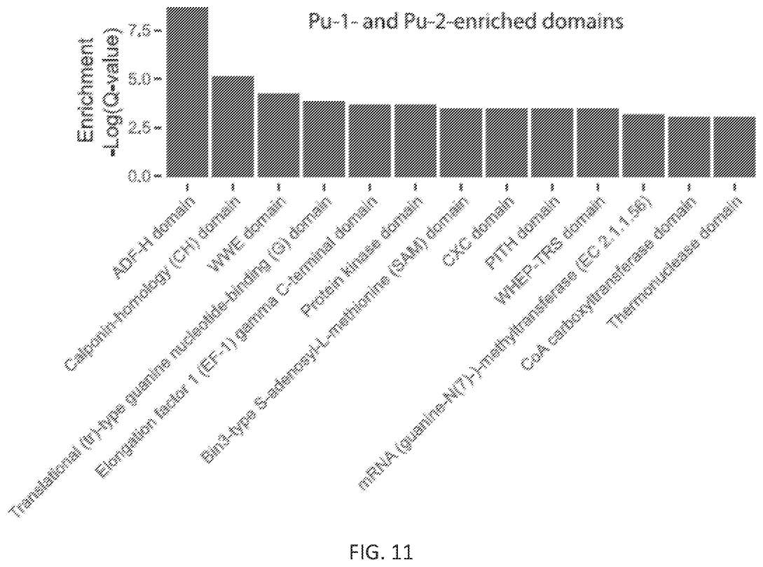

Functional protein domains that are statistically significantly enriched by Pu-1 and Pu-2 purine probes were determined by Q<0.05 after Benjamini-Hochberg correction of a two-sided binomial test following previously described methods.′⁵ Evaluation of probe-enriched domains (cysteine site on target protein) revealed enriched functions that include proteins involved in nucleotide recognition, protein ubiquitination, ADP ribosylation, and protein kinases. See FIG. 11.

Pu-1- and Pu-2-modified proteins (cysteine site) were compared with DrugBank proteins (DBP proteins). Only twenty-six percent of the purine-modified proteins (137/525) were DPB proteins. The DBP proteins were subdivided into proteins with associated compounds that are FDA-approved drugs. Twenty percent of the purine-modified proteins were proteins with associated FDA-approved drugs. Non-DBP proteins are proteins that did not match a DrugBank entry. A large fraction of purine-modified proteins (74%, 388/525) were non-DBP proteins, and thus lack pharmacological probes and/or drugs.

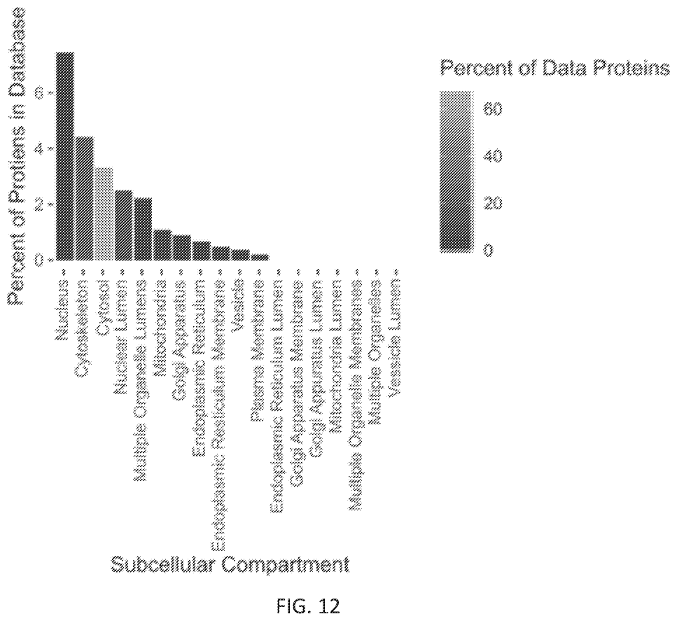

Subcellular location analysis of Pu-1- and Pu-2-modified proteins from live cell studies was performed. Proteins with a modified cysteine site were grouped based on subcellular location using a published subcellular location analysis (SLA) algorithm.′ The analysis is summarized in the graph shown in FIG. 12, where the number of modified proteins compared with the number of proteins from the SwissProt database for each subcellular compartment (x-axis) using SLA analyses are shown (Proteins in Database, y-axis). The shading in the bars depicts the percentage of modified proteins from each subcellular compartment compared with all modified proteins quantified in datasets.

Tables 1 and 2, below, show the distribution of Pu-1- and Pu-2-modified sites (high confidence sites; Byonic score>300) among the nucleophilic amino acid residues detected in proteomes. Purine probes were chemoselective for cysteine residues on target proteins (˜80% of all purine probe-modified peptides).

TABLE 1
Amino Acid Selectivity of AHL125 (Pu-1).

| Modified Amino | Number of Unique | Percentage of |
|---|---|---|
| Acid Residue | Modified Residues | Modified Residues |
| Cysteine (C) | 196 | 82 |
| Aspartic Acid (D) | 9 | 3.8 |
| Glutamic Acid (E) | 12 | 5.0 |
| Histidine (H) | 1 | 0.42 |
| Lysine (K) | 4 | 1.7 |
| Methionine (M) | 2 | 0.84 |
| Asparagine (N) | 0 | 0 |
| Glutamine (Q) | 3 | 1.3 |
| Arginine (R) | 2 | 0.84 |
| Serine (S) | 1 | 0.42 |
| Threonine (T) | 2 | 0.84 |
| Tryptophan (W) | 1 | 0.42 |
| Tyrosine (Y) | 6 | 2.5 |

TABLE 2
Amino Acid Selectivity of AHL128 (Pu-2).

| Modified Amino | Number of Unique | Percentage of |
|---|---|---|
| Acid Residue | Modified Residues | Modified Residues |
| Cysteine (C) | 952 | 80 |
| Aspartic Acid (D) | 44 | 3.7 |
| Glutamic Acid (E) | 68 | 5.7 |
| Histidine (H) | 12 | 1.0 |
| Lysine (K) | 24 | 2.0 |
| Methionine (M) | 7 | 0.59 |
| Asparagine (N) | 0 | 0 |
| Glutamine (Q) | 39 | 3.3 |
| Arginine (R) | 19 | 1.6 |
| Serine (S) | 5 | 0.42 |
| Threonine (T) | 6 | 0.50 |
| Tryptophan (W) | 3 | 0.25 |
| Tyrosine (Y) | 17 | 21.4 |

### Example 10

### Proteins Modified by Purine-Based Probes

Chemical proteomics performed as described in Example 9 determined several protein modification sites targeted by Pu-1 and/or Pu-2. Tables 3 and 4, below, lists sites of modification targeted by Pu-1 and Pu-2, respectively. The format of the tables is as follows: protein species, protein Uniprot accession number, cysteine sites (amino acid positions) modified (where if multiple sites are modified in the same protein, the sites are separated by vertical lines).

TABLE 3
Protein Modification Sites Targeted by Pu-1.

| Gene name | UniProt | Pu-1-modified Site |
|---|---|---|
| TACC3_HUMAN | Q9Y6A5 | 242\| |
| TNPO3_HUMAN | Q9Y5L0 | 511\| |
| CD2AP_HUMAN | Q9Y5K6 | 540\| |
| PPME1_HUMAN | Q9Y570 | 381\| |
| PRC2C_HUMAN | Q9Y520 | 895\| |
| FARP1_HUMAN | Q9Y4F1 | 522\| |
| WAC2C_HUMAN | Q9Y4E1 | 828\| |
| KDM3A_HUMAN | Q9Y4C1 | 1140\| |
| TLN1_HUMAN | Q9Y490 | 1939\| |
| RBGP1_HUMAN | Q9Y3P9 | 476\| |
| STRAP_HUMAN | Q9Y3F4 | 340\|305\| |
| UFC1_HUMAN | Q9Y3C8 | 116\| |
| SF3B6_HUMAN | Q9Y3B4 | 74\| |
| LC7L2_HUMAN | Q9Y383 | 348\| |
| AR2BP_HUMAN | Q9Y2Y0 | 149\| |
| 2ABG_HUMAN | Q9Y2T4 | 330\| |
| GUAD_HUMAN | Q9Y2T3 | 24\| |
| PDIP2_HUMAN | Q9Y2S7 | 143\| |
| RRP44_HUMAN | Q9Y2L1 | 533\| |
| ST38L_HUMAN | Q9Y2H1 | 235\| |
| AKAP2_HUMAN | Q9Y2D5 | 296\| |
| LZTS1_HUMAN | Q9Y250 | 385\|261\| |
| SRRM2_HUMAN | Q9UQ35 | 1029\| |
| UBP24_HUMAN | Q9UPU5 | 1556\| |
| PZRN3_HUMAN | Q9UPQ7 | 1002\| |
| LIMC1_HUMAN | Q9UPQ0 | 577\|333\|182\| |
| TRI33_HUMAN | Q9UPN9 | 943\|786 |
| TTF2_HUMAN | Q9UNY4 | 204\| |
| TIM_HUMAN | Q9UNS1 | 1126\| |
| PACN2_HUMAN | Q9UNF0 | 163\| |
| SOX13_HUMAN | Q9UN79 | 33\| |
| NOL7_HUMAN | Q9UMY1 | 149\| |
| TPX2_HUMAN | Q9ULW0 | 536\|301\| |
| COR1C_HUMAN | Q9ULV4 | 456\| |
| MRTFB_HUMAN | Q9ULH7 | 282\| |
| NUP50_HUMAN | Q9UKX7 | 333\| |
| ACINU_HUMAN | Q9UKV3 | 546\| |
| TF3C4_HUMAN | Q9UKN8 | 116\| |
| BAZ1B_HUMAN | Q9UIG0 | 338\| |
| BI2L1_HUMAN | Q9UHR4 | 182\| |
| NARF_HUMAN | Q9UHQ1 | 99\| |
| CHRD1_HUMAN | Q9UHD1 | 86\| |
| ARP21_HUMAN | Q9UBL0 | 321\| |
| ORC3_HUMAN | Q9UBD5 | 483\| |
| NCDN_HUMAN | Q9UBB6 | 98\| |
| RBM27_HUMAN | Q9P2N5 | 840\| |
| SYLC_HUMAN | Q9P2J5 | 554\| |
| RRBP1_HUMAN | Q9P2E9 | 892\|1216\| |
| BCCIP_HUMAN | Q9P287 | 213\| |
| UBP36_HUMAN | Q9P275 | 638\| |
| RCC2_HUMAN | Q9P258 | 280\| |
| K1522_HUMAN | Q9P206 | 491\| |
| F120A_HUMAN | Q9NZB2 | 531\| |
| TRM1_HUMAN | Q9NXH9 | 620\| |
| NHP2_HUMAN | Q9NX24 | 18\| |
| WDR70_HUMAN | Q9NW82 | 227\| |
| PNPO_HUMAN | Q9NVS9 | 156\| |
| TYDP1_HUMAN | Q9NUW8 | 135\| |
| KIF15_HUMAN | Q9NS87 | 576\| |
| STRN4_HUMAN | Q9NRL3 | 337\| |
| EI2BG_HUMAN | Q9NR50 | 281\| |
| SIAS_HUMAN | Q9NR45 | 283\| |
| DDX21_HUMAN | Q9NR30 | 378\| |
| ANLN_HUMAN | Q9NQW6 | 819\|512\|1117\| |
| AVEN_HUMAN | Q9NQS1 | 169\| |
| RRAGD_HUMAN | Q9NQL2 | 359\| |
| GPCP1_HUMAN | Q9NPB8 | 205\| |
| SYSM_HUMAN | Q9NP81 | 64\| |
| NCK5L_HUMAN | Q9HCH0 | 783\| |
| MCCB_HUMAN | Q9HCC0 | 267\| |
| SPC25_HUMAN | Q9HBM1 | 27\| |
| RRAGC_HUMAN | Q9HB90 | 377\|358\| |
| XPO5_HUMAN | Q9HAV4 | 941\|1157\| |
| PKHA5_HUMAN | Q9HAU0 | 891\| |
| CSN7B_HUMAN | Q9H9Q2 | 240\| |
| CNO10_HUMAN | Q9H9A5 | 504\| |
| DCTP1_HUMAN | Q9H773 | 162\| |
| RANB3_HUMAN | Q9H6Z4 | 249\|228\| |
| CCD86_HUMAN | Q9H6F5 | 116\| |
| RABE2_HUMAN | Q9H5N1 | 482\|115\| |
| WNK1_HUMAN | Q9H4A3 | 2292\| |
| NELFA_HUMAN | Q9H3P2 | 44\| |
| SLK_HUMAN | Q9H2G2 | 1153\| |
| TDIF1_HUMAN | Q9H147 | 156\| |
| CSTFT_HUMAN | Q9H0L4 | 222\| |
| XRN2_HUMAN | Q9H0D6 | 21\| |
| KLC2_HUMAN | Q9H0B6 | 474\| |
| PIP30_HUMAN | Q9GZU8 | 187\| |
| DDX24_HUMAN | Q9GZR7 | 832\| |
| PITH1_HUMAN | Q9GZP4 | 187\| |
| MTMRC_HUMAN | Q9C0I1 | 67\| |
| CEP44_HUMAN | Q9C0F1 | 28\| |
| UBE2O_HUMAN | Q9C0C9 | 341\| |
| TB182_HUMAN | Q9C0C2 | 794\|716\| |
| NIBA1_HUMAN | Q9BZQ8 | 516\| |
| TBD2A_HUMAN | Q9BYX2 | 686\| |
| FACD2_HUMAN | Q9BXW9 | 893\| |
| NAA15_HUMAN | Q9BXJ9 | 721\|214\| |
| SSBP3_HUMAN | Q9BWW4 | 80\| |
| NADAP_HUMAN | Q9BWU0 | 723\| |
| SSBP4_HUMAN | Q9BWG4 | 81\| |
| NUDT9_HUMAN | Q9BW91 | 347\| |
| GNL3_HUMAN | Q9BVP2 | 15\| |
| NUP58_HUMAN | Q9BVL2 | 252\| |
| MELPH_HUMAN | Q9BV36 | 277\| |
| DIDO1_HUMAN | Q9BTC0 | 350\| |
| NTPCR_HUMAN | Q9BSD7 | 101\| |
| CPPED_HUMAN | Q9BRF8 | 54\| |
| TBA1C_HUMAN | Q9BQE3 | 347\| |
| CND3_HUMAN | Q9BPX3 | 667\| |
| SH3G2_HUMAN | Q99962 | 108\| |
| SCAFB_HUMAN | Q99590 | 566\|1020\| |
| NUP88_HUMAN | Q99567 | 608\| |
| DNJC2_HUMAN | Q99543 | 240\| |
| VAT1_HUMAN | Q99536 | 86\| |
| PARK7_HUMAN | Q99497 | 46\| |
| PSMD1_HUMAN | Q99460 | 112\| |
| CNN2_HUMAN | Q99439 | 240\| |
| NIBA2_HUMAN | Q96TA1 | 466\| |
| RANB9_HUMAN | Q96S59 | 610\|594\| |
| NUDC1_HUMAN | Q96RS6 | 111\| |
| TGS1_HUMAN | Q96RS0 | 357\| |
| UIMC1_HUMAN | Q96RL1 | 257\|220\| |
| NACC1_HUMAN | Q96RE7 | 178\| |
| TRNT1_HUMAN | Q96Q11 | 373\| |
| DOCK7_HUMAN | Q96N67 | 2125\| |
| TRI47_HUMAN | Q96LD4 | 454\| |
| SNX27_HUMAN | Q96L92 | 30\| |
| SCYL1_HUMAN | Q96KG9 | 241\| |
| TOPK_HUMAN | Q96KB5 | 70\|22\| |
| UBP47_HUMAN | Q96K76 | 856\| |
| LRCH3_HUMAN | Q96II8 | 531\| |
| PDLI5_HUMAN | Q96HC4 | 213\| |
| ZCCHL_HUMAN | Q96H79 | 121\| |
| DTBP1_HUMAN | Q96EV8 | 302\| |
| DAZP1_HUMAN | Q96EP5 | 85\| |
| TCAL4_HUMAN | Q96EI5 | 34\| |
| ELP4_HUMAN | Q96EB1 | 218\| |
| NMD3_HUMAN | Q96D46 | 214\| |
| OPTN_HUMAN | Q96CV9 | 472\| |
| FWCH2_HUMAN | Q96CP2 | 132\| |
| EFHD2_HUMAN | Q96C19 | 53\| |
| NTAN1_HUMAN | Q96AB6 | 118\| |
| EXOC4_HUMAN | Q96A65 | 957\| |
| SYAP1_HUMAN | Q96A49 | 283\| |
| LPP_HUMAN | Q93052 | 364\|262\| |
| DDX17_HUMAN | Q92841 | 584\| |
| EZH1_HUMAN | Q92800 | 504\| |
| HS105_HUMAN | Q92598 | 845\| |
| PHF3_HUMAN | Q92576 | 402\| |
| TOPB1_HUMAN | Q92547 | 1166\| |
| DDB2_HUMAN | Q92466 | 364\|322\| |
| PALLD_HUMAN | Q8WX93 | 964\|429\| |
| LMO7_HUMAN | Q8WWI1 | 228\| |
| DNJA4_HUMAN | Q8WW22 | 368\| |
| LEO1_HUMAN | Q8WVC0 | 530\| |
| PDC6I_HUMAN | Q8WUM4 | 512\|40\| |
| GEMI5_HUMAN | Q8TEQ6 | 806\| |
| TBCK_HUMAN | Q8TEA7 | 737\| |
| DDX54_HUMAN | Q8TDD1 | 73\| |
| NEK9_HUMAN | Q8TD19 | 11\| |
| CIP2A_HUMAN | Q8TCG1 | 615\| |
| UBA3_HUMAN | Q8TBC4 | 28\| |
| THOC2_HUMAN | Q8NI27 | 1518\| |
| TTL_HUMAN | Q8NG68 | 238\| |
| BD1L1_HUMAN | Q8NFC6 | 686\|607\|2438\|1947\| |
| LS14A_HUMAN | Q8ND56 | 375\| |
| TXND5_HUMAN | Q8NBS9 | 350\| |
| UBR7_HUMAN | Q8N806 | 374\| |
| EH1L1_HUMAN | Q8N3D4 | 221\|1364 |
| CMTR1_HUMAN | Q8N1G2 | 9\| |
| NUP93_HUMAN | Q8N1F7 | 522\|422\| |
| CCAR2_HUMAN | Q8N163 | 644\| |
| SPART_HUMAN | Q8N0X7 | 405\| |
| PALM2_HUMAN | Q8IXS6 | 238\| |
| DHX40_HUMAN | Q8IX18 | 33\| |
| CHERP_HUMAN | Q8IWX8 | 69\| |
| GCC2_HUMAN | Q8IWJ2 | 416\| |
| MA7D3_HUMAN | Q8IWC1 | 494\| |
| AHNK2_HUMAN | Q8IVF2 | 5415\|225\|1872\| |
| ANKY2_HUMAN | Q8IV38 | 277\| |
| I2BP1_HUMAN | Q8IU81 | 363\| |
| DAAF5_HUMAN | Q86Y56 | 365\| |
| GA2L3_HUMAN | Q86XJ1 | 427\| |
| PRSR2_HUMAN | Q86WR7 | 367\| |
| NIPA_HUMAN | Q86WB0 | 208\| |
| MET16_HUMAN | Q86W50 | 448\|432\| |
| NOP9_HUMAN | Q86U38 | 242\| |
| HUWE1_HUMAN | Q7Z6Z7 | 790\|3658\|2721\|1628\|1421\|1401\| |
| RHG30_HUMAN | Q7Z6I6 | 654\| |
| I2BP2_HUMAN | Q7Z5L9 | 65\| |
| WAPL_HUMAN | Q7Z5K2 | 160\| |
| RAI1_HUMAN | Q7Z5J4 | 594\| |
| HDGR2_HUMAN | Q7Z4V5 | 631\| |
| HAUS6_HUMAN | Q7Z4H7 | 743\| |
| NUFP2_HUMAN | Q7Z417 | 234\| |
| MYH14_HUMAN | Q7Z406 | 954\| |
| ZFY16_HUMAN | Q7Z3T8 | 863\|304\| |
| SETX_HUMAN | Q7Z333 | 637\| |
| ZCCHV_HUMAN | Q7Z2W4 | 645\|272\| |
| TRM1L_HUMAN | Q7Z2T5 | 239\| |
| RSRC2_HUMAN | Q7L4I2 | 382\| |
| MEPCE_HUMAN | Q7L2J0 | 153\| |
| BZW1_HUMAN | Q7L1Q6 | 35\| |
| SND1_HUMAN | Q7KZF4 | 152\| |
| TBA1A_HUMAN | Q71U36 | 347\| |
| DUSTY_HUMAN | Q6XUX3 | 57\| |
| CPLX2_HUMAN | Q6PUV4 | 90\| |
| LARP1_HUMAN | Q6PKG0 | 238\| |
| PRP8_HUMAN | Q6P2Q9 | 435\| |
| CDC73_HUMAN | Q6P1J9 | 145\| |
| ZCHC8_HUMAN | Q6NZY4 | 607\| |
| PPR18_HUMAN | Q6NYC8 | 276\| |
| TTI2_HUMAN | Q6NXR4 | 36\| |
| IF5AL_HUMAN | Q6IS14 | 73\| |
| TWF2_HUMAN | Q6IBS0 | 141\| |
| CPIN1_HUMAN | Q6FI81 | 274\|237\| |
| TENS3_HUMAN | Q68CZ2 | 1241\| |
| KANK2_HUMAN | Q63ZY3 | 389\| |
| RPRD2_HUMAN | Q5VT52 | 903\| |
| LYRM7_HUMAN | Q5U5X0 | 97\| |
| UBAP2_HUMAN | Q5T6F2 | 208\| |
| UBR4_HUMAN | Q5T4S7 | 3430\| |
| RRP12_HUMAN | Q5JTH9 | 31\| |
| PRC2B_HUMAN | Q5JSZ5 | 1184\|1121\| |
| PP6R3_HUMAN | Q5H9R7 | 844\|830\| |
| SGO1_HUMAN | Q5FBB7 | 503\| |
| RIPL1_HUMAN | Q5EBL4 | 47\| |
| RHG15_HUMAN | Q53QZ3 | 140\| |
| LRRF1_HUMAN | Q32MZ4 | 726\|644\|14\| |
| TSR1_HUMAN | Q2NL82 | 126\| |
| ERC6L_HUMAN | Q2NKX8 | 825\| |
| PDS5A_HUMAN | Q29RF7 | 532\| |
| INF2_HUMAN | Q27J81 | 971\|1029\| |
| UPP1_HUMAN | Q16831 | 89\|225\|17\| |
| KYNU_HUMAN | Q16719 | 45\| |
| MAPK3_HUMAN | Q16644 | 379\| |
| DREB_HUMAN | Q16643 | 613\| |
| RBBP7_HUMAN | Q16576 | 97\| |
| DPYL2_HUMAN | Q16555 | 504\| |
| SNPC1_HUMAN | Q16533 | 256\| |
| ADRM1_HUMAN | Q16186 | 121\| |
| EZH2_HUMAN | Q15910 | 503\| |
| NAB2_HUMAN | Q15742 | 499\| |
| PCH2_HUMAN | Q15645 | 14\| |
| TSN_HUMAN | Q15631 | 225\| |
| SURF2_HUMAN | Q15527 | 127\| |
| SKIV2_HUMAN | Q15477 | 913\| |
| KS6A1_HUMAN | Q15418 | 432\|223\| |
| CNN3_HUMAN | Q15417 | 173\| |
| PCBP2_HUMAN | Q15366 | 217\| |
| PCBP1_HUMAN | Q15365 | 54\|355\|158\| |
| TEBP_HUMAN | Q15185 | 58\| |
| IPYR_HUMAN | Q15181 | 270\| |
| PLEC_HUMAN | Q15149 | 4494\| |
| KIF14_HUMAN | Q15058 | 1640\| |
| FL2D_HUMAN | Q15007 | 270\| |
| CND2_HUMAN | Q15003 | 418\|255\|114\| |
| GAPD1_HUMAN | Q14C86 | 568\| |
| NUMA1_HUMAN | Q14980 | 961\|1937\|1907\| |
| CHD4_HUMAN | Q14839 | 1594\| |
| GOGB1_HUMAN | Q14789 | 681\| |
| UBP10_HUMAN | Q14694 | 254\| |
| LAGE3_HUMAN | Q14657 | 23\| |
| ZN460_HUMAN | Q14592 | 181\| |
| PDE3A_HUMAN | Q14432 | 526\|407\| |
| SRC8_HUMAN | Q14247 | 246\|112\| |
| ELOA1_HUMAN | Q14241 | 339\| |
| NPAT_HUMAN | Q14207 | 1172\| |
| DYHC1_HUMAN | Q14204 | 633\|4121\| |
| DCTN1_HUMAN | Q14203 | 1252\| |
| DPOA2_HUMAN | Q14181 | 82\| |
| MORC3_HUMAN | Q14149 | 694\|15\| |
| DSG2_HUMAN | Q14126 | 871\| |
| CKAP5_HUMAN | Q14008 | 1795\|1113\| |
| CUL4B_HUMAN | Q13620 | 787\| |
| CUL4A_HUMAN | Q13619 | 633\|241\| |
| CUL3_HUMAN | Q13618 | 636\| |
| HDAC1_HUMAN | Q13547 | 408\| |
| SQSTM_HUMAN | Q13501 | 331\|113\| |
| GOGA4_HUMAN | Q13439 | 853\|1729\| |
| SNTB2_HUMAN | Q13425 | 391\| |
| ATM_HUMAN | Q13315 | 564\| |
| STK3_HUMAN | Q13188 | 410\| |
| PRDX4_HUMAN | Q13162 | 51\| |
| AIMP2_HUMAN | Q13155 | 306\| |
| AAPK1_HUMAN | Q13131 | 425\| |
| TRAF2_HUMAN | Q12933 | 124\| |
| TRAP1_HUMAN | Q12931 | 573\| |
| TP53B_HUMAN | Q12888 | 896\|319\|1703\|1280\| |
| CHD3_HUMAN | Q12873 | 1838\| |
| AKP13_HUMAN | Q12802 | 1666\| |
| NU160_HUMAN | Q12769 | 1166\| |
| AHNK_HUMAN | Q09666 | 5502\|2806\|1967\|1900\| |
| NCBP1_HUMAN | Q09161 | 503\| |
| NSUN2_HUMAN | Q08J23 | 758\|599\|502\| |
| PRDX1_HUMAN | Q06830 | 71\|173\| |
| PSME1_HUMAN | Q06323 | 101\| |
| GFPT1_HUMAN | Q06210 | 264\| |
| CALD1_HUMAN | Q05682 | 636\| |
| LGUL_HUMAN | Q04760 | 139\| |
| K1C17_HUMAN | Q04695 | 40\|358\| |
| IF4G1_HUMAN | Q04637 | 662\| |
| GLGB_HUMAN | Q04446 | 81\|221\| |
| DYST_HUMAN | Q03001 | 3168\| |
| AKA12_HUMAN | Q02952 | 578\|1521\|1314\| |
| KIF23_HUMAN | Q02241 | 629\| |
| AMPD2_HUMAN | Q01433 | 123\| |
| HNRPU_HUMAN | Q00839 | 594\| |
| CDK17_HUMAN | Q00537 | 233\| |
| CDK16_HUMAN | Q00536 | 206\| |
| CDK6_HUMAN | Q00534 | 15\| |
| VIGLN_HUMAN | Q00341 | 940\| |
| 2ABB_HUMAN | Q00005 | 330\| |
| H33_HUMAN | P84243 | 111\| |
| SRSF3_HUMAN | P84103 | 6\| |
| COG7_HUMAN | P83436 | 505\| |
| PRKDC_HUMAN | P78527 | 3837\| |
| GSTO1_HUMAN | P78417 | 192\| |
| TCPB_HUMAN | P78371 | 535\| |
| GTF2I_HUMAN | P78347 | 215\| |
| RPP30_HUMAN | P78346 | 257\| |
| TBB4B_HUMAN | P68371 | 303\|12\| |
| TBA4A_HUMAN | P68366 | 54\| |
| TBA1B_HUMAN | P68363 | 347\|315\|295\| |
| PP2AA_HUMAN | P67775 | 266\|251\| |
| ACTG_HUMAN | P63261 | 285\|257\| |
| IF5A1_HUMAN | P63241 | 73\| |
| SKP1_HUMAN | P63208 | 160\| |
| 2ABA_HUMAN | P63151 | 334\| |
| 1433Z_HUMAN | P63104 | 25\| |
| RAC1_HUMAN | P63000 | 178\|105\| |
| GRB2_HUMAN | P62993 | 32\| |
| HNRPK_HUMAN | P61978 | 184\|145\|132\| |
| PSME3_HUMAN | P61289 | 92\| |
| DEST_HUMAN | P60981 | 39\|23\|135\| |
| RAC3_HUMAN | P60763 | 178\| |
| GSDMD_HUMAN | P57764 | 268\| |
| NU107_HUMAN | P57740 | 78\| |
| WDR4_HUMAN | P57081 | 412\| |
| SOX10_HUMAN | P56693 | 22\| |
| HNRH2_HUMAN | P55795 | 267\| |
| PSA_HUMAN | P55786 | 265\| |
| DSRAD_HUMAN | P55265 | 773\| |
| CASP6_HUMAN | P55212 | 163\| |
| NP1L1_HUMAN | P55209 | 132\| |
| ELL_HUMAN | P55199 | 411\| |
| PMS2_HUMAN | P54278 | 591\| |
| SYRC_HUMAN | P54136 | 32\| |
| ICLN_HUMAN | P54105 | 73\| |
| COPB_HUMAN | P53618 | 684\|623\| |
| ACLY_HUMAN | P53396 | 20\| |
| PLK1_HUMAN | P53350 | 372\| |
| BIEA_HUMAN | P53004 | 204\| |
| AK1C2_HUMAN | P52895 | 7\|206\|188\| |
| THOP1_HUMAN | P52888 | 350\| |
| MSH6_HUMAN | P52701 | 615\| |
| HNRPF_HUMAN | P52597 | 267\| |
| 6PGD_HUMAN | P52209 | 402\|170\| |
| KS6A3_HUMAN | P51812 | 229\| |
| MECP2_HUMAN | P51608 | 429\| |
| LRBA_HUMAN | P50851 | 1228\| |
| DYN2_HUMAN | P50570 | 86\| |
| VASP_HUMAN | P50552 | 334\| |
| ERF_HUMAN | P50548 | 477\| |
| SPB9_HUMAN | P50453 | 259\| |
| GUAA_HUMAN | P49915 | 523\|449\| |
| RBP2_HUMAN | P49792 | 3071\|2696\|220\| |
| NU153_HUMAN | P49790 | 593\|21\|1129\| |
| SYAC_HUMAN | P49588 | 773\| |
| SRP09_HUMAN | P49458 | 39\| |
| FAS_HUMAN | P49327 | 779\|2202\|1548\| |
| NASP_HUMAN | P49321 | 254\| |
| NEST_HUMAN | P48681 | 575\| |
| TCPE_HUMAN | P48643 | 407\| |
| PPCE_HUMAN | P48147 | 255\| |
| CAPZB_HUMAN | P47756 | 206\| |
| UTRO_HUMAN | P46939 | 277\| |
| MAP1B_HUMAN | P46821 | 2065\|2041\|1228\| |
| ATRX_HUMAN | P46100 | 2404\| |
| RECQ1_HUMAN | P46063 | 606\|49\| |
| KI67_HUMAN | P46013 | 226\|1479\| |
| CBX5_HUMAN | P45973 | 133\| |
| RANG_HUMAN | P43487 | 158\| |
| MAGA3_HUMAN | P43357 | 10\| |
| MSH2_HUMAN | P43246 | 873\| |
| CASP2_HUMAN | P42575 | 343\| |
| MTOR_HUMAN | P42345 | 300\| |
| STAT1_HUMAN | P42224 | 492\| |
| LAP2A_HUMAN | P42166 | 684\|287\| |
| GARS_HUMAN | P41250 | 616\|466\| |
| CSK_HUMAN | P41240 | 31\| |
| NAA10_HUMAN | P41227 | 194\| |
| IF2G_HUMAN | P41091 | 105\| |
| UBP8_HUMAN | P40818 | 809\| |
| STAT3_HUMAN | P40763 | 687\| |
| TAGL2_HUMAN | P37802 | 124\| |
| MYH9_HUMAN | P35579 | 917\|172\|1379\| |
| SPB6_HUMAN | P35237 | 350\| |
| CTNA1_HUMAN | P35221 | 116\| |
| HSP74_HUMAN | P34932 | 270\| |
| MCM5_HUMAN | P33992 | 197\| |
| CSTF2_HUMAN | P33240 | 150\| |
| PRDX2_HUMAN | P32119 | 172\| |
| 1433S_HUMAN | P31947 | 38\| |
| HNRH1_HUMAN | P31943 | 267\| |
| CPSM_HUMAN | P31327 | 816\| |
| PRDX6_HUMAN | P30041 | 91\| |
| PML_HUMAN | P29590 | 389\| |
| TKT_HUMAN | P29401 | 41\|133\| |
| TPP2_HUMAN | P29144 | 28\| |
| ERCC5_HUMAN | P28715 | 550\| |
| MAP4_HUMAN | P27816 | 1098\| |
| PYR1_HUMAN | P27708 | 736\|1889\| |
| DCK_HUMAN | P27707 | 59\|45\| |
| ARNT_HUMAN | P27540 | 62\| |
| EF1G_HUMAN | P26641 | 266\|210\| |
| DNMT1_HUMAN | P26358 | 62\|41\| |
| DDX6_HUMAN | P26196 | 102\| |
| RS12_HUMAN | P25398 | 130\| |
| KTHY_HUMAN | P23919 | 31\| |
| IF4B_HUMAN | P23588 | 457\| |
| COF1_HUMAN | P23528 | 147\|139\| |
| SYWC_HUMAN | P23381 | 62\| |
| PUR6_HUMAN | P22234 | 63\| |
| OSBP1_HUMAN | P22059 | 343\| |
| TGM2_HUMAN | P21980 | 545\|230\| |
| FLNA_HUMAN | P21333 | 733\|717\|623\|53\|478\|2582\|2543\|205\|1353\|1157\| |
| CD11B_HUMAN | P21127 | 440\| |
| ICAL_HUMAN | P20810 | 661\|328\|241\| |
| ANXA7_HUMAN | P20073 | 363\| |
| NFKB1_HUMAN | P19838 | 61\| |
| CSK22_HUMAN | P19784 | 336\| |
| NUCL_HUMAN | P19338 | 543\| |
| PYRG1_HUMAN | P17812 | 491\|362\| |
| CAN2_HUMAN | P17655 | 82\|374\| |
| ZNF24_HUMAN | P17028 | 189\| |
| RS2_HUMAN | P15880 | 182\| |
| ERF3A_HUMAN | P15170 | 327\| |
| PLAK_HUMAN | P14923 | 49\| |
| HNRPL_HUMAN | P14866 | 472\| |
| MIF_HUMAN | P14174 | 81\| |
| PLST_HUMAN | P13797 | 104\| |
| PLSL_HUMAN | P13796 | 101\| |
| TCTP_HUMAN | P13693 | 172\| |
| EF2_HUMAN | P13639 | 67\|651\|41\|369\| |
| ACTN1_HUMAN | P12814 | 370\|154\| |
| IMDH2_HUMAN | P12268 | 173\| |
| PABP1_HUMAN | P11940 | 132\| |
| G6PD_HUMAN | P11413 | 446\|385\|158\|13\| |
| PYGB_HUMAN | P11216 | 437\|319\| |
| KAP0_HUMAN | P10644 | 18\| |
| THIO_HUMAN | P10599 | 73\|32\| |
| ARAF_HUMAN | P10398 | 192\| |
| CTF8_HUMAN | P0CG13 | 83\| |
| UCHL1_HUMAN | P09936 | 90\| |
| ROA1_HUMAN | P09651 | 175\| |
| HMGB1_HUMAN | P09429 | 23\|106\| |
| HS90B_HUMAN | P08238 | 589\|564\| |
| SYEP_HUMAN | P07814 | 856\|1480\| |
| TBB5_HUMAN | P07437 | 303\|12\| |
| LDHB_HUMAN | P07195 | 36\| |
| GELS_HUMAN | P06396 | 331\| |
| P53_HUMAN | P04637 | 141\| |
| KITH_HUMAN | P04183 | 206\| |
| GCR_HUMAN | P04150 | 287\| |
| ALDOA_HUMAN | P04075 | 178\| |
| PNPH_HUMAN | P00491 | 31\| |
| AL1A1_HUMAN | P00352 | 50\|370\|186\|133\|126\| |
| LDHA_HUMAN | P00338 | 163\| |
| NSD2_HUMAN | O96028 | 406\| |
| BAG3_HUMAN | O95817 | 179\|151\| |
| DDX58_HUMAN | O95786 | 490\| |
| HS74L_HUMAN | O95757 | 540\|417\| |
| IPO7_HUMAN | O95373 | 90\|757\| |
| TRI16_HUMAN | O95361 | 178\| |
| KIF4A_HUMAN | O95239 | 1224\|1171\| |
| ELP1_HUMAN | O95163 | 453\| |
| UBXN7_HUMAN | O94888 | 160\| |
| STK10_HUMAN | O94804 | 947\|888\| |
| WDHD1_HUMAN | O75717 | 773\| |
| NU155_HUMAN | O75694 | 1344\| |
| SURF6_HUMAN | O75683 | 189\| |
| TIPRL_HUMAN | O75663 | 87\| |
| CSDE1_HUMAN | O75534 | 680\| |
| NCOR1_HUMAN | O75376 | 2322\| |
| FLNB_HUMAN | O75369 | 1868\|1326\| |
| VATG1_HUMAN | O75348 | 69\| |
| BRE1B_HUMAN | O75150 | 890\| |
| ROCK2_HUMAN | O75116 | 649\|1257\| |
| IF2P_HUMAN | O60841 | 1158\| |
| DIAP1_HUMAN | O60610 | 1227\| |
| TBCD4_HUMAN | O60343 | 74\| |
| GANP_HUMAN | O60318 | 1864\| |
| MYO1B_HUMAN | O43795 | 613\| |
| SGTA_HUMAN | O43765 | 148\| |
| HTSF1_HUMAN | O43719 | 512\|480\|462\| |
| ACTN4_HUMAN | O43707 | 173\| |
| XPOT_HUMAN | O43592 | 650\| |
| WIPF1_HUMAN | O43516 | 446\| |
| IF4G3_HUMAN | O43432 | 1411\| |
| HNRPR_HUMAN | O43390 | 226\| |
| SNUT1_HUMAN | O43290 | 645\| |
| SERA_HUMAN | O43175 | 281\|18\| |
| MCES_HUMAN | O43148 | 95\| |
| EIF3D_HUMAN | O15371 | 19\| |
| PPM1G_HUMAN | O15355 | 241\| |
| IKKA_HUMAN | O15111 | 406\| |
| PUR4_HUMAN | O15067 | 606\| |
| ARHGA_HUMAN | O15013 | 401\| |
| XPO1_HUMAN | O14980 | 34\|1070\| |
| CSKP_HUMAN | O14936 | 418\| |
| HAT1_HUMAN | O14929 | 101\| |
| GEMI2_HUMAN | O14893 | 63\| |
| IRS4_HUMAN | O14654 | 522\| |
| PDXK_HUMAN | O00764 | 273\| |
| EXOC5_HUMAN | O00471 | 194\| |
| DNM1L_HUMAN | O00429 | 470\| |
| IPO5_HUMAN | O00410 | 915\| |
| CLIC1_HUMAN | O00299 | 59\| |
| SPT5H_HUMAN | O00267 | 740\| |
| PSMD9_HUMAN | O00233 | 81\|59\| |
| SBNO1_HUMAN | A3KN83 | 445\| |
| SPD2B_HUMAN | A1X283 | 598\| |

TABLE 4
Protein Modification Sites Targeted by Pu-2.

| Gene name | UniProt | Pu-2-modified Site |
|---|---|---|
| UBA6_HUMAN | A0AVT1 | 347\| |
| MED19_HUMAN | A0JLT2 | 62\| |
| SPD2B_HUMAN | A1X283 | 783\|598\| |
| SBNO1_HUMAN | A3KN83 | 445\|699\| |
| WDR91_HUMAN | A4D1P6 | 351\|366\| |
| SMHD1_HUMAN | A6NHR9 | 1656\|897\| |
| PSD11_HUMAN | O00231 | 289\| |
| DFFA_HUMAN | O00273 | 165\|38\| |
| WASL_HUMAN | O00401 | 431\| |
| ARI1A_HUMAN | O14497 | 1874\| |
| UB2L6_HUMAN | O14933 | 86\| |
| AURKA_HUMAN | O14965 | 49\| |
| BTAF1_HUMAN | O14981 | 109\| |
| SPTN2_HUMAN | O15020 | 231\| |
| PUR4_HUMAN | O15067 | 1285\|606\|66\|1044\| |
| INP4_BHUMAN | O15327 | 534\| |
| PPM1G_HUMAN | O15355 | 241\|164\| |
| R113A_HUMAN | O15541 | 15\| |
| MCES_HUMAN | O43148 | 73\|95\| |
| SERA_HUMAN | O43175 | 369\|18\| |
| DC1L2_HUMAN | O43237 | 191\| |
| ERI3_HUMAN | O43414 | 285\| |
| IF4G3_HUMAN | O43432 | 1411\| |
| XPOT_HUMAN | O43592 | 650\| |
| HTSF1_HUMAN | O43719 | 512\|462\| |
| STRN_HUMAN | O43815 | 765\| |
| TBCD4_HUMAN | O60343 | 1286\|316\|45\|1277\| |
| GSDME_HUMAN | O60443 | 180\| |
| OGA_HUMAN | O60502 | 596\| |
| HNRPQ_HUMAN | O60506 | 96\| |
| USO1_HUMAN | O60763 | 678\| |
| KIN17_HUMAN | O60870 | 212\| |
| NBN_HUMAN | O60934 | 487\| |
| SRGP2_HUMAN | O75044 | 820\|357\| |
| N4BP1_HUMAN | O75113 | 483\| |
| PP6R2_HUMAN | O75170 | 880\|761\|870\| |
| MPPB_HUMAN | O75439 | 265\| |
| KS6A5_HUMAN | O75582 | 214\| |
| TIPRL_HUMAN | O75663 | 14\|87\| |
| GLRX3_HUMAN | O76003 | 146\| |
| CIAO1_HUMAN | O76071 | 234\| |
| STK10_HUMAN | O94804 | 888\| |
| PLPHP_HUMAN | O94903 | 261\| |
| HEXI1_HUMAN | O94992 | 79\| |
| UBR5_HUMAN | O95071 | 2267\| |
| UBE4B_HUMAN | O95155 | 113\| |
| ATE1_HUMAN | O95260 | 138\| |
| 6PGL_HUMAN | O95336 | 32\| |
| SMC2_HUMAN | O95347 | 1174\| |
| SVIL_HUMAN | O95425 | 26\|671\| |
| BAG3_HUMAN | O95817 | 179\|151\| |
| LMNA_HUMAN | P02545 | 588\| |
| ALDOA_HUMAN | P04075 | 290\| |
| KITH_HUMAN | P04183 | 206\| |
| P53_HUMAN | P04637 | 135\|182\|141\| |
| TBB5_HUMAN | P07437 | 12\|127\|303\| |
| SYEP_HUMAN | P07814 | 744\|856\|1377\| |
| ARAF_HUMAN | P10398 | 597\|192\| |
| THIO_HUMAN | P10599 | 32\|73\| |
| G6PD_HUMAN | P11413 | 13\| |
| ACTN1_HUMAN | P12814 | 370\| |
| PEPD_HUMAN | P12955 | 467\|478\| |
| RINI_HUMAN | P13489 | 409\| |
| EF2_HUMAN | P13639 | 41\| |
| FAAA_HUMAN | P16930 | 105\| |
| ZNF24_HUMAN | P17028 | 233\| |
| SON_HUMAN | P18583 | 2070\| |
| NFKB1_HUMAN | P19838 | 61\| |
| VATC1_HUMAN | P21283 | 15\| |
| FLNA_HUMAN | P21333 | 1157\|1260\|2543\|574\|717\| |
| NF1_HUMAN | P21359 | 1032\|454\| |
| OSBP1_HUMAN | P22059 | 343\| |
| PUR2_HUMAN | P22102 | 41\| |
| SP100_HUMAN | P23497 | 238\| |
| COF1_HUMAN | P23528 | 147\|139\| |
| IF4B_HUMAN | P23588 | 457\| |
| CDK2_HUMAN | P24941 | 177\| |
| SYVC_HUMAN | P26640 | 41\| |
| MAP4_HUMAN | P27816 | 1008\|635\|535\|1098\| |
| IP3KB_HUMAN | P27987 | 379\| |
| RXRB_HUMAN | P28702 | 191\| |
| PML_HUMAN | P29590 | 338\|389\| |
| TF2H1_HUMAN | P32780 | 181\| |
| CGL_HUMAN | P32929 | 229\| |
| HSP74_HUMAN | P34932 | 34\|417\| |
| CAH8_HUMAN | P35219 | 200\|266\| |
| MYH9_HUMAN | P35579 | 988\|1437\|569\|1379\| |
| MYH10_HUMAN | P35580 | 1238\| |
| ADDA_HUMAN | P35611 | 430\|525\| |
| NU214_HUMAN | P35658 | 1003\| |
| TAGL2_HUMAN | P37802 | 124\| |
| NAA10_HUMAN | P41227 | 194\| |
| GARS_HUMAN | P41250 | 466\| |
| LAP2A_HUMAN | P42166 | 287\|280\|658\|330\| |
| EPS15_HUMAN | P42566 | 657\| |
| RANG_HUMAN | P43487 | 132\|99\| |
| CBX5_HUMAN | P45973 | 133\| |
| KI67_HUMAN | P46013 | 1843\|226\| |
| CRKL_HUMAN | P46109 | 249\| |
| MP2K3_HUMAN | P46734 | 227\| |
| MAP1B_HUMAN | P46821 | 2041\|2065\|905\|1814\|2083\|1228\| |
| UTRO_HUMAN | P46939 | 277\| |
| CAPZB_HUMAN | P47756 | 206\|147\| |
| NEST_HUMAN | P48681 | 157\|575\|161\| |
| NASP_HUMAN | P49321 | 708\|254\| |
| FAS_HUMAN | P49327 | 1448\|2202\| |
| NU153_HUMAN | P49790 | 1065\|404\|585\|1129\| |
| RBP2_HUMAN | P49792 | 3032\| |
| GUAA_HUMAN | P49915 | 631\| |
| PAPOA_HUMAN | P51003 | 36\| |
| HCFC1_HUMAN | P51610 | 1886\|227\| |
| DYLT3_HUMAN | P51808 | 8\| |
| HDGF_HUMAN | P51858 | 12\|108\| |
| HNRPM_HUMAN | P52272 | 653\| |
| HNRPF_HUMAN | P52597 | 267\| |
| STAT2_HUMAN | P52630 | 174\| |
| NUBP1_HUMAN | P53384 | 31\| |
| SC24C_HUMAN | P53992 | 78\| |
| ICLN_HUMAN | P54105 | 73\| |
| PMS2_HUMAN | P54278 | 216\|591\| |
| HSP72_HUMAN | P54652 | 191\| |
| XPO2_HUMAN | P55060 | 939\| |
| SYMC_HUMAN | P56192 | 441\| |
| GSDMD_HUMAN | P57764 | 191\| |
| DEST_HUMAN | P60981 | 135\|147\|23\| |
| HNRPK_HUMAN | P61978 | 184\| |
| PPIA_HUMAN | P62937 | 62\| |
| RAC1_HUMAN | P63000 | 178\| |
| DYL1_HUMAN | P63167 | 24\| |
| IF5A1_HUMAN | P63241 | 73\| |
| TBA1B_HUMAN | P68363 | 315\|347\| |
| TBB4B_HUMAN | P68371 | 12\|303\| |
| HSF1_HUMAN | Q00613 | 153\| |
| HNRPU_HUMAN | Q00839 | 594\| |
| CAP1_HUMAN | Q01518 | 93\| |
| RL18A_HUMAN | Q02543 | 22\| |
| TF65_HUMAN | Q04206 | 109\|105\| |
| GLGB_HUMAN | Q04446 | 221\|81\| |
| IF4G1_HUMAN | Q04637 | 662\| |
| PTN12_HUMAN | Q05209 | 495\| |
| PTN11_HUMAN | Q06124 | 259\| |
| PSME1_HUMAN | Q06323 | 101\|22\| |
| PRDX1_HUMAN | Q06830 | 173\| |
| KLC1_HUMAN | Q07866 | 114\| |
| GOGA3_HUMAN | Q08378 | 45\|455\| |
| CAMP2_HUMAN | Q08AD1 | 675\| |
| SLFN5_HUMAN | Q08AF3 | 362\| |
| VAC14_HUMAN | Q08AM6 | 516\| |
| NCBP1_HUMAN | Q09161 | 36\|44\| |
| AHNK_HUMAN | Q09666 | 1900\|2806\|1833\| |
| GRSF1_HUMAN | Q12849 | 476\| |
| TP53B_HUMAN | Q12888 | 1038\|1159\|319\|101\|772\|1375\| |
| TRAF2_HUMAN | Q12933 | 287\| |
| CDC16_HUMAN | Q13042 | 544\| |
| ATM_HUMAN | Q13315 | 219\|2323\|2607\|2991\|564\|384\|1396\| |
| ILK_HUMAN | Q13418 | 346\|422\| |
| GOGA4_HUMAN | Q13439 | 1729\|1340\|1771\| |
| MYO9B_HUMAN | Q13459 | 1169\| |
| SQSTM_HUMAN | Q13501 | 331\|289\|113\| |
| ITPK1_HUMAN | Q13572 | 391\| |
| RIN1_HUMAN | Q13671 | 733\| |
| SHRM2_HUMAN | Q13796 | 1231\| |
| MORC3_HUMAN | Q14149 | 619\|671\|694\| |
| SAFB2_HUMAN | Q14151 | 361\| |
| SRC8_HUMAN | Q14247 | 112\|246\| |
| HLTF_HUMAN | Q14527 | 442\|461\| |
| LAGE3_HUMAN | Q14657 | 23\| |
| SMC1A_HUMAN | Q14683 | 987\| |
| UBP10_HUMAN | Q14694 | 94\|254\| |
| GOGB1_HUMAN | Q14789 | 1462\|3070\|3144\|1811\| |
| MEF2D_HUMAN | Q14814 | 217\| |
| NUMA1_HUMAN | Q14980 | 1136\|1729\|1907\|2009\|1937\|961\| |
| GAPD1_HUMAN | Q14C86 | 293\|568\|741\| |
| SPCS2_HUMAN | Q15005 | 17\| |
| SART3_HUMAN | Q15020 | 537\|670\| |
| IPYR_HUMAN | Q15181 | 270\| |
| TEBP_HUMAN | Q15185 | 40\|75\|58\| |
| PCBP1_HUMAN | Q15365 | 109\|54\|163\| |
| PCBP2_HUMAN | Q15366 | 109\| |
| CNN3_HUMAN | Q15417 | 273\|173\| |
| SAFB1_HUMAN | Q15424 | 362\| |
| SKIV2_HUMAN | Q15477 | 247\| |
| SURF2_HUMAN | Q15527 | 116\|127\| |
| TSN_HUMAN | Q15631 | 225\| |
| TRIPB_HUMAN | Q15643 | 212\| |
| CDC37_HUMAN | Q16543 | 64\| |
| SMN_HUMAN | Q16637 | 60\| |
| DREB_HUMAN | Q16643 | 96\|613\| |
| MAR1_HUMAN | Q16655 | 68\| |
| IF16_HUMAN | Q16666 | 191\|637\| |
| UPP1_HUMAN | Q16831 | 17\|71\|162\| |
| PDS5A_HUMAN | Q29RF7 | 532\|1093\| |
| FA98B_HUMAN | Q52LJ0 | 216\| |
| RIPL1_HUMAN | Q5EBL4 | 47\| |
| ATPF1_HUMAN | Q5TC12 | 321\| |
| NT5D1_HUMAN | Q5TFE4 | 119\| |
| PR38B_HUMAN | Q5VTL8 | 113\| |
| MYOME_HUMAN | Q5VU43 | 603\| |
| LYPL1_HUMAN | Q5VWZ2 | 12\| |
| DEN4C_HUMAN | Q5VZ89 | 1113\| |
| RN213_HUMAN | Q63HN8 | 310\| |
| TENS3_HUMAN | Q68CZ2 | 928\|615\| |
| FMN1_HUMAN | Q68DA7 | 228\|660\| |
| ZFY26_HUMAN | Q68DK2 | 715\| |
| CPIN1_HUMAN | Q6FI81 | 237\|249\| |
| OTU7B_HUMAN | Q6GQQ9 | 708\| |
| TWF2_HUMAN | Q6IBS0 | 141\| |
| IF5AL_HUMAN | Q6IS14 | 73\| |
| PPR18_HUMAN | Q6NYC8 | 611\| |
| EDC4_HUMAN | Q6P2E9 | 54\|838\| |
| ANM9_HUMAN | Q6P2P2 | 290\| |
| ZC3HE_HUMAN | Q6PJT7 | 261\| |
| LARP1_HUMAN | Q6PKG0 | 238\| |
| LRSM1_HUMAN | Q6UWE0 | 23\| |
| DNMBP_HUMAN | Q6XZF7 | 691\| |
| YJ005_HUMAN | Q6ZSR9 | 251\| |
| RAPH1_HUMAN | Q70E73 | 847\| |
| EIF3M_HUMAN | Q7L2H7 | 175\| |
| MEPCE_HUMAN | Q7L2J0 | 429\|54\| |
| CYFP1_HUMAN | Q7L576 | 98\| |
| ZFY16_HUMAN | Q7Z3T8 | 1002\|122\|269\|304\|313\|377\|41\|62\|829\|863\|174\| |
| HDGR2_HUMAN | Q7Z4V5 | 631\| |
| WAPL_HUMAN | Q7Z5K2 | 160\|365\|906\| |
| PXK_HUMAN | Q7Z7A4 | 196\|570\| |
| CK096_HUMAN | Q7Z7L8 | 402\| |
| PRUN1_HUMAN | Q86TP1 | 437\| |
| MTA70_HUMAN | Q86U44 | 336\| |
| UBP48_HUMAN | Q86UV5 | 409\|986\|850\| |
| MET16_HUMAN | Q86W50 | 448\|480\|432\| |
| CACL1_HUMAN | Q86Y37 | 94\| |
| RB6I2_HUMAN | Q8IUD2 | 258\| |
| RHG12_HUMAN | Q8IWW6 | 199\| |
| ANKH1_HUMAN | Q8IWZ3 | 615\|873\| |
| SUGP1_HUMAN | Q8IWZ8 | 78\| |
| DHX40_HUMAN | Q8IX18 | 33\| |
| SPART_HUMAN | Q8N0X7 | 499\| |
| CMTR1_HUMAN | Q8N1G2 | 9\| |
| VPS8_HUMAN | Q8N3P4 | 974\| |
| OTU6B_HUMAN | Q8N6M0 | 172\| |
| CB069_HUMAN | Q8N8R5 | 230\| |
| NHLC2_HUMAN | Q8NBF2 | 716\| |
| MTMRE_HUMAN | Q8NCE2 | 429\| |
| LS14A_HUMAN | Q8ND56 | 375\| |
| DP13B_HUMAN | Q8NEU8 | 412\| |
| BD1L1_HUMAN | Q8NFC6 | 1742\|2386\| |
| NEUA_HUMAN | Q8NFW8 | 432\| |
| NEDD1_HUMAN | Q8NHV4 | 314\| |
| TM10A_HUMAN | Q8TBZ6 | 285\| |
| NEK9_HUMAN | Q8TD19 | 878\| |
| PKHO2_HUMAN | Q8TD55 | 295\| |
| MICA1_HUMAN | Q8TDZ2 | 837\| |
| IPO4_HUMAN | Q8TEX9 | 708\| |
| PRUN2_HUMAN | Q8WUY3 | 2536\|2598\|653\|1377\|357\| |
| LEO1_HUMAN | Q8WVC0 | 530\| |
| TPC12_HUMAN | Q8WVT3 | 160\| |
| HNRLL_HUMAN | Q8WVV9 | 505\| |
| NELFB_HUMAN | Q8WX92 | 115\| |
| PPM1E_HUMAN | Q8WY54 | 525\| |
| IRGQ_HUMAN | Q8WZA9 | 340\| |
| GBF1_HUMAN | Q92538 | 661\| |
| HS105_HUMAN | Q92598 | 376\|845\| |
| TBCD5_HUMAN | Q92609 | 676\| |
| ANS1A_HUMAN | Q92625 | 651\|849\| |
| AKAP1_HUMAN | Q92667 | 147\|165\|376\|438\| |
| ARC1A_HUMAN | Q92747 | 279\| |
| DDX17_HUMAN | Q92841 | 584\| |
| ARHG1_HUMAN | Q92888 | 911\| |
| FUBP2_HUMAN | Q92945 | 176\|436\| |
| ARHG2_HUMAN | Q92974 | 306\|478\|715\| |
| UBP7_HUMAN | Q93009 | 315\| |
| TSR2_HUMAN | Q969E8 | 114\| |
| NTAN1_HUMAN | Q96AB6 | 118\| |
| OTUL_HUMAN | Q96BN8 | 17\|47\| |
| EFHD2_HUMAN | Q96C19 | 53\| |
| FWCH2_HUMAN | Q96CP2 | 132\|64\| |
| F122A_HUMAN | Q96E09 | 193\| |
| SIR1_HUMAN | Q96EB6 | 574\| |
| RBM33_HUMAN | Q96EV2 | 726\| |
| DTBP1_HUMAN | Q96EV8 | 302\| |
| CYFP2_HUMAN | Q96F07 | 98\| |
| CCD97_HUMAN | Q96F63 | 78\| |
| MTNB_HUMAN | Q96GX9 | 16\| |
| ZC21A_HUMAN | Q96GY0 | 276\| |
| SAHH3_HUMAN | Q96HN2 | 353\| |
| MET2A_HUMAN | Q96IZ6 | 171\| |
| ZFP91_HUMAN | Q96JP5 | 182\|520\| |
| TOPK_HUMAN | Q96KB5 | 112\|70\|22\| |
| TRI47_HUMAN | Q96LD4 | 454\| |
| DOCK7_HUMAN | Q96N67 | 193\|1944\|2125\|1902\| |
| TRNT1_HUMAN | Q96Q11 | 373\| |
| XPO6_HUMAN | Q96QU8 | 299\| |
| UIMC1_HUMAN | Q96RL1 | 601\| |
| RANB9_HUMAN | Q96S59 | 513\|527\| |
| CK5P2_HUMAN | Q96SN8 | 249\| |
| CNN22_HUMAN | Q99439 | 164\|215\|175\| |
| PSMD1_HUMAN | Q99460 | 806\| |
| ANM1_HUMAN | Q99873 | 119\| |
| AKAP9_HUMAN | Q99996 | 1367\|3521\| |
| F118B_HUMAN | Q9BPY3 | 319\| |
| TRIR_HUMAN | Q9BQ61 | 165\| |
| MACD1_HUMAN | Q9BQ69 | 186\| |
| MBB1A_HUMAN | Q9BQG0 | 942\| |
| WAC_HUMAN | Q9BTA9 | 15\|553\| |
| DIDO1_HUMAN | Q9BTC0 | 498\|350\| |
| MCMBP_HUMAN | Q9BTE3 | 325\|200\| |
| KIFC1_HUMAN | Q9BW19 | 95\| |
| NADAP_HUMAN | Q9BWU0 | 730\| |
| NAA15_HUMAN | Q9BXJ9 | 238\|721\| |
| SRRT_HUMAN | Q9BXP5 | 412\| |
| FACD2_HUMAN | Q9BXW9 | 893\|310\| |
| ITPA_HUMAN | Q9BY32 | 146\| |
| TB182_HUMAN | Q9C0C2 | 1296\|1443\|794\|1114\|136\|1208\|1373\|1175\|749\| |
| UBE2O_HUMAN | Q9C0C9 | 375\|400\| |
| TANC1_HUMAN | Q9C0D5 | 1594\|1785\| |
| PITH1_HUMAN | Q9GZP4 | 14\| |
| EGLN1_HUMAN | Q9GZT9 | 127\| |
| ILKAP_HUMAN | Q9H0C8 | 301\| |
| DHX36_HUMAN | Q9H2U1 | 135\| |
| NELFA_HUMAN | Q9H3P2 | 471\| |
| SFR19_HUMAN | Q9H7N4 | 950\| |
| PHAX_HUMAN | Q9H814 | 51\| |
| CNO10_HUMAN | Q9H9A5 | 504\| |
| KT3K_HUMAN | Q9HA64 | 24\| |
| PKHA5_HUMAN | Q9HAU0 | 359\| |
| XPO5_HUMAN | Q9HAV4 | 1131\| |
| ARFG3_HUMAN | Q9NP61 | 241\| |
| ANLN_HUMAN | Q9NQW6 | 309\|469\|512\|353\|260\| |
| GEPH_HUMAN | Q9NQX3 | 284\|293\|419\| |
| DIAP3_HUMAN | Q9NSV4 | 1125\| |
| TXLNG_HUMAN | Q9NUQ3 | 101\| |
| EXOC1_HUMAN | Q9NV70 | 566\| |
| TBC13_HUMAN | Q9NVG8 | 387\|282\| |
| INT10_HUMAN | Q9NVR2 | 387\| |
| RPC5_HUMAN | Q9NVU0 | 456\| |
| NRK1_HUMAN | Q9NWW6 | 72\| |
| HPF1_HUMAN | Q9NWY4 | 29\| |
| CARF_HUMAN | Q9NXV6 | 178\| |
| TLS1_HUMAN | Q9NZ63 | 145\| |
| F120A_HUMAN | Q9NZB2 | 531\|919\| |
| SMAL1_HUMAN | Q9NZC9 | 108\| |
| CNOT2_HUMAN | Q9NZN8 | 175\| |
| OGFR_HUMAN | Q9NZT2 | 443\|417\|330\| |
| CHMP5_HUMAN | Q9NZZ3 | 20\| |
| VPS18_HUMAN | Q9P253 | 22\| |
| RCC2_HUMAN | Q9P258 | 428\| |
| ORC3_HUMAN | Q9UBD5 | 483\| |
| MALT1_HUMAN | Q9UDY8 | 71\| |
| CGBP1_HUMAN | Q9UFW8 | 92\| |
| LIMD1_HUMAN | Q9UGP4 | 148\| |
| CHRD1_HUMAN | Q9UHD1 | 86\| |
| EI2BD_HUMAN | Q9UI10 | 69\| |
| TR112_HUMAN | Q9UI30 | 33\| |
| GGA2_HUMAN | Q9UJY4 | 429\| |
| TF3C4_HUMAN | Q9UKN8 | 129\| |
| NUP50_HUMAN | Q9UKX7 | 151\| |
| PSME2_HUMAN | Q9UL46 | 91\| |
| DNPEP_HUMAN | Q9ULA0 | 144\|327\| |
| ASAP1_HUMAN | Q9ULH1 | 740\| |
| COR1C_HUMAN | Q9ULV4 | 420\|456\| |
| AKP8L_HUMAN | Q9ULX6 | 211\|128\| |
| POLI_HUMAN | Q9UNA4 | 560\| |
| CHIP_HUMAN | Q9UNE7 | 199\| |
| PP6R1_HUMAN | Q9UPN7 | 326\| |
| DICER_HUMAN | Q9UPY3 | 1641\|1621\| |
| MARE3_HUMAN | Q9UPY8 | 182\| |
| MTMR6_HUMAN | Q9Y217 | 569\| |
| LZTS1_HUMAN | Q9Y250 | 167\|385\| |
| VDAC3_HUMAN | Q9Y277 | 36\| |
| SAC2_HUMAN | Q9Y2H2 | 268\| |
| RRP44_HUMAN | Q9Y2L1 | 213\|799\| |
| TPPC8_HUMAN | Q9Y2L5 | 265\| |
| CRYL1_HUMAN | Q9Y2S2 | 125\| |
| AP3M1_HUMAN | Q9Y2T2 | 288\| |
| GIT1_HUMAN | Q9Y2X7 | 576\| |
| SNX24_HUMAN | Q9Y343 | 114\| |
| LC7L2_HUMAN | Q9Y383 | 348\| |
| PHOCN_HUMAN | Q9Y3A3 | 134\| |
| STRAP_HUMAN | Q9Y3F4 | 305\| |
| RBGP1_HUMAN | Q9Y3P9 | 476\| |
| WAC2C_HUMAN | Q9Y4E1 | 828\| |
| IRS2_HUMAN | Q9Y4H2 | 514\|1231\| |
| ATG4B_HUMAN | Q9Y4P1 | 189\| |
| PRC2C_HUMAN | Q9Y520 | 177\|953\| |
| PPME1_HUMAN | Q9Y570 | 238\| |
| TNPO3_HUMAN | Q9Y5L0 | 511\| |
| GMPPB_HUMAN | Q9Y5P6 | 245\| |
| TSSC4_HUMAN | Q9Y5U2 | 99\| |
| NS1BP_HUMAN | Q9Y6Y0 | 143\|274\| |
| S23IP_HUMAN | Q9Y6Y8 | 604\| |
| SHOT1_HUMAN | A0MZ66 | 550\| |
| LRC47_HUMAN | Q8N1G4 | 249\| |
| CASP2_HUMAN | P42575 | 343\| |
| LIN37_HUMAN | Q96GY3 | 28\| |
| CE022_HUMAN | Q49AR2 | 165\|195\| |
| STIP1_HUMAN | P31948 | 461\|26\| |
| RBBP7_HUMAN | Q16576 | 116\|97\| |
| SEP10_HUMAN | Q9P0V9 | 100\| |
| GEMI7_HUMAN | Q9H840 | 44\| |
| RECQ1_HUMAN | P46063 | 606\| |
| FTO_HUMAN | Q9C0B1 | 171\| |
| AGAP3_HUMAN | Q96P47 | 263\| |
| ERI1_HUMAN | Q8IV48 | 75\| |
| PI3R4_HUMAN | Q99570 | 899\| |
| RIPK1_HUMAN | Q13546 | 325\| |
| GCN1_HUMAN | Q92616 | 648\| |
| PRDX6_HUMAN | P30041 | 47\|91\| |
| SRP09_HUMAN | P49458 | 48\| |
| SYAC_HUMAN | P49588 | 671\|773\| |
| PLIN3_HUMAN | O60664 | 60\|39\| |
| LPP_HUMAN | Q93052 | 364\| |
| ZCCHL_HUMAN | Q96H79 | 121\| |
| PNPO_HUMAN | Q9NVS9 | 156\| |
| DUS3L_HUMAN | Q96G46 | 282\|54\| |
| DCTN4_HUMAN | Q9UJW0 | 258\| |
| TBC9B_HUMAN | Q66K14 | 444\| |
| CBX8_HUMAN | Q9HC52 | 261\| |
| SYTC_HUMAN | P26639 | 656\| |
| XPO1_HUMAN | O14980 | 119\| |
| KTNB1_HUMAN | Q9BVA0 | 344\| |
| ACLY_HUMAN | P53396 | 20\|845\| |
| IRAK4_HUMAN | Q9NWZ3 | 13\|128\| |
| TMF1_HUMAN | P82094 | 431\| |
| DIAP1_HUMAN | O60610 | 1227\| |
| NSUN2_HUMAN | Q08J23 | 502\|93\| |
| PTN23_HUMAN | Q9H3S7 | 628\| |
| ZCHC8_HUMAN | Q6NZY4 | 607\|393\| |
| L2GL1_HUMAN | Q15334 | 1059\| |
| PLST_HUMAN | P13797 | 104\|33\| |
| CKAP5_HUMAN | Q14008 | 1113\| |
| UBP8_HUMAN | P40818 | 809\| |
| ICAL_HUMAN | P20810 | 661\|381\|413\|328\|241\| |
| SRSF3_HUMAN | P84103 | 6\| |
| KNL1_HUMAN | Q8NG31 | 562\| |
| INT12_HUMAN | Q96CB8 | 379\| |
| PARN_HUMAN | O95453 | 169\|543\| |
| NTM1A_HUMAN | Q9BV86 | 195\| |
| TBC24_HUMAN | Q9ULP9 | 29\| |
| F263_HUMAN | Q16875 | 493\| |
| ZBT21_HUMAN | Q9ULJ3 | 152\|378\| |
| DYHC1_HUMAN | Q14204 | 4121\|1977\| |
| CND1_HUMAN | Q15021 | 286\|596\| |
| TCAL4_HUMAN | Q96EI5 | 34\| |
| CLAP2_HUMAN | O75122 | 976\| |
| ARFG1_HUMAN | Q8N6T3 | 96\| |
| OTUB1_HUMAN | Q96FW1 | 23\| |
| MSH6_HUMAN | P52701 | 88\| |
| CE170_HUMAN | Q5SW79 | 967\| |
| SEPT9_HUMAN | Q9UHD8 | 248\| |
| ELF2_HUMAN | Q15723 | 470\| |
| ACINU_HUMAN | Q9UKV3 | 546\| |
| AKA12_HUMAN | Q02952 | 470\|1314\|1139\| |
| SHIP2_HUMAN | O15357 | 926\| |
| CPPED_HUMAN | Q9BRF8 | 54\| |
| RANB3_HUMAN | Q9H6Z4 | 249\| |
| I2BP2_HUMAN | Q7Z5L9 | 65\| |
| DAXX_HUMAN | Q9UER7 | 427\|699\| |
| U520_HUMAN | O75643 | 238\| |
| ARPIN_HUMAN | Q7Z6K5 | 183\| |
| SYRC_HUMAN | P54136 | 32\| |
| AKAP2_HUMAN | Q9Y2D5 | 205\| |
| NR2C2_HUMAN | P49116 | 204\| |
| SRRM2_HUMAN | Q9UQ35 | 872\|1029\| |
| RN214_HUMAN | Q8ND24 | 149\| |
| PKHA4_HUMAN | Q9H4M7 | 438\| |
| PYGB_HUMAN | P11216 | 437\| |
| AEDO_HUMAN | Q96SZ5 | 239\| |
| ASPC1_HUMAN | Q9BZE9 | 48\|109\|224\| |
| STA5B_HUMAN | P51692 | 688\| |
| LANC2_HUMAN | Q9NS86 | 187\| |
| G3P_HUMAN | P04406 | 152\| |
| LRRF1_HUMAN | Q32MZ4 | 750\|644\| |
| SYMPK_HUMAN | Q92797 | 472\| |
| YAP1_HUMAN | P46937 | 343\| |
| ACSL3_HUMAN | O95573 | 649\| |
| CLU_HUMAN | O75153 | 1196\| |
| CASP8_HUMAN | Q14790 | 360\| |
| PSME3_HUMAN | P61289 | 92\| |
| TRI33_HUMAN | Q9UPN9 | 582\|786\| |
| PDCD4_HUMAN | Q53EL6 | 275\| |
| UBA3_HUMAN | Q8TBC4 | 28\| |
| UBR7_HUMAN | Q8N806 | 260\| |
| MAVS_HUMAN | Q7Z434 | 283\| |
| SOX10_HUMAN | P56693 | 190\| |
| VATG1_HUMAN | O75348 | 69\| |
| NIBA1_HUMAN | Q9BZQ8 | 891\| |
| SPF30_HUMAN | O75940 | 214\| |
| NF2IP_HUMAN | Q8NCF5 | 232\| |
| PDE12_HUMAN | Q6L8Q7 | 108\| |
| MACF1_HUMAN | Q9UPN3 | 5131\|7245\| |
| KTN1_HUMAN | Q86UP2 | 736\| |
| RB_HUMAN | P06400 | 853\| |
| RICTR_HUMAN | Q6R327 | 1576\| |
| CAND1_HUMAN | Q86VP6 | 413\| |
| RAN_HUMAN | P62826 | 112\| |
| PSMD2_HUMAN | Q13200 | 459\| |
| SOX6_HUMAN | P35712 | 594\| |
| LIPB2_HUMAN | Q8ND30 | 446\| |
| SYFB_HUMAN | Q9NSD9 | 195\| |
| BZW2_HUMAN | Q9Y6E2 | 270\| |
| COG7_HUMAN | P83436 | 505\| |
| EF1G_HUMAN | P26641 | 194\|266\|339\| |
| COPB_HUMAN | P53618 | 684\|623\| |
| 2A5D_HUMAN | Q14738 | 17\| |
| VAPA_HUMAN | Q9P0L0 | 60\| |
| EH1L1_HUMAN | Q8N3D4 | 346\| |
| PP6R3_HUMAN | Q5H9R7 | 844\|830\| |
| HNRH1_HUMAN | P31943 | 267\| |
| PR40A_HUMAN | O75400 | 39\| |
| PSMD9_HUMAN | O00233 | 59\| |
| WAC2A_HUMAN | Q641Q2 | 594\| |
| GTF2I_HUMAN | P78347 | 215\| |
| CHD4_HUMAN | Q14839 | 1594\|1827\| |
| HEM6_HUMAN | P36551 | 192\| |
| PP4R2_HUMAN | Q9NY27 | 22\| |
| NSL1_HUMAN | Q96IY1 | 224\| |
| KIF11_HUMAN | P52732 | 723\| |
| DCAF1_HUMAN | Q9Y4B6 | 784\| |
| HUWE1_HUMAN | Q7Z6Z7 | 2721\|3361\|3259\| |
| CD2AP_HUMAN | Q9Y5K6 | 540\| |
| MCM3_HUMAN | P25205 | 148\| |
| UBP24_HUMAN | Q9UPU5 | 1362\| |
| PK3CD_HUMAN | O00329 | 132\| |
| UACA_HUMAN | Q9BZF9 | 408\| |
| RS28_HUMAN | P62857 | 27\| |
| CSDE1_HUMAN | O75534 | 680\| |
| GPTC8_HUMAN | Q9UKJ3 | 508\| |
| VIME_HUMAN | P08670 | 328\| |
| TIGAR_HUMAN | Q9NQ88 | 114\|161\| |
| IMDH2_HUMAN | P12268 | 331\| |
| DNJA4_HUMAN | Q8WW22 | 368\| |
| I2BP1_HUMAN | Q8IU81 | 363\| |
| KAP2_HUMAN | P13861 | 101\| |
| DDX6_HUMAN | P26196 | 102\| |
| LIMA1_HUMAN | Q9UHB6 | 316\| |
| OGT1_HUMAN | O15294 | 323\| |
| NU160_HUMAN | Q12769 | 1166\| |
| TDRD7_HUMAN | Q8NHU6 | 333\| |
| INT9_HUMAN | Q9NV88 | 578\| |
| DCPS_HUMAN | Q96C86 | 37\| |
| VASP_HUMAN | P50552 | 334\| |
| HNRL2_HUMAN | Q1KMD3 | 308\| |
| FAD1_HUMAN | Q8NFF5 | 236\| |
| TBC15_HUMAN | Q8TC07 | 197\| |
| EFL1_HUMAN | Q7Z2Z2 | 953\| |
| ELP4_HUMAN | Q96EB1 | 218\| |
| DNMT1_HUMAN | P26358 | 1125\| |
| HDAC1_HUMAN | Q13547 | 408\| |
| CSK22_HUMAN | P19784 | 336\| |
| TCAL3_HUMAN | Q969E4 | 44\| |
| RO60_HUMAN | P10155 | 71\| |
| STRN4_HUMAN | Q9NRL3 | 337\| |
| STK24_HUMAN | Q9Y6E0 | 394\| |
| CLIC1_HUMAN | O00299 | 59\| |
| PABP1_HUMAN | P11940 | 339\|132\| |
| PABP4_HUMAN | Q13310 | 339\|132\| |
| HMGCL_HUMAN | P35914 | 323\| |
| UBP15_HUMAN | Q9Y4E8 | 264\| |
| IMPCT_HUMAN | Q9P2X3 | 226\| |
| MSH2_HUMAN | P43246 | 527\| |
| MCTS1_HUMAN | Q9ULC4 | 14\| |
| EEA1_HUMAN | Q15075 | 1134\| |
| PPIL4_HUMAN | Q8WUA2 | 426\| |
| MCAF1_HUMAN | Q6VMQ6 | 955\| |
| NCDN_HUMAN | Q9UBB6 | 98\| |
| SURF6_HUMAN | O75683 | 19\| |
| 1433T_HUMAN | P27348 | 134\| |
| RL10_HUMAN | P27635 | 105\| |
| NOP58_HUMAN | Q9Y2X3 | 439\|139\| |
| CPSF3_HUMAN | Q9UKF6 | 498\| |
| REN3B_HUMAN | Q9BZI7 | 319\| |
| JUPI2_HUMAN | Q9H910 | 118\| |
| PRKDC_HUMAN | P78527 | 2342\|25\| |
| ACTN4_HUMAN | O43707 | 499\| |
| IKKA_HUMAN | O15111 | 406\| |
| LRBA_HUMAN | P50851 | 1228\|950\| |
| UBR1_HUMAN | Q8IWV7 | 993\| |
| NOSIP_HUMAN | Q9Y314 | 8\| |
| UBE3A_HUMAN | Q05086 | 198\|108\| |
| STAT3_HUMAN | P40763 | 712\| |
| I2BPL_HUMAN | Q9H1B7 | 63\| |
| GNL3_HUMAN | Q9BVP2 | 113\| |
| CLMN_HUMAN | Q96JQ2 | 353\| |
| AAPK2_HUMAN | P54646 | 174\| |
| AAPK1_HUMAN | Q13131 | 185\| |
| COMT_HUMAN | P21964 | 223\| |
| NCK5L_HUMAN | Q9HCH0 | 783\| |
| PYRG1_HUMAN | P17812 | 491\| |
| MAAI_HUMAN | O43708 | 205\| |
| ATG7_HUMAN | O95352 | 524\| |
| DSN1_HUMAN | Q9H410 | 65\| |
| ESS2_HUMAN | Q96DF8 | 263\| |
| PURA_HUMAN | Q00577 | 272\| |
| RAI14_HUMAN | Q9P0K7 | 584\| |
| OSBL9_HUMAN | Q96SU4 | 720\| |
| DDX20_HUMAN | Q9UHI6 | 524\| |
| TRIP6_HUMAN | Q15654 | 47\| |
| CCD50_HUMAN | Q8IVM0 | 85\| |
| DPYL2_HUMAN | Q16555 | 504\| |
| RS16_HUMAN | P62249 | 25\| |
| NFKB2_HUMAN | Q00653 | 57\|432\| |
| SYCC_HUMAN | P49589 | 27\| |
| TOE1_HUMAN | Q96GM8 | 371\| |
| DIP2B_HUMAN | Q9P265 | 1019\| |
| POP1_HUMAN | Q99575 | 530\|358\| |
| FOXK1_HUMAN | P85037 | 404\| |
| R3HD4_HUMAN | Q96D70 | 29\| |
| NAA30_HUMAN | Q147X3 | 74\| |
| SPAG7_HUMAN | O75391 | 191\| |
| BORG4_HUMAN | Q9H3Q1 | 313\| |
| PIMT_HUMAN | P22061 | 102\| |
| LRCH3_HUMAN | Q96II8 | 531\| |
| CD11B_HUMAN | P21127 | 440\| |
| TUT7_HUMAN | Q5VYS8 | 911\| |
| GPX4_HUMAN | P36969 | 93\|134\| |
| MAGD2_HUMAN | Q9UNF1 | 516\| |
| KAD2_HUMAN | P54819 | 40\| |
| KLC4_HUMAN | Q9NSK0 | 113\| |
| TES_HUMAN | Q9UGI8 | 196\| |
| TADBP_HUMAN | Q13148 | 50\| |
| CUL4A_HUMAN | Q13619 | 633\| |
| CUL4B_HUMAN | Q13620 | 787\| |
| PLEC_HUMAN | Q15149 | 530\| |
| TBCK_HUMAN | Q8TEA7 | 386\| |
| NUCL_HUMAN | P19338 | 543\| |
| GABPA_HUMAN | Q06546 | 421\| |
| TMED8_HUMAN | Q6PL24 | 161\| |
| NUDC1_HUMAN | Q96RS6 | 376\| |
| CAN7_HUMAN | Q9Y6W3 | 197\| |
| TBA1A_HUMAN | Q71U36 | 347\| |
| TBA1C_HUMAN | Q9BQE3 | 347\| |
| 2ABA_HUMAN | P63151 | 334\| |
| 2ABB_HUMAN | Q00005 | 330\| |
| SIN3A_HUMAN | Q96ST3 | 1167\| |
| HNRPL_HUMAN | P14866 | 472\| |
| HS74L_HUMAN | O95757 | 540\| |
| ROCK2_HUMAN | O75116 | 649\| |
| APC7_HUMAN | Q9UJX3 | 131\| |
| ACACA_HUMAN | Q13085 | 1297\| |
| BCCIP_HUMAN | Q9P287 | 213\| |
| TPX2_HUMAN | Q9ULW0 | 536\| |
| PTBP1_HUMAN | P26599 | 23\| |
| CND2_HUMAN | Q15003 | 418\| |
| HERC4_HUMAN | Q5GLZ8 | 175\| |
| MRGBP_HUMAN | Q9NV56 | 170\| |
| CSTFT_HUMAN | Q9H0L4 | 222\| |
| RRAGC_HUMAN | Q9HB90 | 377\| |
| HPBP1_HUMAN | Q9NZL4 | 22\| |
| GCR_HUMAN | P04150 | 302\| |
| EI2BG_HUMAN | Q9NR50 | 281\| |
| CUL3_HUMAN | Q13618 | 636\| |
| TRM2A_HUMAN | Q8IZ69 | 463\| |
| MED15_HUMAN | Q96RN5 | 660\| |
| BRX1_HUMAN | Q8TDN6 | 52\| |
| UBR4_HUMAN | Q5T4S7 | 2554\| |
| KAZRN_HUMAN | Q674X7 | 312\| |
| DCAF8_HUMAN | Q5TAQ9 | 272\| |
| RUFY1_HUMAN | Q96T51 | 320\| |
| PRC2A_HUMAN | P48634 | 437\| |
| SCLY_HUMAN | Q96I15 | 22\| |
| SASH1_HUMAN | O94885 | 727\| |
| TPR_HUMAN | P12270 | 1149\| |
| CYBP_HUMAN | Q9HB71 | 173\| |
| DHX57_HUMAN | Q6P158 | 453\| |
| 1433E_HUMAN | P62258 | 97\| |
| DLGP5_HUMAN | Q15398 | 592\| |
| FUBP3_HUMAN | Q96I24 | 460\| |
| DAZP1_HUMAN | Q96EP5 | 85\| |
| ABRAL_HUMAN | Q9P1F3 | 39\| |
| LAD1_HUMAN | O00515 | 428\| |
| WIPI2_HUMAN | Q9Y4P8 | 393\| |
| SIN1_HUMAN | Q9BPZ7 | 149\| |
| PRDX3_HUMAN | P30048 | 229\| |
| ECHM_HUMAN | P30084 | 62\| |
| HS90B_HUMAN | P08238 | 564\| |
| FN3K_HUMAN | Q9H479 | 24\| |
| PPCE_HUMAN | P48147 | 57\| |
| TTC1_HUMAN | Q99614 | 8\| |
| KS6A3_HUMAN | P51812 | 229\| |
| KS6A1_HUMAN | Q15418 | 223\| |
| SH3G1_HUMAN | Q99961 | 277\| |
| PPR37_HUMAN | O75864 | 660\| |
| AR6P4_HUMAN | Q66PJ3 | 220\| |
| TLN1_HUMAN | Q9Y490 | 1353\| |
| SMRC1_HUMAN | Q92922 | 761\| |
| SNX8_HUMAN | Q9Y5X2 | 192\| |
| CBL_HUMAN | P22681 | 508\| |
| PDLI5_HUMAN | Q96HC4 | 213\| |
| GSK3B_HUMAN | P49841 | 14\| |
| DPH2_HUMAN | Q9BQC3 | 250\| |
| GSTO1_HUMAN | P78417 | 192\| |
| ZCCHV_HUMAN | Q7Z2W4 | 645\| |
| TACC1_HUMAN | O75410 | 219\| |
| DNM1L_HUMAN | O00429 | 505\| |
| IRF4_HUMAN | Q15306 | 194\| |
| SREK1_HUMAN | Q8WXA9 | 494\| |
| EIF3D_HUMAN | O15371 | 19\| |
| ABCF3_HUMAN | Q9NUQ8 | 102\| |
| LN28B_HUMAN | Q6ZN17 | 187\| |
| HPRT_HUMAN | P00492 | 106\| |
| YJU2_HUMAN | Q9BW85 | 275\| |
| RPP25_HUMAN | Q9BUL9 | 16\| |
| MITF_HUMAN | O75030 | 209\| |
| KIF1B_HUMAN | O60333 | 1635\| |

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
1. Cravatt et al. Activity-based protein profiling: from enzyme chemistry to proteomic chemistry. Annu Rev Biochem 2008, 77: 383-414.
2. Sadaghiani et al. Tagging and detection strategies for activity-based proteomics. Curr Opin Chem Biol 2007, 11(1): 20-28.
3. Niphakis & Cravatt. Enzyme inhibitor discovery by activity-based protein profiling. Annu Rev Biochem 2014, 83: 341-377.
4. Bachovchin & Cravatt. The pharmacological landscape and therapeutic potential of serine hydrolases. Nat Rev Drug Discov 2012, 11(1): 52-68.
5. Deu E, et al. New approaches for dissecting protease functions to improve probe development and drug discovery. Nat Struct Mol Biol 2012, 19(1): 9-16.
6. Patricelli et al. Functional interrogation of the kinome using nucleotide acyl phosphates. Biochemistry 2007, 46(2): 350-358.
7. Kumar et al. Activity-based probes for protein tyrosine phosphatases. Proc Natl Acad Sci USA 2004, 101(21): 7943-7948.
8. Vocadlo & Bertozzi. A strategy for functional proteomic analysis of glycosidase activity from cell lysates. Angew Chem Int Ed Engl 2004, 43(40): 5338-5342.
9. Liu et al. Activity-based protein profiling: the serine hydrolases. Proc Natl Acad Sci USA 1999, 96(26): 14694-14699.
10. Weerapana et al. Quantitative reactivity profiling predicts functional cysteines in proteomes. Nature 2010, 468(7325): 790-795.
11. Hacker et al. Global profiling of lysine reactivity and ligandability in the human proteome. Nat Chem 2017, 9(12): 1181-1190.
12. Lin et al. Redox-based reagents for chemoselective methionine bioconjugation. Science 2017, 355(6325): 597-602.
13. Matthews et al. Chemoproteomic profiling and discovery of protein electrophiles in human cells. Nat Chem 2017, 9(3): 234-243.
14. Parker et al. Ligand and Target Discovery by Fragment-Based Screening in Human Cells. Cell 2017, 168(3): 527-541 e529.
15. Hahm et al. Global targeting of functional tyrosines using sulfur-triazole exchange chemistry. Nat Chem Biol 2020, 16 (2), 150-159.
16. Brulet, et al. Liganding Functional Tyrosine Sites on Proteins Using Sulfur-Triazole Exchange Chemistry. J Am Chem Soc 2020, 142, 8270-8280.
17. Chen et al. Direct, Regioselective N-Alkylation of 1,3-Azoles, Org. Lett. 2016, 18 (1), 16-19.

It will be understood that various details of the presently disclosed subject matter can be changed without departing from the scope of the presently disclosed subject matter. Furthermore, the foregoing description is for the purpose of illustration only, and not for the purpose of limitation.

1. A method for identifying a reactive amino acid residue of a protein, the method comprising:
(a) providing a protein sample comprising isolated proteins, living cells, a cell lysate, or a biological organism;
(b) contacting the protein sample with a probe compound of Formula (I) for a period of time sufficient for the probe compound to react with at least one reactive amino acid in a protein in the protein sample, thereby forming at least one modified amino acid residue; and
(c) analyzing proteins in the protein sample or removed from the protein sample to identify at least one modified amino acid residue, thereby identifying at least one reactive amino acid residue of a protein; wherein the probe compound has a structure of Formula (I): 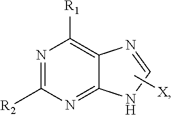
wherein:
X is a monovalent moiety comprising an alkyne moiety, a fluorophore moiety, a detectable labeling group, or a combination thereof; and
R₁ and R₂ are independently selected from the group consisting of H, halo, amino, alkyl, alkoxy, alkylthio, alkylamino, aryloxy, arylthiol, and arylamino, subject to the proviso that at least one of R₁ and R₂ is halo.

2. The method of claim 1, where the probe compound of Formula (I) has a structure of Formula (Ia): 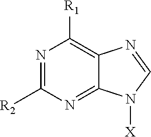
or a structure of Formula (Ib): 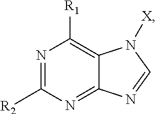
wherein:
X is a monovalent moiety comprising an alkyne moiety, a fluorophore moiety, a detectable labeling group, or a combination thereof; and
R₁ and R₂ are independently selected from the group consisting of H, halo, amino, alkyl, alkoxy, alkylthio, aryloxy, arylthiol, and arylamino, subject to the proviso that at least one of R₁ and R₂ is halo.

3. The method of claim 1, wherein the reactive amino acid residue is a cysteine residue.

4. The method of claim 1, wherein the modified amino acid residue has a structure of Formula (IIa-i): 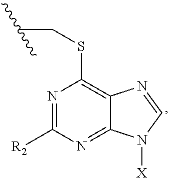
a structure of Formula (IIb-i): 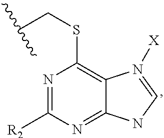
a structure of Formula (IIa-ii): 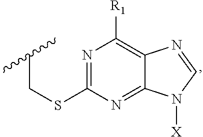
or a structure of Formula (IIb-ii): 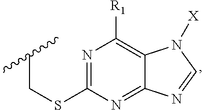
wherein:
X is a monovalent moiety comprising an alkyne moiety, a fluorophore moiety, a detectable labeling group, or a combination thereof;
R₁ is selected from the group consisting of H, halo, amino, alkyl, alkoxy, alkylthio, alkylamino, aryloxy, arylthio, and arylamino; and
R₂ is selected from the group consisting of H, halo, amino, alkyl, alkoxy, alkylthio, alkylamino, aryloxy, arylthio, and arylamino.

5. The method of claim 1, wherein R₁ and R₂ are selected from H, halo, and amino or wherein R₁ and R₂ are selected from H and halo.

6. The method of claim 1, wherein R₁ is chloro or fluoro.

7. The method of claim 1, wherein R₂ is chloro or fluoro.

8. The method of claim 1, wherein X is —CH₂—C≡CH.

9. The method of claim 1, wherein the probe compound is selected from the group consisting of 2,6-dichloro-7-(prop-2-yn-1-yl)-7H-purine, 2,6-dichloro-9-(prop-2-yn-1-yl)-9H-purine, 6-chloro-7-(prop-2-yn-1-yl)-7H-purine, 6-chloro-9-(prop-2-yn-1-yl)-9H-purine, 2-chloro-7-(prop-2-yn-1-yl)-7H-purine, 2-chloro-9-(prop-2-yn-1-yl)-9H-purine, 2,6-difluoro-7-(prop-2-yn-1-yl)-7H-purine, 2,6,-difluoro-9-(prop-2-yn-1-yl)-9H-purine, 6-chloro-2-fluoro-7-(prop-2-yn-1-yl)-7H-purine, 6-chloro-2-fluoro-9-(prop-2-yn-1-yl)-9H-purine, 6-chloro-2-amino-7-(prop-2-yn-1-yl)-7H-purine, and 6-chloro-2-amino-9-(prop-2-yn-1-yl)-9H-purine.

10. The method of claim 1, wherein the probe compound has a structure of Formula (Ib).

11. The method of claim 1, wherein the probe compound is 2,6-dichloro-7-(prop-2-yn-1-yl)-7H-purine.

12. The method of claim 1, wherein the analyzing of step (c) further comprises tagging the at least one modified reactive amino acid residue with a compound comprising a detectable labeling group, thereby forming at least one tagged reactive amino acid residue comprising said detectable labeling group.

13. The method of claim 12, wherein the detectable labeling group comprises biotin or a biotin derivative, optionally wherein the biotin derivative is desthiobiotin.

14. The method of claim 12, wherein the tagging comprises reacting an alkyne group in the X moiety of the at least one modified reactive amino acid residue with a compound comprising (i) an azide moiety and (ii) the detectable labeling group, optionally via a copper-catalyzed azide-alkyne cycloaddition (CuAAC) coupling reaction.

15. The method of claim 12, wherein the analyzing further comprises digesting proteins with trypsin to provide a digested protein sample comprising a protein fragment comprising the at least one tagged reactive amino acid moiety comprising the detectable labeling group.

16. The method of claim 15, wherein the analyzing further comprises enriching the digested protein sample for the detectable labeling group, optionally wherein the enriching comprises contacting the digested protein sample with a solid support comprising a binding partner of the detectable labeling group.

17. The method of claim 16, wherein the analyzing further comprises analyzing the enriched digested protein sample via liquid chromatography-mass spectrometry (LC-MS).

18. The method of claim 1, wherein the protein sample is a biological organism, optionally a mammal; wherein contacting the protein sample with the probe compound of Formula (I) comprises administering the probe compound of Formula (I) to the biological organism, optionally via oral administration or injection; and wherein prior to analyzing the proteins, tissues are removed from the biological organism and homogenized.

19. The method of claim 1, wherein:
providing the protein sample further comprises separating the protein sample into a first protein sample and a second protein sample;
contacting the protein sample with a probe compound of Formula (I) comprises contacting the first protein sample with a first probe compound of Formula (I) at a first probe concentration for a first period of time and contacting the second protein sample with one of the group consisting of: (b1) a second probe compound of Formula (I) at the first probe concentration for the first period of time, (b2) the first probe compound of Formula (I) at a second probe concentration for the first period of time, and (b3) the first probe compound of Formula (I) at the first probe concentration for a second period of time; thereby forming at least one modified reactive amino acid residue in said first and/or said second protein sample; and
analyzing proteins comprises analyzing the first and second protein samples to determine the presence and/or identity of a modified reactive amino acid residue in the first sample and the presence and/or identity of a modified reactive amino acid residue in the second sample.

20. The method of claim 1, wherein the protein sample comprises living cells and wherein providing the protein sample further comprises separating the protein sample into a first protein sample and a second protein sample and culturing the first protein sample in a first cell culture medium comprising heavy isotopes prior to the contacting of step (b), optionally wherein the first cell culture medium comprises ¹³C- and/or ¹⁵N-labeled amino acids, further optionally wherein the first cell culture medium comprises ¹³C-,¹⁵N-labeled lysine and arginine; and culturing the second protein sample in a second cell culture medium, wherein said second cell culture medium comprises a naturally occurring isotope distribution, prior to the contacting of step (b).

21. The method of claim 20, wherein one of the first and the second protein sample is cultured in the presence of an inhibitor of an enzyme known or suspected of being present in said first or second protein sample.

22. The method of claim 1, wherein the probe compound of Formula (I) comprises a detectable labeling group comprising a heavy isotope or wherein the analyzing of step (c) further comprises tagging the at least one modified amino acid residue with a compound comprising a detectable labeling group comprising a heavy isotope, optionally wherein the heavy isotope is carbon-13.

23. A probe compound for detecting a reactive amino acid residue, optionally a reactive cysteine residue, in a protein, wherein the probe compound is selected from the group consisting of 2,6-difluoro-7-(prop-2-yn-1-yl)-7H-purine, 2,6-difluoro-9-(prop-2-yn-1-yl)-9H-purine, and 6-chloro-2-fluoro-7-(prop-2-yn-1-yl)-7H-purine.

24. A compound having the structure of Formula (III): 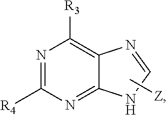
wherein:
Z is selected from the group consisting of cycloalkyl, acyl, substituted acyl,
—S(═O)₂—R₅,
—S(═O)₂—N(R₆)₂, —S(═O)₂—O—R₇, and 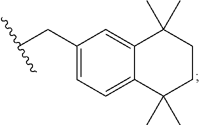 [SMILES: *Cc1ccc2c(c1)C(C)(C)CCC2(C)C]
R₃ and R₄ are independently selected from H, halo, alkyl, alkoxy, alkylamino, alkylthio, aryloxy, arylamino, and arylthiol, subject to the proviso that at least one of R₃ and R₄ is halo, optionally chloro or fluoro;
R₅ is heterocyclyl, substituted heterocyclyl, aryl or substituted aryl;
each R₆ is selected from H, alkyl, substituted alkyl, aralkyl, substituted aralkyl, aryl, and substituted aryl, or wherein the two R₆ together form an alkylene group; and
R₇ is selected from alkyl, substituted alkyl, aralkyl, substituted aralkyl, aryl and substituted aryl.

25. The compound of claim 24, wherein the compound of Formula (III) has a structure of Formula (IIIa): 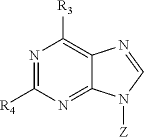
or a structure of Formula (IIIb): 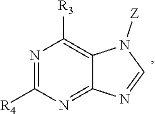
wherein:
Z is selected from the group consisting of cycloalkyl, acyl, substituted acyl,
—S(═O)₂—R₅,
—S(═O)₂—N(R₆)₂, —S(═O)₂—O—R₇, and 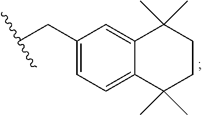 [SMILES: *Cc1ccc2c(c1)C(C)(C)CCC2(C)C]
R₃ and R₄ are independently selected from H, halo, alkyl, alkoxy, alkylamino, alkylthio, aryloxy, arylamino, and arylthiol, subject to the proviso that at least one of R₃ and R₄ is halo, optionally chloro or fluoro;
R₅ is heterocyclyl, substituted heterocyclyl, aryl or substituted aryl;
each R₆ is selected from H, alkyl, substituted alkyl, aralkyl, substituted aralkyl, aryl, and substituted aryl, or wherein the two R₆ together form an alkylene group; and
R₇ is selected from alkyl, substituted alkyl, aralkyl, substituted aralkyl, aryl and substituted aryl.

26. The compound of claim 24, wherein R₃ is selected from chloro, methyl, —SH—(CH₂)₃CH₃; —NH(CH₂)₃CH₃; and —O—(C₆H₄)CH₃.

27. The compound of claim 24, wherein R₄ is chloro or fluoro.

28. The compound of claim 24, wherein Z is acetyl, n-hexanoyl, n-dodecanoyl; cyclohexyl, —S(═O)₂—R₅, —S(═O)₂—N(R₆)₂, —S(═O)₂—O—R₇, and 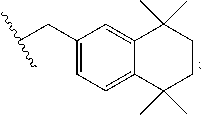
wherein R₅ is heterocyclyl or substituted phenyl; optionally wherein the substituted phenyl is alkoxy- or halo-substituted phenyl;
each R₆ is selected from alkyl and aralkyl, optionally methyl, ethyl or benzyl; and
R₇ is alkyl, optionally methyl.

29. The compound of claim 28, wherein Z is selected from

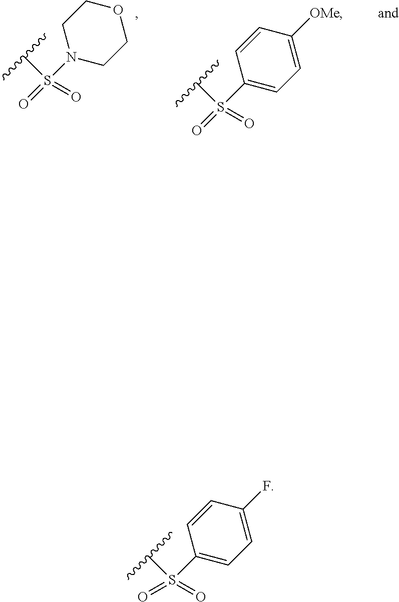 [SMILES: *S(=O)(=O)N1CCOCC1 ; *S(=O)(=O)c1ccc(OC)cc1 ; *S(=O)(=O)c1ccc(F)cc1]

30. The compound of claim 24, wherein the compound is selected from the group consisting of 4-((2,6-dichloro-7H-purin-7-yl)sulfonyl)morpholine, 4-((2,6-dichloro-9H-purin-9-yl)sulfonyl)morpholine, 2,6-dichloro-7-((4-fluorophenyl)sulfonyl)-7H-purine, 2,6-dichloro-9-((fluorophenyl)sulfonyl)-9H-purine, 2,6-dichloro-7-((4-methoxyphenyl)sulfonyl)-7H-purine, 2,6-dichloro-9-((4-methoxyphenyl)sulfonyl)-9H-purine, 2,6-dichloro-7-((5,5,8,8-tetramethyl-5,6,7,8-tetrahydronaphthalen-2-yl)methyl)-7H-purine, 2,6-dichloro-9-((5,5,8,8-tetramethyl-5,6,7,8-tetrahydronaphthalen-2-yl)methyl)-9H-purine, 1-(2,6-dichloro-9H-purin-9-yl)dodecan-1-one, 1-(2,6-dichloro-7H-purin-7-yl)hexan-1-one, 1-(2,6-dichloro-9H-purin-9-yl)hexan-1-one, 1-(6-chloro-2-fluoro-7H-purin-7-yl)hexan-1-one, 1-(6-chloro-2-fluoro-9H-purin-9-yl)hexan-1-one, 1-(2-chloro-6-methyl-9H-purin-9-yl)hexan-1-one, 2-chloro-9-cyclohexyl-6-methyl-9H-purine, 9-cyclohexyl-2-fluoro-6-methyl-9H-purine, 1-(6-(butylthio)-2-fluoro-9H-purin-9-yl)ethan-1-one, 1-(6-(butylthio)-2-chloro-9H-purin-9-yl)ethan-1-one, 1-(6-(butylthio)-2-chloro-7H-purin-7-yl)ethan-1-one, 1-(6-(butylthio)-2-fluoro-7H-purin-7-yl)ethan-1-one, 1-(2-chloro-6-(p-tolyloxy)-9H-purin-9-yl)ethan-1-one, 1-(2-fluoro-6-(p-tolyloxy)-9H-purin-9-yl)ethan-1-one, 1-(2-chloro-6-(p-tolyloxy)-7H-purin-7-yl)ethan-1-one, 1-(2-fluoro-6-(p-tolyloxy)-7H-purin-7-yl)ethan-1-one, 1-(6-(butylamino)-2-chloro-7H-purin-7-yl)ethan-1-one, 1-(6-(butylamino)-2-fluoro-7H-purin-7-yl)ethan-1-one, 1-(6-(butylamino)-2-chloro-9H-purin-9-yl)ethan-1-one, 1-(6-(butylamino)-2-fluoro-9H-purin-9-yl)ethan-1-one, 2,6-dichloro-N, N-diethyl-7H-purine-7-sulfonamide, 2,6-dichloro-N, N-diethyl-9H-purine-9-sulfonamide, N-benzyl-2,6-dichloro-N-methyl-9H-purine-9-sulfonamide, N-benzyl-2,6-dichloro-N-methyl-7H-purine-7H-sulfonamide, benzyl 2,6-dichloro-7H-purine-7-sulfonate, benzyl 2,6-dichloro-9H-purine-9-sulfonate, methyl 2,6-dichloro-9H-purine-9-sulfonate, and methyl 2,6-dichloro-7H-purine-7-sulfonate.

31. A compound, wherein said compound is 2,6-dichloro-7-(4-nitrobenzyl)-7H-purine.

32. A modified cysteine-containing protein comprising a modified cysteine residue wherein the modified cysteine residue is formed by the reaction of a cysteine residue with a non-naturally occurring purine-based compound wherein said non-naturally occurring purine-based compound is a compound having a structure of Formula (I): 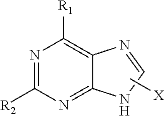
or
a compound having a structure of Formula (III′): 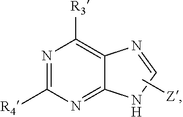
wherein:
X is a monovalent moiety comprising an alkyne moiety, a fluorophore moiety, a detectable labeling group, or a combination thereof;
Z′ is selected from the group consisting of alkyl, optionally —CH₂—CH═CH₂, substituted alkyl, cycloalkyl, heterocycloalkyl, acyl, substituted acyl, aralkyl, substituted aralkyl, —S(═O)₂—R₅′, —S(═O)₂—N(R₆)₂, and —S(═O)₂—O—R₇;
R₁ and R₂ are independently selected from the group consisting of H, halo, hydroxyl, thiol, amino, alkyl, alkoxy, alkylamino, alkylthio, aryloxy, arylamino, and arylthio, subject to the proviso that at least one of R₁ and R₂ is halo;
R₃′ and R₄′ are independently selected from H, halo, alkyl, alkylamino, alkylthio, alkoxy, aryloxy, arylamino, and arylthiol, subject to the proviso that at least one of R₃′ and R₄′ is halo;
R₅′ is heterocyclyl, substituted heterocyclyl, aryl or substituted aryl;
each R₆ is selected from H, alkyl, substituted alkyl, aralkyl, substituted aralkyl, aryl, and substituted aryl, or wherein the two R₆ together form an alkylene group; and
R₇ is selected from alkyl, substituted alkyl, aralkyl, substituted aralkyl, aryl and substituted aryl.

33. The modified cysteine-containing protein of claim 32, wherein said modified cysteine-containing protein comprises at least one modified cysteine residue comprising a structure of Formula (II-i): 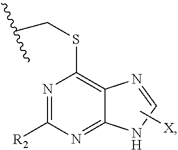
a structure of Formula (II-ii): 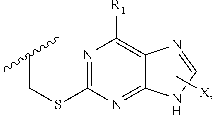
a structure of Formula (IV′-i): 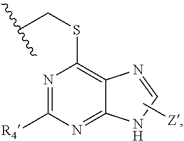
or a structure of Formula (IV′-ii): 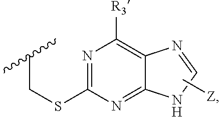
wherein:
X is a monovalent moiety comprising an alkyne moiety, a fluorophore moiety, a detectable labeling group, or a combination thereof;
Z′ is selected from the group consisting of alkyl, optionally —CH₂—CH═CH₂, substituted alkyl, cycloalkyl, heterocycloalkyl, acyl, substituted acyl, aralkyl, substituted aralkyl, —S(═O)₂—R₅′, —S(═O)₂—N(R₆)₂, and —S(═O)₂—O—R₇;
R₁ is selected from the group consisting of H, halo, hydroxyl, thiol, amino, alkyl, alkoxy, alkylamino, alkylthio, aryloxy, arylamino, and arylthio;
R₂ is selected from the group consisting of H, halo, hydroxyl, thiol, amino, alkyl, alkoxy, alkylamino, alkylthio, aryloxy, arylamino, and arylthio;
R₃′ is selected from the group consisting of H, halo, alkyl, alkylamino, alkylthio, alkoxy, aryloxy, arylamino, and arylthiol;
R₄′ is selected from the group consisting of H, halo, alkyl, alkylamino, alkylthio, alkoxy, aryloxy, arylamino, and arylthiol;
R₅′ is heterocyclyl, substituted heterocyclyl, aryl or substituted aryl;
each R₆ is selected from H, alkyl, substituted alkyl, aralkyl, substituted aralkyl, aryl, and substituted aryl, or wherein the two R₆ together form an alkylene group; and
R₇ is selected from alkyl, substituted alkyl, aralkyl, substituted aralkyl, aryl and substituted aryl.

34. The modified cysteine containing protein of claim 32, wherein the modified cysteine-containing protein is selenocysteine elongation factor (eEF-Sec) modified at cysteine 442, macrophage migration inhibitory factor modified at cysteine 81; or serine/threonine protein kinase 38-like modified at cysteine 235.

35. A method for modulating the activity of a protein comprising a reactive cysteine residue, wherein the method comprising contacting a protein comprising a reactive cysteine residue with a compound having a structure of Formula (III′): 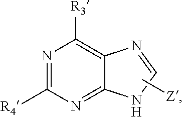
wherein:
Z′ is selected from the group consisting of alkyl, optionally —CH₂—CH═CH₂, substituted alkyl, cycloalkyl, heterocycloalkyl, acyl, substituted acyl, aralkyl, substituted aralkyl, —S(═O)₂—R₅′, —S(═O)₂—N(R₆)₂, and —S(═O)₂—O—R₇;
R₃′ and R₄′ are independently selected from H, halo, alkyl, alkylamino, alkylthio, alkoxy, aryloxy, arylamino, and arylthio, subject to the proviso that at least one of R₃′ and R₄′ is halo;
R₅′ is heterocyclyl, substituted heterocyclyl, aryl or substituted aryl;
each R₆ is selected from H, alkyl, substituted alkyl, aralkyl, substituted aralkyl, aryl, and substituted aryl, or wherein the two R₆ together form an alkylene group; and
R₇ is selected from alkyl, substituted alkyl, aralkyl, substituted aralkyl, aryl and substituted aryl.

36. The method of claim 35, wherein the compound having a structure of Formula (III′) is a compound having a structure of Formula (IIIa′): 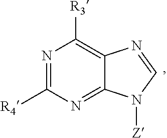
or a structure of Formula (IIIb′): 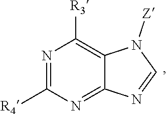
wherein Z′, R₃′, and R₄′ are as defined for Formula (III′).

37. The method of claim 35, wherein R₃′ is selected from chloro, fluoro, methyl, n-butylthio, n-butylamino, or —O—(C₆H₄)—OMe.

38. The method of claim 35, wherein Z′ is selected from —CH₂—CH═CH₂, C₂-C₁₂ acyl, cyclohexyl, benzyl, —CH₂—(C₆H₄)—NO₂, —S(═O)₂—R₅′, and 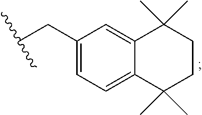
wherein R′₅ is selected from morpholinyl, 4-halophenyl, and 4-alkoxyphenyl.

39. The method of claim 35, wherein both R₃′ and R₄′ are chloro.

40. The method of claim 35, wherein the compound of Formula (III′) is selected from the group consisting of 4-((2,6-dichloro-7H-purin-7-yl)sulfonyl)morpholine, 4-((2,6-dichloro-9H-purin-9-yl)sulfonyl)morpholine, 2,6-dichloro-7-((4-fluorophenyl)sulfonyl)-7H-purine, 2,6-dichloro-9-((4-fluorophenyl)sulfonyl)-9H-purine, 2,6-dichloro-7-((4-methoxyphenyl)sulfonyl)-7H-purine, 2,6-dichloro-9-((4-methoxyphenyl)sulfonyl)-9H-purine, 2,6-dichloro-7-((5,5,8,8-tetramethyl-5,6,7,8-tetrahydronaphthalen-2-yl)methyl)-7H-purine, 2,6-dichloro-9-((5,5,8,8-tetramethyl-5,6,7,8-tetrahydronaphthalen-2-yl)methyl)-9H-purine, 2,6-dichloro-7-(4-nitrobenzyl)-7H-purine, 1-(2,6-dichloro-9H-purin-9-yl)dodecan-1-one, 1-(2,6-dichloro-7H-purin-7-yl)hexan-1-one, 1-(2,6-dichloro-9H-purin-9-yl)hexan-1-one, 1-(6-chloro-2-fluoro-7H-purin-7-yl)hexan-1-one, 1-(6-chloro-2-fluoro-9H-purin-9-yl)hexan-1-one, 1-(2-chloro-6-methyl-9H-purin-9-yl)hexan-1-one, 2-chloro-9-cyclohexyl-6-methyl-9H-purine, 9-cyclohexyl-2-fluoro-6-methyl-9H-purine, 1-(6-(butylthio)-2-fluoro-9H-purin-9-yl)ethan-1-one, 1-(6-(butylthio)-2-chloro-9H-purin-9-yl)ethan-1-one, 1-(6-(butylthio)-2-chloro-7H-purin-7-yl)ethan-1-one, 1-(6-(butylthio)-2-fluoro-7H-purin-7-yl)ethan-1-one, 1-(2-chloro-6-(p-tolyloxy)-9H-purin-9-yl)ethan-1-one, 1-(2-fluoro-6-(p-tolyloxy)-9H-purin-9-yl)ethan-1-one, 1-(2-chloro-6-(p-tolyloxy)-7H-purin-7-yl)ethan-1-one, 1-(2-fluoro-6-(p-tolyloxy)-7H-purin-7-yl)ethan-1-one, 1-(6-(butylamino)-2-chloro-7H-purin-7-yl)ethan-1-one, 1-(6-(butylamino)-2-fluoro-7H-purin-7-yl)ethan-1-one, 7-allyl-2,6-dichloro-7H-purine, 9-allyl-2,6-dichloro-9H-purine, 2,6-dichloro-7-benzyl-7H-purine, 2,6-dichloro-9-benzyl-9H-purine, 2,6-dichloro-7-(4-nitrobenzyl)-7H-purine, 2,6-dichloro-9-(4-nitrobenzyl-9H-purine, 2-(2,6-dichloro-9H-purin-9-yl)-5-(hydroxymethyl)tetrahydrofuran-3,4-diol, 1-(6-(butylamino)-2-chloro-9H-purin-9-yl)ethan-1-one, 1-(6-(butylamino)-2-fluoro-9H-purin-9-yl)ethan-1-one, 2,6-dichloro-N,N-diethyl-7H-purine-7-sulfonamide, 2,6-dichloro-N, N-diethyl-9H-purine-9-sulfonamide, N-benzyl-2,6-dichloro-N-methyl-9H-purine-9-sulfonamide, N-benzyl-2,6-dichloro-N-methyl-7H-purine-7H-sulfonamide, benzyl 2,6-dichloro-7H-purine-7-sulfonate, benzyl 2,6-dichloro-9H-purine-9-sulfonate, methyl 2,6-dichloro-9H-purine-9-sulfonate, and methyl 2,6-dichloro-7H-purine-7-sulfonate.

41. The method of claim 35, wherein modulating an activity of a protein comprising a reactive cysteine residue comprises inhibiting an activity of the protein comprising a reactive cysteine residue.

42. The method of claim 35, wherein modulating an activity of a protein comprising a reactive cysteine residue comprises activating an activity of the protein comprising a reactive cysteine residue.

43. The method of claim 35, wherein modulating an activity of a protein comprising a reactive cysteine residue comprises blocking a protein-protein interaction of the protein comprising a reactive cysteine residue.

44. The method of claim 35, wherein modulating an activity of a protein comprising a reactive cysteine residue comprises disrupting a protein-RNA interaction of the protein comprising a reactive cysteine residue.

45. The method of claim 35, wherein modulating an activity of a protein comprising a reactive cysteine residue comprises disrupting a protein-DNA interaction of the protein comprising a reactive cysteine residue.

46. The method of claim 35, wherein modulating an activity of a protein comprising a reactive cysteine residue comprises disrupting a protein-lipid interaction of the protein comprising a reactive cysteine residue.

47. The method of claim 35, wherein modulating an activity of a protein comprising a reactive cysteine residue comprises disrupting a protein-metabolite interaction of the protein comprising a reactive cysteine residue.

48. The method of claim 35, wherein modulating an activity of a protein comprising a reactive cysteine residue comprises disrupting subcellular localization of the protein comprising a reactive cysteine residue.

49. The method of claim 35, wherein modulating an activity of a protein comprising a reactive cysteine residue comprises triggering recruitment of an E3 ligase for targeted degradation of the protein comprising a reactive cysteine residue.
